(19) United States
(12) Patent Application Publication
(10) Pub. No.: US 2019/0201500 A1
(43) Pub. Date: Jul. 4, 2019

(54) COMPOSITIONS AND METHODS OF FAS INHIBITION

(71) Applicants: ONL THERAPEUTICS, INC., Ann Arbor, MI (US); SCHEPENS EYE RESEARCH INSTITUTE INC., MASSACHUSETTS EYE AND EAR INFIRMARY, Boston, MA (US)

(72) Inventors: David Zacks, Ann Arbor, MI (US); Andrew Kocab, Farmington Hills, MI (US); John Freshley, Ann Arbor, MI (US); Meredith Gregory-Ksander, Boston, MA (US); Anitha Krishnan, Braintree, MA (US); Jana van de Goor, Ann Arbor, MI (US); Alexander J. Bridges, Saline, MI (US)

(21) Appl. No.: 16/359,479

(22) Filed: Mar. 20, 2019

Related U.S. Application Data
(63) Continuation-in-part of application No. 15/570,948, filed on Oct. 31, 2017.
(60) Provisional application No. 62/700,097, filed on Jul. 18, 2018.
(60) Provisional application No. 62/645,769, filed on Mar. 20, 2018.
(60) Provisional application No. 62/155,711, filed on May 1, 2015.

(51) Int. Cl. A61K 38/45; A61K 9/00; A61K 48/00

(52) U.S. Cl. CPC A61K 38/45; A61K 48/005; A61K 9/0019

(57) ABSTRACT

Provided herein are compositions including peptides, pharmaceutical preparations thereof, and methods of preventing photoreceptor death therewith and protecting of retinal cells, including, but not limited to, photoreceptors and retinal pigment epithelium, from Fas- or TRAIL-mediated apoptosis.

Also, described are compositions and methods for preventing, treating or ameliorating an inflammation-mediated and/or complement-mediated disease or condition in a subject comprising administering to the subject a Fas inhibitor, its derivative, a pharmaceutically acceptable salt thereof, of a gene therapy encoding the Fas inhibitor in an amount effective to inhibit Fas signaling.

### RELATED APPLICATIONS

The present patent document is a continuation-in-part application claiming priority to U.S. patent application Ser. No. 15/570,948, filed Oct. 31, 2017, which is a § 371 filing based on PCT Application Serial No. PCT/US2016/030098, filed Apr. 29, 2016, which claims the benefit of the filing date under 35 U.S.C. § 119(e) of Provisional U.S. Patent Application Ser. No. 62/155,711, filed May 1, 2015, which are hereby incorporated by reference.

The present patent document also claims the benefit of the filing date under 35 U.S.C. § 119(e) of Provisional U.S. Patent Application Ser. Nos. 62/645,769, filed Mar. 20, 2018 and 62/700,097, filed Jul. 18, 2018, which are hereby incorporated by reference.

All patents, patent applications and publications, and other literature references cited herein are hereby incorporated by reference in their entirety. The disclosures of these publications in their entireties are hereby incorporated by reference into this application in order to more fully describe the state of the art as known to those skilled therein as of the date of the invention described and claimed herein.

### FEDERALLY SPONSORED RESEARCH OR DEVELOPMENT

This invention was made with Government support under Grant No. R44EY022512, awarded by the National Institute of Health (NIH). The Government has certain rights in this invention.

### BACKGROUND

Peptide compositions that are protective of cells, especially retinal cells, including, but not limited to, photoreceptors, retinal pigment epithelium (RPE), and retinal ganglion cells, which receive visual information from photoreceptors, from extrinsic pathway-mediated cell death, such as Fas-mediated apoptosis, TRAIL-mediated apoptosis, TNF-mediated necroptosis, and pyroptosis, and methods of using the compositions are described.

Several major causes of vision loss, such as retinal detachment, glaucoma and macular degeneration, have a significant component of apoptotic signaling, which in turn leads to programmed cell death in certain very important types of cells in the retina. Three of these cell types are the retinal pigmented epithelial cells, where loss is seen in retinal bleaching, retinitis pigmentosa and the dry form of age-related macular degeneration, the retinal ganglionic cells, where loss is seen in glaucoma, and the photoreceptor cells themselves, the primary visual signaling cells and whose loss is the ultimate cause of vision loss from retinal diseases.

Retinal detachment (RD), defined as the separation of the neurosensory retina from subjacent RPE, results in the apoptotic death of photoreceptor cells (Cook et al. 1995; 36(6):990-996; Hisatomi et al. Curr Eye Res. 2002; 24(3):161-172; Zacks et al. Invest Ophthalmol Vis Sci. 2003; 44(3):1262-1267. Yang et al. Invest Ophthalmol Vis Sci. 2004; 45(2):648-654; herein incorporated by reference in their entireties). Rodent and feline models of RD have demonstrated the activation of pro-apoptotic pathways nearly immediately after the retina becomes separated from the RPE (Cook et al. 1995; 36(6):990-996; Hisatomi et al. Curr Eye Res. 2002; 24(3):161-172; Zacks et al. Invest Ophthalmol Vis Sci. 2003; 44(3):1262-1267. Yang et al. Invest Ophthalmol Vis Sci. 2004; 45(2):648-654; herein incorporated by reference in their entireties). Histological markers of apoptosis such as terminal deoxynucleotidyl transferase nick end label (TUNEL) staining reach a peak at approximately three days after RD, with apoptotic activity and progressive cell death persisting for the duration of the detachment period. This has also been validated in human retinal detachments (Arroyo et al. Am J Ophthalmol. 2005 April; 139(4):605-10). Clinical experience in the repair of retinal detachments, however, has demonstrated that there is a window of opportunity for repair with preservation of some visual acuity, but that the visual acuity drops significantly as the time between detachment and repair extends (Burton. Trans Am Ophthalmol Soc. 1982; 80:475-497; Ross et al. Ophthalmology. 1998; 105(11):2149-2153; Hassan et al. Ophthalmology. 2002; 109(1):146-152; herein incorporated by reference in their entireties). The rapid rate of activation of pro-apoptosis pathways and the slower rate of visual loss suggests that intrinsic neuroprotective factors may become activated within the neural retina, and may serve to counter-balance the effects of the pro-apoptotic pathways activated by retinal-RPE separation.

Age-Related Macular Degeneration (AMD) is the leading cause of permanent vision loss in the United States (Bourne et al. Br J Ophthalmol. 2014; 98:629-638; Klein et al. Arch Ophthalmol. 2011; 129:75-80; Cruciani et al. Clin Ter. 2011; 162:e35-42). Death of the outer retina (defined here as the complex of retinal pigment epithelium (RPE) and photoreceptor (PR) cells) is the root cause of vision loss in AMD and limits the effectiveness of current treatments (Murakami et al. Prog Retin Eye Res. 2013; 37:114-140; Huckfeldt and Vavvas. Int Ophthalmol Clin. 2013; 53:105-117). Disruption of PR-RPE homeostasis results in PR death. Fas was significantly expressed in eyes of people with advanced AMD, defined as wet or atrophic, compared to healthy controls and was most concentrated around active neovascular and atrophic lesions (Dunaief et al. Arch Ophthalmol. 2002; 120:1435-1442). RPE is sensitive to Fas-mediated apoptosis under stress conditions that occur during AMD progression, such as inflammation or oxidative stress, and higher concentrations of soluble Fas ligand were identified in AMD patients when compared to their age-matched healthy counterparts (Jiang et al. Invest Ophthalmol Vis Sci. 2008; 37:114-140). Similarly, oxidative stress, which occurs during AMD progression, results in the increased expression of Fas in the RPE (Lin et al. Invest Ophthalmol Vis Sci. 2011; 52:6308-6314) and the death of the RPE that occurs in conditions of oxidative stress is dependent on Fas signaling (Wang et al. Apoptosis. 2012; 17:1144-1155). Additionally, Fas has been directly linked to RPE cell death induced by Alu RNA accumulation, another recognized factor of AMD pathology (Kim et al. Proc Natl Acad Sci USA. 2014; 111:16082-16087). The TRAIL-R1 receptor (DR4), which operates partially through the same pathway has been shown to be a genetic risk factor for The TRAIL-R1 receptor (DR4), which operates partially through the same pathway has been shown to be a genetic risk factor for Age-related macular degeneration. (Miyake et al. Invest Ophthalmol Vis Sci 56, 5353 (2015).

Fas has also been implicated in glaucoma-associated retinal ganglion cell death (Gregory et al. PLoS One. 2011; 6(3):e17659). Furthermore, intraocular pressure (IOP) is a major risk factor for glaucoma progression, and animal models of IOP exhibit increased Fas and FasL expression (Ju et al. Brain Res. 2006; 1122(1): 209-221) and retinal ganglion cell death by apoptosis (Ji et al. Vision Res. 2005; 45(2): 169-179). While control of IOP is a main tenet of clinical treatment of glaucoma, there are a substantial number of patients that continue to experience disease progression even after proper control of IOP, and additional work has reinforced the notion that additional contributing factors to glaucoma may need to be addressed (Kamat et al. Semin Ophthalmol. 2016; 31(1-2):147-154).

Apoptosis (programmed cell death) plays a central role in the development and homeostasis of all multi-cellular organisms. Alterations in apoptotic pathways have been implicated in many types of human pathologies, including developmental disorders, cancer, autoimmune diseases, as well as neuro-degenerative disorders, and retinal degradation. It is a tightly regulated pathway governing the death processes of individual cells and can be initiated either extrinsically or intrinsically. The latter is an intracellular mechanism triggered by the mitochondria while the former involves the interaction of a ‘death receptor’ with its corresponding ligand at the cell membrane. Thus, the programmed cell death pathways have become attractive targets for development of therapeutic agents. In particular, since it is conceptually easier to kill cells than to sustain cells, attention has been focused on anti-cancer therapies using pro-apoptotic agents. However, there are many diseases where inappropriate activation of apoptotic pathways leads to the degeneration of tissues, and treatments have to be devised to block whichever apoptotic pathway, intrinsic or extrinsic, has been activated in this particular disease pathology.

The Fas receptor is the most common of the death receptors involved in apoptosis in degenerative diseases of the retina. (Chinsky et al. Curr Opin Ophthalmol. 2014 25(3); 228-233) Fas is a typical cytokine cell surface receptor, and is activated by trimerization when it binds to its trimeric cognate ligand FasL. Stressed retinal cells, for example photoreceptors after RD, upregulate the Fas receptor. Invading immune cells, attracted by the stress response, express the transmembrane protein Fas ligand (FasL) on their surface. FasL binds with the Fas receptors on the retinal cells, leading to a rapid activation of the extrinsic cell death pathway with signaling through the caspase cascade. Initially, the “initiator” caspase-8 is cleaved to an active form, which in turn activates caspase 3, a downstream “executioner” of the apoptotic cell death pathway. However, in the eyes of mice infected with murine cytomegalovirus, Fas, as well as the related death receptors TNFR1 and TRAIL, have been shown to be activated, and this activity can lead to apoptosis, necroptosis, and pyroptosis in cells of the eye. (Chien and Dix J Virol 86, 10961 (2012))

It has been shown that photoreceptor cells in culture are very sensitive to apoptosis induced by FasL suggesting that FasL-induced apoptosis is a major contributor to vision loss in retinal diseases. (Burton. Trans Am Ophthalmol Soc. 1982; 80:475-497; Ross et al. Ophthalmology. 1998; 105(11):2149-2153; Hassan et al. Ophthalmology. 2002; 109(1):146-152.) Furthermore, a small peptide inhibitor of the Fas receptor, Met-12, H⁶⁰HIYLGAVNYIY⁷¹ (SEQ ID NO:2) derived from the Fas-binding extracellular domain of the oncoprotein Met, (Zou et al. Nature Medicine 13, 1078 (2007) has been shown to be photoreceptor protective, both in cell culture experiments, and in the setting of separation of the retinal and retinal pigment epithelium and other ocular conditions or diseases. (Besirli et al., Invest Ophthalmol Vis Sci., 51(4):2177-84 (2010); U.S. Pat. No. 8,343,931; herein incorporated by reference in their entireties). Furthermore c-Met, presumably using the same binding domain with homology to Met-12, FasL, TNAα and TRAIL has been shown to block TRAIL-induced apoptosis in various tumors. (Du et al. PLoS One 9, e95490 (2014))

The Met-12 peptide itself has biopharmaceutical properties, dominated by its extremely poor aqueous solubility. Experiments have clearly shown that Met-12 has to be dosed as a solution, both in vitro and in vivo, to show optimal activity, and producing such solutions in a largely aqueous medium has proven to be very difficult, especially under conditions which are acceptable for intravitreal injection. Dosing of suspensions or gels of Met-12 leads to major losses of potency. For example, even an apparently clear 10 mg/mL solution of Met-12 in 20 mM citrate buffer pH 2.8 showed a considerable loss of material upon filtration, and when used in both the in vitro and in vivo assays described below, led to at least a fivefold loss in activity. Despite extensive development work, the only solution formulations of Met-12 which have been found involve some very low pH solution injections (≤pH 2.8) or neat DMSO injections, all of which are suboptimal for intravitreal injections.

As such, peptide compositions that are protective of retinal cells, including, but not limited to, photoreceptors, retinal ganglionic cells and retinal pigment epithelium, from extrinsic pathway cell death, including Fas- and TRAIL-mediated apoptosis, that are easy to formulate in a solution or suspension, which can be delivered into the eye in a way to create sufficient exposure, without the use of excipients which may cause ocular (or other) toxicity, and that are easy to use, are still needed to help preserve vision.

Fas (CD95/APO-1) and its specific ligand (FASL/CD95L) are members of the tumor necrosis factor (TNF) receptor (TNF-R) and TNF families of proteins, respectively.

Interaction between Fas and FASL triggers a cascade of subcellular events that results in a definable cell death process in Fas-expressing targets. Fas is a 45 kDa type I membrane protein expressed constitutively in various tissues, including spleen, lymph nodes, liver, lung, kidney and ovary. (Leithauser, F. et al, Lab Invest, 69:415-429 (1993); Watanabe-Fukunaga, R. et al, J Immunol, 148:1274-1279 (1992)). FASL is a 40 kDa type II membrane protein, and its expression is predominantly restricted to lymphoid organs and perhaps certain immune-privileged tissues. (Suda, T. et al, Cell, 75:1169-1178 (1993); Suda, T. et al, J Immunol, 154:3806-3813 (1995)). In humans, FASL can induce cytolysis of FAS-expressing cells, either as a membrane-bound form or as a 17 kDa soluble form, which is released through metalloproteinase-mediated proteolytic shedding. (Kayagaki, N. et al, J Exp Med, 182:1777-1783 (1995); Mariani, S. M. et al, Eur J Immunol, 25:2303-2307 (1995)).

Binding of Fas ligands (FasL) to Fas receptor can elicit apoptotic signals either via classical pathways or via indirect pathways (Mundle & Raza., Trends. Immuno., 23:187-194 (2002)). Independently, Fas and FasL stimulation alone can induce cell proliferation (Aggarwal et al., FEBS Lett, 364:5-8 (1995); Freiberg et al, J Invest Dermatol, 108:215-219 (1997); Jelaska & Korn, J. Cell. Physiol, 175:19-29 (1998); Suzuki et al, J Immunol, 165:5537-5543 (2000); Suzuki et al, J. Exp. Med., 187: 123-8 (1998)). Membrane bound TNF superfamily members including FasL has been show to “reverse-signal” via their membrane attach cytoplasmic tail and thus they also possess a “bi-directional” signaling (Sun & Fink, J. Immuno., 179:4307-4312 (2007)). These studies suggest that small molecules, such as Kp 7 and mimetics thereof, which bind to both Fas and FasL can regulate Fas receptor signaling in a tissue-specific manner can be used to treat a variety of autoimmune pathologies.

The FASL/FAS system has been implicated in the control of the immune response and inflammation, the response to infection, neoplasia, and death of parenchymal cells in several organs. (Nagata et al supra; Biancone, L. et al., J Exp Med, 186:147-152 (1997); Krammer, P. H. Adv Immunol, 71:163-210 (1999); Seino, K. et al, J Immunol, 161:4484-4488 (1998)). Defects of the FASL/FAS system can limit lymphocyte apoptosis and lead to lymphoproliferation and autoimmunity. A role for FASL-FAS in the pathogenesis of rheumatoid arthritis, Sjogren's syndrome, multiple sclerosis, viral hepatitis, renal injury, inflammation, aging, graft rejection, HIV infection and a host of other diseases has been proposed. (Famularo, G., et al., Med. Hypotheses, 53:50-62 (1999)). FAS mediated apoptosis is an important component of tissue specific organ damage, such as liver injury that has been shown to be induced through the engagement of the FAS-FASL receptor system. (Kakinuma, C. et al., Toxicol Pathol, 27: 412-420 (1999); Famularo, G., et al., Med Hypotheses, 53: 50-62 (1999); Martinez, O. M. et al., Int Rev Immunol, 18:527-546 (1999); Kataoka, Y. et al, Immunology, 103:310-318 (2001); Chung, C S. et al, Surgery, 130:339-345 (2001); Doughty, L. et al, Pediatr Res, 52:922-927 (2002)).

Glaucoma is an eye disorder characterized by increased pressure inside the eye (“intraocular pressure” or “IOP”), excavation of the optic nerve head and gradual loss of the visual field. An abnormally high IOP is commonly known to be detrimental to the eye, and there are clear indications that, in glaucoma patients, this probably is the most important factor causing degenerative changes in the retina. The pathophysiological mechanism of open angle glaucoma is, however, still unknown. Unless treated successfully glaucoma will lead to blindness sooner or later, its course towards that stage is typically slow with progressive loss of the vision. IOP is the fluid pressure inside the eye. Tonometry is the method eye care professionals use to determine this. IOP is an important aspect in the evaluation of patients at risk of glaucoma. Most tonometers are calibrated to measure pressure in millimeters of mercury (mmHg).

In retinal cells, Fas receptor is activated by Fas ligand (FasL). Fas mediates cell death directly via multiple pathways: extrinsic apoptosis (through caspase cascade), intrinsic apoptosis (through Bid/Bax), and necroptosis (through RIPK1/3). Fas also mediates cell death indirectly through multiple immune response pathways: inflammasome (NLRP3, IL1β, TNFα), inflammasome-independent IL1β activation, HMGB1 nuclear release and secretion, and others yet to be determined.

Consequently, the FASL-FAS pathway represents an important general target for therapeutic intervention.

As such, there still exists a need for developing Fas inhibitors, compositions including Fas inhibitors, and methods of using the Fas inhibitors in order to prevent or ameliorate various diseases or conditions.

### SUMMARY

One embodiment relates to a method for preventing, treating or ameliorating an inflammation-mediated and/or complement-mediated disease or condition in a subject comprising administering to the subject a Fas inhibitor, its derivative, a pharmaceutically acceptable salt thereof, or a gene therapy encoding the Fas inhibitor in an amount effective to inhibit Fas signaling, wherein the inhibition of Fas signaling results in at least one (or at least two, or at least three, or at least four, etc., or all) of the following: reduction of expression or concentration of at least one Fas-mediated inflammation-related gene or protein (e.g. TNFα, IL-1β, IP-10, IL-18, MIP1α, IL-6, GFAP, MIP2, MCP-1, or MIP-1β); reduction of expression or concentration of at least one Fas-mediated complement-related gene or protein (e.g., complement component 3 (C3) and complement component 1q (C1q)); reduction of gene or protein expression or concentration of Caspase 8; reduction of gene or protein expression or concentration of one or more components of the inflammasome (e.g., NLRP3 and NLRP2); reduction of gene or protein expression or concentration of one or more C-X-C motif chemokines (e.g., CXCL2 (MIP-2α) and CXCL10 (IP-10)); reduction of gene or protein expression or concentration of one or more C-X3-C motif chemokines (e.g., CX3CL1 (fractalkine)); reduction of gene or protein expression or concentration of one or more C-C motif chemokines (e.g., CCL2 (MCP-1), CCL3 (MIP-1α), and CCL4 (MIP-1β)); reduction of gene or protein expression or concentration of toll-like receptor 4 (TLR4); reduction of gene or protein expression or concentration of one or more interleukin cytokines (e.g., IL-1β, IL-18, and IL-6); reduction of gene or protein expression or concentration of one or more TNF superfamily cytokines (e.g., TNFα); reduction of Fas-mediated Müller cell activation as indicated by reduced GFAP gene or protein expression or concentration; or increase of expression or concentration or prevent the reduction of expression or concentration of at least one pro-survival gene or protein, thereby preventing, treating, or ameliorating the disease or condition in the subject. The Fas inhibitor may be selected from the group consisting of: Met protein, derivatives, fragments, pharmaceutically acceptable salts thereof; Met-12, derivatives, fragments, pharmaceutically acceptable salts thereof; SEQ ID NOs: 1-8, derivatives, fragments, pharmaceutically acceptable salts thereof; or a gene therapy agents encoding the Fas inhibitor. The subject may have or is at risk of having the inflammation-mediated and/or complement-mediated disease or condition. The inflammation-mediated and/or complement-mediated disease or condition may be retinal disease (e.g., glaucoma, retinal detachment, AMD (dry and wet), diabetic retinopathy, Uveitis, retinal vein occlusion, inherited retinal degenerations, including retinitis pigmentosa, or NAION), immunological disease, cancer, amyloid disease (e.g., Alzheimer's disease, type-2 diabetes, Huntington's disease, ALS, or Parkinson's disease), an injury caused by ischemia or reperfusion (e.g., stroke), autoimmune disease (e.g., allergy, lupus, or rheumatoid arthritis), neurodegeneration, and diseases of the central nervous system (e.g., neuropathy or a demyelinating disease selected from the group consisting of multiple sclerosis and inflammatory demyelinating diseases). The Fas inhibitor, its derivative, fragment, the gene therapy product, its corresponding interfering RNA (RNAi), or the pharmaceutically acceptable salt thereof may be administered in a pharmaceutical composition comprising the Fas inhibitor, its derivative, fragment, pharmaceutically acceptable salt, or a gene therapy that encodes the Fas inhibitor; and a pharmaceutically acceptable additive, such as carriers, excipients, disintegrators or disintegrating aids, binders, lubricants, coating agents, pigments, diluents, bases, dissolving agents or solubilizers, isotonic agents, pH regulators, stabilizers, propellants, and adhesives. In the method, the Fas inhibitor, its derivative, or the pharmaceutically acceptable salt thereof may be administered via an injection.

Yet, another embodiment relates to a method for preventing, treating or ameliorating an inflammation-mediated and/or complement-mediated disease or condition in a subject comprising administering to the subject a Fas inhibitor selected from the group consisting of Met protein, derivatives, fragments, pharmaceutically acceptable salts thereof; Met-12, derivatives, fragments, pharmaceutically acceptable salts thereof; SEQ ID NOs: 1-8, derivatives, fragments, pharmaceutically acceptable salts thereof; or a gene therapy agents encoding the Fas inhibitor, in an amount effective to inhibit Fas signaling, and thereby prevent, treat or ameliorate the inflammation-mediated and/or complement-mediated disease or condition in the subject. The subject has or is at risk of having the inflammation-mediated and/or complement-mediated disease or condition. The inflammation-mediated and/or complement-mediated disease or condition may be retinal disease (e.g., glaucoma, retinal detachment, AMD (dry and wet), diabetic retinopathy, Uveitis, retinal vein occlusion, inherited retinal degenerations, including retinitis pigmentosa, or NAION), immunological disease, cancer, amyloid disease (e.g., Alzheimer's disease, type-2 diabetes, Huntington's disease, ALS, or Parkinson's disease), an injury caused by ischemia or reperfusion (e.g., stroke), autoimmune disease (e.g., allergy, lupus, or rheumatoid arthritis), neurodegeneration, and diseases of the central nervous system (e.g., neuropathy or a demyelinating disease selected from the group consisting of multiple sclerosis and inflammatory demyelinating diseases). The Fas inhibitor may be administered in a pharmaceutical composition comprising the Fas inhibitor and a pharmaceutically acceptable additive selected from the group consisting of carriers, excipients, disintegrators or disintegrating aids, binders, lubricants, coating agents, pigments, diluents, bases, dissolving agents or solubilizers, isotonic agents, pH regulators, stabilizers, propellants, and adhesives. The Fas inhibitor may be administered via an injection (e.g., an intravitreal injection, intrathecal, intravenous, or periocular injection).

Yet, another embodiment relates to a method for preserving retinal ganglion cells and axon density, or preventing the loss of ganglion cells and axon density in a patient with glaucoma comprising administering to the subject a Fas inhibitor, a derivative thereof, a fragment thereof, a pharmaceutically acceptable salt thereof, or a gene therapy encoding the Fas inhibitor, wherein the preserving or preventing the loss of retinal ganglion cells and axon density, or preventing the loss thereof is due to at least one (or at least two, or all three) of the following: inhibition of microglial/macrophage activation or recruitment; inhibition of at least one of TNF-α, CCL2/MCP-1 or CCL3/MIP-1α gene or protein expression or concentration; or reduction of IL-1β gene or protein expression or protein maturation, wherein the Fas inhibitor is administered to the subject in an amount effective to inhibit Fas signaling. The Fas inhibitor, a derivative thereof, a fragment thereof, a pharmaceutically acceptable salt thereof, or a gene therapy encoding the Fas inhibitor may be administered in a pharmaceutical composition comprising the Fas inhibitor, a derivative thereof, a fragment thereof, a pharmaceutically acceptable salt thereof, or a gene therapy encoding the Fas inhibitor; and a pharmaceutically acceptable additive. The additive may be selected from the group consisting of carriers, excipients, disintegrators or disintegrating aids, binders, lubricants, coating agents, pigments, diluents, bases, dissolving agents or solubilizers, isotonic agents, pH regulators, stabilizers, propellants and adhesives. The composition may be in a form selected from the group consisting of: solution, pill, ointment, suspension, eye drops, gel, cream, foam, spray, liniment, and powder. The administering may be via an injection, wherein the injection is an intravitreal injection, intrathecal, intravenous or periocular injection. The composition may further comprise at least one non-ionic surfactant selected from the group consisting of Polysorbate 80, Polysorbate 20, Poloxamer 407, and Tyloxapol. The Fas inhibitor or the composition comprising the Fas inhibitor may be administered daily, twice daily, every other day, weekly, biweekly, monthly, bimonthly, or tri-monthly. The Fas inhibitor or the composition comprising Fas inhibitor may be administered in a daily dose of from about 1 ng to about 1 mg. The composition may be in the form of eye drops and the Fas inhibitor is in a concentration between 0.000001% w/v and 2% w/v.

Yet, another embodiment relates to a method of treating a subject having at least a 10% increase in the mRNA and/or protein expression level(s) of at least one (or at least two, or at least three, or at least four, etc., or all) of the following gene and/or protein in the subject's eye, as compared to a control: at least one Fas-mediated inflammation-related gene or protein (e.g. TNFα, IL-1β, IP-10, IL-18, MIP1α, IL-6, GFAP, MIP2, MCP-1, or MIP-1β); at least one Fas-mediated complement-related gene or protein (complement component 3 (C3) or complement component 1q (C1q)); Caspase 8; one or more components of the inflammasome (e.g., NLRP3 or NLRP2); one or more C-X-C motif chemokines (e.g., CXCL2 (MIP-2α) or CXCL10 (IP-10)); one or more C-X3-C motif chemokines (e.g., CX3CL1 (fractalkine)); one or more C-C motif chemokines (CCL2 (MCP-1), CCL3 (MIP-1α), and CCL4 (MIP-1β)); toll-like receptor 4 (TLR4); one or more interleukin cytokines (e.g., IL-1β, IL-18, and IL-6); one or more TNF superfamily cytokines (e.g., TNFα); or GFAP gene or protein expression or concentration, the method comprising administering to the subject a Fas inhibitor. The Fas inhibitor may be any Fas inhibitor described herein. For example, the Fas inhibitor may be selected from the group consisting of: Met protein, derivatives, fragments, pharmaceutically acceptable salts thereof; Met-12, derivatives, fragments, pharmaceutically acceptable salts thereof; SEQ ID NOs: 1-8, derivatives, fragments, pharmaceutically acceptable salts thereof; or a gene therapy agents encoding the Fas inhibitor.

Yet, a further embodiment relates to a method of treating a subject having at least a 5% increase in the mRNA and/or protein expression level(s) of at least one (or at least two, or at least three, or at least four, etc., or all) of the following gene and/or protein in the subject's serum, plasma, whole blood, or cerebrospinal fluid, as compared to a control: at least one Fas-mediated inflammation-related gene or protein (e.g. TNFα, IL-1β, IP-10, IL-18, MIP1α, IL-6, GFAP, MIP2, MCP-1, or MIP-1β); at least one Fas-mediated complement-related gene or protein (complement component 3 (C3) or complement component 1q (C1q)); Caspase 8; one or more components of the inflammasome (e.g., NLRP3 or NLRP2); one or more C-X-C motif chemokines (e.g., CXCL2 (MIP-2α) or CXCL10 (IP-10)); one or more C-X3-C motif chemokines (e.g., CX3CL1 (fractalkine)); one or more C-C motif chemokines (CCL2 (MCP-1), CCL3 (MIP-1α), and CCL4 (MIP-1β)); toll-like receptor 4 (TLR4); one or more interleukin cytokines (e.g., IL-1β, IL-18, and IL-6); one or more TNF superfamily cytokines (e.g., TNFα); or GFAP gene or protein expression or concentration, the method comprising administering to the subject a Fas inhibitor, the method comprising administering to the subject a Fas inhibitor. The Fas inhibitor may be any Fas inhibitor described herein. For example, the Fas inhibitor may be selected from the group consisting of: Met protein, derivatives, fragments, pharmaceutically acceptable salts thereof; Met-12, derivatives, fragments, pharmaceutically acceptable salts thereof; SEQ ID NOs: 1-8, derivatives, fragments, pharmaceutically acceptable salts thereof; or a gene therapy agents encoding the Fas inhibitor.

Yet, a further embodiment relates to a composition comprising a compound selected from the group consisting of Compounds 2-8, a derivative thereof, an analog thereof, or a fragment thereof.

### BRIEF DESCRIPTION OF THE DRAWINGS

FIG. 1 shows a graph depicting blockade of Fas-induced caspase 8 activation by Met 12 and Compound 1 trihydrochloride in 661W cells. 661W cells were, pre-treated with various amounts of either Met-12 or Compound 1 dissolved in DMSO, both at 20 mg/mL for 1 hr Then FasL (500 ng/mL) was added, and Caspase 8 activity was measured at 48 hours after treatment with FasL.

FIG. 2 shows a graph depicting blockade of Fas-induced caspase 8 activation by Met-12 and Compound 1 trihydrochloride in 661W cells. 661W cells were pretreated for 1 hr with various amounts of Met-12 in DMSO (circle), Compound 1 in DMSO (20 mg/mL diamond) and Compound 1 in a 2% Polysorbate (PS) 20, 2% propylene glycol (PG) pH 4 formulation (triangle), all formulations at a 10 mg/mL concentration. The cells were then treated with FasL (500 ng/mL) and Caspase 8 activity was measured at 48 hours after treatment with FasL.

FIG. 3 shows a graph depicting blockade of Fas-induced caspase 8 activation by Compound 1 trihydrochloride in 661W cells. 661W cells were pretreated for 1 hr with various amounts of Compound 1 in DMSO (20 mg/mL) (circle), and in a 3% Polysorbate 20, 3% propylene glycol pH 4 formulation (triangle), and in a 1% Polysorbate 20, 3% propylene glycol pH 4 formulation (diamond), all formulations at a 10 mg/mL concentration. The cells were then treated with FasL (500 ng/mL) and Caspase 8 activity was measured at 48 hours after treatment with FasL.

FIG. 4 shows a graph depicting blockade of Fas-induced caspase 8 activation by Compound 1 trihydrochloride in 661W cells. 661W cells were pretreated with various amounts of Compound 1 trihydrochloride in DMSO (20 mg/mL) (circle), and in an 0.4% Polysorbate-20, 4.5% mannitol, 10 mM acetate pH 4 formulation (triangle), at a 2 mg/mL concentration. The cells were then treated with FasL (500 ng/mL) and Caspase 8 activity was measured at 48 hours after treatment with FasL.

FIG. 5a depicts a logarithmic graph of rabbit retina concentrations of Compound 1 delivered intravitreally in three Poloxamer formulations.

FIG. 5b depicts a logarithmic graph of rabbit retina concentrations of Compound 1 delivered intravitreally to compare a Poloxamer 407-based formulation with a Polysorbate-20 based formulation over time.

FIG. 6a depicts linear graph of rabbit vitreous humor (VH) concentrations over time of Compound 1 delivered intravitreally in three Poloxamer formulations with varying concentrations of surfactant (0.4% or 0.1%) and varying concentrations of Compound 1 (2 mg/mL vs 0.5 mg/mL).

FIG. 6b depicts a linear graph of rabbit VH concentrations over time with different choice of surfactant (0.4% Poloxamer 407 vs. 0.4% Polysorbate 20) and varying amount of Compound 1 (2 mg/mL vs 1 mg/mL).

FIG. 7 shows total amounts of Compound 1 triacetate in the vitreous humor, (dark) and concentrations in the retinas, (light) of brown Norway rats 24 and 72 hours after a nominal injection of 300 ng of Compound 1 triacetate (5 μL of 0.06 mg/mL) in 4.5% mannitol, 10 mM acetic acid, 0.4% poloxamer (PX) 407, pH 4.5.

FIG. 8 shows a bar graph depicting the number of apoptotic cells after 72 hrs following in vivo treatments of detached retinas in rats with Met-12 trihydrochloride (light stripe) (5 μg in 5 μL DMSO), and Compound 1 trihydrochloride (dark) (0.5, 1.0, 5 and 10 μg in 5 μL DMSO), or DMSO vehicle (light). The LHS bar is undetached control retina with no injection.

FIG. 9 shows a bar graph depicting the number of apoptotic cells after 72 hrs following in vivo treatments of detached retinas in rats with Compound 1 trihydrochloride (1.0 and 5 μg in 5 μL of F1 or F2), against the same (5 μg in 5 μL) in DMSO or DMSO vehicle (gray). The LHS bar is undetached control retina with no injection. F1 (5/1 μg) is 1.0/0.2 mg/mL Compound 1 trihydrochloride in 3% PG/3% PS-20 at pH 4.0 (black), and F2 (5/1 μg) is 1.0/0.2 mg/mL Compound 1 trihydrochloride in 2% PG/2% PX-407 at pH 4.0 (vertical stripes).

FIG. 10 shows a bar graph depicting the percent of apoptotic cells after 72 hrs following in vivo treatments of detached retinas in rats. Bar 1 is a vehicle control in detached retinas. Bar 2 with 1 μg of Compound 1 triacetate as an 0.2 mg/mL DMSO solution. Bar 3 is 1 μg of Compound 1 trihydrochloride as an 0.2 mg/mL DMSO solution. Bar 4 is undetached control retina with no injection.

FIG. 11 depicts bar graphs showing the expression of the inflammation-related genes: (A) TNF, (B) IL-1β, (C) IP-10, (D) IL-18, (E) MIP-1α, (F) IL-6, (G) GFAP, (H) MIP2, and (i) Complement C3 in samples treated with Compound 1, as compared to the vehicle and microbeads alone.

FIG. 12 depicts bar graphs showing the expression of genes: (A) MCP-1, (B) Caspase 8, (C) CFLIP, (D) TLR-4, (E) MIP-1β, (F) NLRP3, and (G) Complement C1Q in samples treated with Compound 1, as compared to the vehicle and microbeads alone.

FIG. 13 depicts bar graphs showing the expression of genes: (A) Bax, (B) FADD, (C) ASC, (D) FasR, (E) FasL, (F) Complement C4, (G) NLRP2, and (H) Caspase 3 in samples treated with Compound 1, as compared to the vehicle and microbeads alone.

FIG. 14 depicts IOP graph for the study with drug/vehicle given at the same time as microbeads.

FIG. 15 depicts IOP graph for the study with drug/vehicle injection 7 days post-injection of microbeads.

FIG. 16 depicts representative images from RGC and axon counts for drug/vehicle injected at the same time as microbeads/saline.

FIG. 17 depicts a bar graph based on the quantification of the collected images for RGC cell density.

FIG. 18 depicts a bar graphs based on the quantification of the collected images for axon density.

FIG. 19 depicts representative images from RGC and axon data for the day 7 drug/vehicle injection study.

FIG. 20 depicts a bar graph based on the quantification of the collected images for RGC cell density for day 7 drug/vehicle injection study.

FIG. 21 depicts a bar graph based on the quantification of the collected images for axon density for day 7 drug/vehicle injection study.

FIG. 22 depicts images showing that treatment with Compound 1 inhibits the activation of retinal microglia and/or the infiltration of macrophages into the retina following elevated IOP, and the quantification of process length of the microglia (bar graph).

FIG. 23 depicts a bar graph for Western blot analysis following microbead injection in the mice treated with Compound 1 as compared to vehicle.

### DETAILED DESCRIPTION

All patents, patent applications and publications, and other literature references cited herein are hereby incorporated by reference in their entirety. The disclosures of these publications in their entireties are hereby incorporated by reference into this application in order to more fully describe the state of the art as known to those skilled therein as of the date of the invention described and claimed herein.

Biologically active peptide compositions, pharmaceutical preparations of biologically active peptide compositions, and methods of using the peptide compositions are described.

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, compositions, devices and materials are described herein.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and,” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The terms “optional” or “optionally” mean that the subsequently described event, circumstance, or component may but need not occur, and that the description includes instances where the event or circumstance occurs and instances in which it does not.

As used herein, the term “about” modifying, for example, the quantity of an ingredient in a composition, concentration, volume, process temperature, process time, yield, flow rate, pressure, and like values, and ranges thereof, employed in describing the embodiments of the disclosure, refers to variation in the numerical quantity that can occur, for example, through typical measuring and handling procedures used for making compounds, compositions, concentrates or use formulations; through inadvertent error in these procedures; through differences in the manufacture, source, or purity of starting materials or ingredients used to carry out the methods, and like proximate considerations. The term “about” also encompasses amounts that differ due to aging of a formulation with a particular initial concentration or mixture, and amounts that differ due to mixing or processing a formulation with a particular initial concentration or mixture. Where modified by the term “about” the claims appended hereto include equivalents to these quantities. Further, where “about” is employed to describe a range of values, for example “about 1 to 5” the recitation means “1 to 5” and “about 1 to about 5” and “1 to about 5” and “about 1 to 5,” unless specifically limited by context.

The term “therapeutically effective amount” means an amount of a drug or agent (e.g., Compound 1) effective to facilitate a desired therapeutic effect in a particular class of subject (e.g., infant, child, adolescent, adult). As used herein, the term “subtherapeutic” refers to an amount of a pharmaceutical drug or agent that is insufficient to achieve the desired and/or anticipated therapeutic result/outcome upon administration to an average and/or typical subject (e.g., average size, taking no contraindicated pharmaceutical agents, having a similar reaction to the dose as a majority of the population, etc.). U.S. Food and Drug Administration (FDA) recommended dosages are indicative of a therapeutic dose.

As used herein, the terms “pharmaceutical drug” or “pharmaceutical agent” refer to a compound, peptide, macromolecule, or other entity that is administered (e.g., within the context of a pharmaceutical composition) to a subject to elicit a desired biological response. A pharmaceutical agent may be a “drug” or any other material (e.g., peptide, polypeptide), which is biologically active in a human being or other mammal, locally and/or systemically. Examples of drugs are disclosed in the Merck Index and the Physicians Desk Reference, the entire disclosures of which are incorporated by reference herein for all purposes.

As used herein, the term “pharmaceutical formulation” refers to at least one pharmaceutical agent (e.g., Compound 1) in combination with one or more additional components that assist in rendering the pharmaceutical agent(s) suitable for achieving the desired effect upon administration to a subject. The pharmaceutical formulation may include one or more additives, for example pharmaceutically acceptable excipients, carriers, penetration enhancers, coatings, stabilizers, buffers, acids, bases, or other materials physically associated with the pharmaceutical agent to enhance the administration, release (e.g., timing of release), deliverability, bioavailability, effectiveness, etc. of the dosage form. The formulation may be, for example, a liquid, a suspension, a solid, a nanoparticle, emulsion, micelle, ointment, gel, emulsion, coating, etc. A pharmaceutical formulation may contain a single pharmaceutical agent (e.g., Compound 1) or multiple pharmaceutical agents. A pharmaceutical composition may contain a single pharmaceutical formulation or multiple pharmaceutical formulations. In some embodiments, a pharmaceutical agent (e.g., Compound 1) is formulated for a particular mode of administration (e.g., ocular administration (e.g., intravitreal administration, etc.), etc.). A pharmaceutical formulation is sterile, non-pyrogenic and non-toxic to the eye.

As used herein, the term “pharmaceutical composition” refers to the combination of one or more pharmaceutical agents with one or more carriers, inert or active, making the composition especially suitable for diagnostic or therapeutic use in vitro, in vivo or ex vivo. A pharmaceutical composition comprises the physical entity that is administered to a subject, and may take the form of a solid, semi-solid or liquid dosage form, such as tablet, capsule, orally-disintegrating tablet, pill, powder, suppository, solution, elixir, syrup, suspension, cream, lozenge, paste, spray, etc. A pharmaceutical composition may comprise a single pharmaceutical formulation (e.g., extended release, immediate release, delayed release, nanoparticulate, etc.) or multiple formulations (e.g., immediate release and delayed release, nanoparticulate and non-nanoparticulate, etc.). The terms “pharmaceutical composition” and “pharmaceutical formulation” may be used interchangeably.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants see, e.g., Martin, Remington's Pharmaceutical Sciences, 15th Ed., Mack Publ. Co., Easton, Pa. [1975]; herein incorporated by reference in its entirety.

As used herein, the term “pharmaceutically acceptable salt” refers to any acid or base of a pharmaceutical agent or an active metabolite or residue thereof. As is known to those of skill in the art, “salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

As used herein, the term “administration” refers to the act of giving a drug, prodrug, or other agent, or therapeutic treatment (e.g., compositions of the present invention) to a subject (e.g., a subject or in vivo, in vitro, or ex vivo cells, tissues, and organs). Exemplary routes of administration to the human body can be through the eyes (ophthalmic), mouth (oral), skin (transdermal), nose (nasal), lungs (inhalant), oral mucosa (buccal), ear, rectal, by injection (e.g., intravenously, subcutaneously, intratumorally, intraperitoneally, etc.) and the like.

As used herein, the term “co-administration” refers to the administration of at least two agent(s) (e.g., Compound 1 and one or more additional therapeutics) or therapies to a subject. In some embodiments, the co-administration of two or more agents/therapies is concurrent. In other embodiments, the co-administration of two or more agents/therapies is sequential (e.g., a first agent/therapy is administered prior to a second agent/therapy). In some embodiments, the two or more therapies are administered concurrently, but released (e.g., absorbed, become bioavailable, etc.) sequentially. Those of skill in the art understand that the formulations and/or routes of administration of the various agents/therapies used may vary. The appropriate dosage for co-administration can be readily determined by one skilled in the art. In some embodiments, when agents/therapies are co-administered, the respective agents/therapies are administered at lower dosages than appropriate for their administration alone.

As used herein, “treatment” refers to a clinical intervention made in response to a disease, disorder or physiological condition manifested by a patient or to which a patient may be susceptible. The aim of treatment includes, but is not limited to, the alleviation or prevention of symptoms, slowing or stopping the progression or worsening of a disease, disorder, or condition and/or the remission of the disease, disorder or condition. “Treatments” refer to one or both of therapeutic treatment and prophylactic or preventative measures. Subjects in need of treatment include those already affected by a disease or disorder or undesired physiological condition as well as those in which the disease or disorder, or undesired physiological condition is to be prevented. In certain embodiments, treatment refers to the alleviation or prevention of symptoms, slowing or stopping the progression or worsening of an inflammation-mediated and/or complement-mediated pathology and/or tissue damage in a disease, disorder, or condition to be treated with Fas inhibitors, as described in detail below, and/or the remission of the disease, disorder or condition.

The term “express” and “expression” means allowing or causing the information in a gene or DNA sequence to become manifest, for example producing RNA (such as rRNA or mRNA) or a protein by activating the cellular functions involved in transcription and translation of a corresponding gene or DNA sequence. The term “reduction of expression or concentration” refers to a decrease in production or amount of the specified gene or protein. The term “gene,” means a DNA sequence that codes for or corresponds to a particular sequence of amino acids, which comprise all or part of one or more proteins or enzymes, and may or may not include regulatory DNA sequences, such as promoter sequences, which determine for example the conditions under which the gene is expressed. Some genes, which are not structural genes, may be transcribed from DNA to RNA, but are not translated into an amino acid sequence. Other genes may function as regulators of structural genes or as regulators of DNA transcription.

As used herein, a “subject” or “patient” refers to an animal that is the object of treatment, observation or experiment. “Animal” includes cold- and warm-blooded vertebrates and invertebrates such as fish, shellfish, reptiles, and in particular, mammals. “Mammal,” as used herein, refers to an individual belonging to the class Mammalia and includes, but not limited to, humans, domestic and farm animals, zoo animals, sports and pet animals. Non-limiting examples of mammals include mice; rats; rabbits; guinea pigs; dogs; cats; sheep; goats; cows; horses; primates, such as monkeys, chimpanzees and apes, and, in particular, humans. In some embodiments, the mammal is a human. However, in some embodiments, the mammal is not a human.

A “control” is an alternative subject or sample used in an experiment for comparison purposes. A control can be “positive” or “negative.” For example, where the purpose of the experiment or the comparison in a method is to determine a correlation of an patient treatment with a particular symptom, one may use either a positive control (a patient exhibiting the symptom and not subjected to the treatment, or a sample from such a patient), and/or a negative control (a subject that does not exhibit the symptom and not subjected to the treatment, or a sample from such a subject).

The term “reduced” or “reduce” as used herein generally means a decrease by at least 5% as compared to a reference or control level, for example, a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease, or any integer decrease between 10-100% as compared to a reference or control level.

The term “increased” or “increase” as used herein generally means an increase of at least 5% as compared to a reference or control level, for example an increase of at least 10% as compared to a reference level, or at least about 20%, or at least about 30%, or at least about 40%, or at least about 50%, or at least about 60%, or at least about 70%, or at least about 80%, or at least about 90% or up to and including a 100% increase or any integer increase between 10-100% as compared to a reference level, or about a 2-fold, or about a 3-fold, or about a 4-fold, or about a 5-fold or about a 10-fold increase, or any increase between 2-fold and 10-fold or greater as compared to a reference or control level.

Provided herein are pharmaceutical preparations of biologically active, aqueous formulations of a photoreceptor-protective peptide, pharmaceutical preparations thereof, and methods of preventing photoreceptor death therewith as well as therapeutic methods.

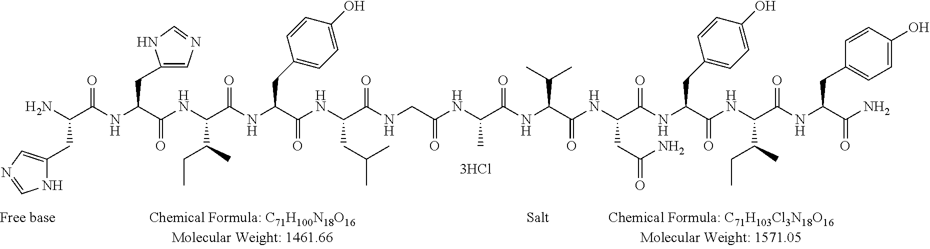 [SMILES: CC[C@H](C)[C@H](NC(=O)[C@H](Cc1ccc(O)cc1)NC(=O)[C@H](CC(N)=O)NC(=O)[C@@H](NC(=O)[C@H](C)NC(=O)CNC(=O)[C@H](CC(C)C)NC(=O)[C@H](Cc1ccc(O)cc1)NC(=O)[C@@H](NC(=O)[C@H](Cc1cnc[nH]1)NC(=O)[C@@H](N)Cc1cnc[nH]1)[C@@H](C)CC)C(C)C)C(=O)N[C@@H](Cc1ccc(O)cc1)C(N)=O]

Compound I.
(SEQ ID NO: I)
His-His-Ile-Tyr-Leu-Gly-Ala-Val-Asn-Tyr-Ile-Tyr-
amide;
Formula I.

Some embodiments relate to a C-terminal amide peptide, Compound 1 (above), or a pharmaceutically acceptable salt thereof. Certain embodiments relate to a polyacetate salt of the Compound 1. Certain further embodiments relate to a triacetate salt of the Compound 1.

The compounds may be for use in a pharmaceutical formulation for preventing Fas- or TRAIL mediated apoptosis in the photoreceptors of the eye. In a FasL-induced model of photoreceptor toxicity, in 661W cells, Compound 1 is 10-fold more potent at preventing Caspase 8 activation than Met-12 by IC₅₀, and approximately 3-fold more potent than Met-12 measured by dose potency at maximal inhibition. In an in vivo rat model of retinal detachment, Compound 1 is at least 10-fold more potent than is Met-12 at protecting photoreceptor cells from apoptosis, and, unlike Met-12 can be delivered efficaciously in clinically acceptable formulations.

As demonstrated in the examples, Fas inhibition by Compound 1 resulted in significant preservation of photoreceptor cells in vivo. In 661W cells, Compound 1 treatment resulted in profound inhibition of the caspase 8 activation. As such, it is believed that administration of Compound 1 to a subject with an ocular condition, disease, or condition or disease affecting ocular health may yield improved protection of retinal cells including, but not limited to, photoreceptors, retinal pigment epithelium cells and retinal ganglion cells, from Fas-mediated apoptosis, resulting in improvement and/or treatment of the ocular condition, disease, or condition or disease affecting ocular health.

In clinical practice, patients generally present with a detachment having already occurred. The animal models of retina-RPE separation show that Fas-pathway activation takes place early and remains elevated throughout the duration of the detachment (Zacks et al. Arch Ophthalmol 2007; 125:1389-1395, Zacks et al. IOVS 2004; 45(12):4563-4569.8.). The separation of retina and RPE is also encountered in a broad spectrum of retinal diseases. It is contemplated that the clinical relevance of anti-Fas therapy in retinal cell survival is not limited to retinal detachment. For example, Fas-mediated apoptosis may play a role in photoreceptor cell death in age-related macular degeneration (AMD) (Dunaief et al. Arch Ophthalmol. 2002; 120(11):1435-1442; Zacks et al. Arch Ophthalmol 2007; Petrukhin K. New therapeutic targets in atrophic age-related macular degeneration. Expert Opin Ther Targets. 2007. 11:625-639; Miller J W. Treatment of age-related macular degeneration: beyond VEGF. Jpn J Ophthalmol. 2010. 54:523-528; Rogala J, Zangerl B, Assaad N, Fletcher E L, Kalloniatis M, Nivison-Smith L. In Vivo Quantification of Retinal Changes Associated with Drusen in Age-Related Macular Degeneration. Invest Ophthalmol Vis Sci. 2015. 56:1689-1700, herein incorporated by reference in its entirety). Age-related macular degeneration is characterized by progressive degeneration of the RPE and causes outer retinal degeneration and re-organization similar to that which occurs after retinal detachment (Jager et al. N Engl J Med. 2008; 358:2606-17, Johnson et al. Invest Ophthalmol Vis Sci. 2003; 44:4481-488, herein incorporated by reference in their entireties). In the neovascular form of AMD there is also the exudation of fluid under the retina, creating an actual separation of this tissue from the underlying RPE (Jager et al. N Engl J Med. 2008; 358:2606-17, herein incorporated by reference in its entirety). Neovascular AMD can result in prolonged periods of retina-RPE separation and Fas-pathway activation. The utility of anti-Fas treatment would most likely be as an adjunct aimed at protecting retinal cells (such as photoreceptors and retinal pigment epithelium) while the underlying disorder is being treated (Brown et al. N Engl J Med. 2006 Oct. 5; 355(14):1432-44, herein incorporated by reference in its entirety).

Glaucoma is a progressive degenerative ocular condition that is characterized by the death of the retinal ganglion cells (RGCs), and previously published research has demonstrated that the RGCs die by apoptosis (Ji et al. Vision Res. 2005; 45(2): 169-179). Intraocular pressure (IOP) is a major risk factor for glaucoma development and substantial efforts have been devoted to reducing IOP using prostaglandin analogs in order to prevent RGC apoptosis (Doucette and Walter. Ophthalmic Genet. 2016; 12: 1-9). Fas has also been implicated in RGC death (Gregory et al. PLoS One. 2011; 6(3):e17659), and animal models of IOP exhibit increased Fas and FasL expression (Ju et al. Brain Res. 2006; 1122(1): 209-221), indicating the potential utility of Fas inhibition as a means to protect RGC viability and mitigate the degenerative nature of glaucoma.

In some embodiments, the described polypeptide can be prepared by methods known to those of ordinary skill in the art. For example, the claimed Compound 1 can be synthesized using standard solid phase polypeptide synthesis techniques (e.g., Fmoc). Alternatively, the polypeptide can be synthesized using recombinant DNA technology (e.g., using bacterial or eukaryotic expression systems), which overexpress both the peptide and an appropriate amidase enzyme to carry out the C-terminal amidation.

Specifically, as described in Example 1, Compound 1 can be obtained by building the Met-12 peptide sequence, H⁶⁰HIYLGATNYIY⁷¹ (SEQ ID NO: 2) onto an amino resin, as is known to those of skill in the art to produce after deprotection and resin cleavage its C-terminal amide H⁶⁰HIYLGATNYIY⁷¹-NH₂, Compound 1 (SEQ ID NO: 1). Specifically, Compound 1 can be obtained conceptually from the c-Met sequence by a normal amide hydrolysis between residues 59 and 60, and an unnatural breaking of the peptide chain between the peptide nitrogen and the α-carbon of residue 72, rather than at the carbonyl carbon of residue 71. This is not a cleavage, which occurs naturally. Met-12 has been previously described in U.S. Pat. No. 8,343,931, which is incorporated herein in its entirety.

The use of a C-terminally amidated peptide, i.e., Compound 1, was based on a belief that this specific modification might raise the pH at which the peptide is soluble in water or miscible in micelles by removal of the free carboxylic acid, which is significantly deprotonated above pH 3. The resulting species would not have a C-terminal anion at any physiologically relevant pH, or be a zwitterion under any physically relevant circumstances, and would be a tricationic species below about pH 5. This alteration could be most readily achieved by conversion into an amide or ester, neither of which is deprotonatable under physiological conditions. Amides are more biologically and chemically stable than esters, and also less hydrophobic, so the simple primary amide was chosen.

In certain embodiments, Compound 1 can be produced by converting Met-12 into its C-terminal primary amide, to form Compound 1, although it is generally more practical to build up the peptide from an already aminated first amino acid residue, by use of an amino resin, familiar to one of skill in the art. As noted in the examples section below, Compound 1 was obtained and tested originally as a trihydrochloride, although later a triacetate salt was deemed more advantageous for formulation.

There are certain advantages of using Compound 1 over Met-12. Specifically, as shown in the examples below, Compound 1 can be formulated with surfactants to produce micellar solutions at pHs and additive amounts, which are precedented in ocular formulations. Second, based on the in vitro efficacy assay, Compound 1 is surprisingly 10-fold more potent than Met-12 by IC₅₀ determination and approximately 3-fold more potent measured by concentration of maximal inhibition. Specifically, when Met-12 and Compound 1 are tested in the same formulation in vitro, Compound 1 has greater dose potency than Met-12. This allows for the same physiological effect to be achieved with lower amounts of Compound 1 than of Met-12. Third, in in vivo testing in a rat model of retinal detachment, Compound 1 surprisingly is at least five times as potent as Met-12 in preventing apoptosis in photoreceptor cells in the detached portion of the retina. Fourth, in some of the disclosed formulations of Compound 1, efficacy in the rat retinal detachment model is achieved at levels more than 10-fold lower than seen with Met-12. Finally, Compound 1 shows very extended half lives in both vitreous humor, and retinas of rabbits treated intravitreally, and these half lives can be extended to different extents by using different formulations, allowing the overall retinal exposure to Compound 1 to be controlled by the formulation chosen.

In some embodiments, Compound 1 is effective in one or more of: preventing/inhibiting/reducing Fas-mediated photoreceptor apoptosis, preventing apoptosis in cells of the retinal pigmented epithelium of the eye, increasing photoreceptor survival, preventing cell death related to age-related macular degeneration (AMD), preventing cell death related to retinal detachment, etc. In some additional embodiments, Compound 1 is effective in protecting retinal ganglion cells, which receive visual information from photoreceptors via two intermediate neuron types: bipolar cells and retina amacrine cells.

In some embodiments, a therapeutically active amount of Compound 1 or preparation thereof (i.e., a formulation or a composition) is administered to a mammalian subject in need of treatment (e.g., for a particular ocular condition) and at a location sufficient to inhibit or attenuate apoptosis within the patient (e.g., within desired tissue). The preferred subject is a human with an ocular condition, disease, or condition or disease affecting ocular health.

The amount administered is sufficient to yield improved protection of retinal cells and/or retinal ganglion cells, including, but not limited to, photoreceptors, retinal pigment epithelium and retinal ganglia, from Fas-mediated apoptosis, or prevent retinal cell death, resulting in improvement and/or treatment of the ocular condition, disease, or condition or disease affecting ocular health.

The determination of a therapeutically effective dose is within the capability of practitioners in this art. In some embodiments, an effective human dose will be in the range of 5-10,000 μg/eye, 50-5,000 μg/eye, or 100-2,000 μg/eye. Repeated doses are contemplated in order to maintain an effective level (e.g., weekly, every other week, monthly, quarterly, semi-annually etc.).

In some embodiments, a pharmaceutical formulation is a sterile, non-pyrogenic liquid and comprises at least 0.1 mg/ml (e.g., >0.1, >0.2, >0.5, >0.6, >0.7, >0.8, and >0.9), at least 1 mg/ml (e.g., >1 mg/ml, >2 mg/ml, >5 mg/ml, >10 mg/ml, etc.) of a peptide/polypeptide described herein (e.g., 1 mg/ml, 2 mg/ml, 5 mg/ml, 10 mg/ml, or more) of a peptide/polypeptide (e.g., Compound 1).

In some embodiments, a therapeutic dose comprises at least 0.01 ml (e.g., 0.01 ml . . . 0.02 ml . . . 0.05 ml . . . 0.1 ml . . . 0.2 ml . . . 0.5 ml . . . 1 ml . . . 2 ml . . . 3 ml . . . 4 ml, and volumes and ranges therein) of a liquid pharmaceutical formulation comprising a photoreceptor- or RPE-protective peptide/polypeptide (e.g., Compound 1). In some embodiments, a liquid volume of 10 to 500 μl is injected into the human eye (e.g., 10 μl, 20 μl, 30 μl, 40 μl, 50 μl, 75 μl, 100 μl, 200 μl, 300 μl, 400 μl, 500 μl, and volumes and ranges therein). In some embodiments, a volume of 50 to 600 μl is injected into the human eye (e.g., 50 μl, 75 μl, 100 μl, 200 μl, 300 μl, 400 μl, 500 μl, 600 μl, and volumes and ranges therein). In some embodiments, when injected intra-operatively milliliter scale volumes may be used (e.g., up to the total volume of the vitreous cavity (e.g., about 4 ml). In some embodiments the compound may be incorporated into perfusate solution used for maintaining internal ocular pressure during a vitrectomy.

In some embodiments, a single dose is provided (e.g., to treat an acute condition (e.g., retinal detachment). In some embodiments, multiple doses (e.g., daily, weekly, monthly, etc.) are provided for treatment of a chronic condition. The formulation may be different depending on the needed duration of exposure for the condition being treated.

In some embodiments, treatment dosages are titrated upward from a low level to optimize safety and efficacy. In some embodiments for intravitreal injection, a dose includes 0.01 to 5 mg of peptide (e.g., 0.1 and 2.0 mg).

In some embodiments, pharmaceutical preparations (i.e., formulations and/or compositions) comprise one or more excipients. Excipients suitable for ocular application, include, but are not limited to, tonicity agents, preservatives, chelating agents, buffering agents, surfactants, cosolvents and antioxidants. Suitable tonicity-adjusting agents include mannitol, sodium chloride, glycerin, sorbitol and the like. Suitable preservatives include p-hydroxybenzoic acid ester, benzalkonium chloride, benzododecinium bromide, polyquaternium-1 and the like. Suitable chelating agents include sodium edetate and the like. Suitable buffering agents include phosphates, borates, citrates, acetates, tromethamine, and the like. Suitable surfactants include ionic and nonionic surfactants, though nonionic surfactants are preferred, such as polysorbates, polyethoxylated castor oil derivatives, polyethoxylated fatty acids, polyethoxylated alcohols, polyoxyethylene-polyoxypropylene block copolymers (Poloxamer), and oxyethylated tertiary octylphenol formaldehyde polymer (Tyloxapol). Other suitable surfactants may also be included. Suitable antioxidants include sulfites, thiosulfate, ascorbates, BHA, BHT, tocopherols, and the like.

The compositions of the present invention optionally comprise an additional active agent. Such additional active agents might include anti-TNF antibodies, such as Adalimumab (Ophthalmic Surg Lasers Imaging Retina 45, 332 (2014), Curr Eye Res 39, 1106 (2014)) or etanercept (PLoS One, 7, e40065), or kinase inhibitors shown to preserve retinal structure such as the ROCK inhibitor Y-27632 (Molecular Medicine Reports 12, 3655 (2015)), the adenosine kinase inhibitor ABT-702 (Life Sci 93, 78 (2013), or the JNK inhibitory peptide D-JNK-1 (Diabetes 50, 77 (2001), Adv Exptl Med Biol 854, 677 (2016)), or docosahexaenoic acid (J Lipid Res, 54,2236 (2013)) or the RXR pan-agonist PA024 (ibid) or necrostatin, or RIP kinase inhibitors such as Dabrafenib. (Cell Death Dis 5, 1278 (2014))

In some exemplary embodiments, at least one of excipients, such as, Polysorbate 20 (e.g., up to 3%), Poloxamer 407 (e.g., up to 2%), Tyloxapol (e.g., up to 3%), cremophor (e.g., up to 1%); and/or cosolvents (e.g., between 0.5 and 50%), such as N,N-Dimethylacetamide, ethanol, PEG-400, propylene glycol, dimethylsulfoxide (DMSO); oils, or cyclodextrins may be added to a pharmaceutical preparation.

In further exemplary embodiments, at least one nonionic surfactant (e.g., 0.1%-20% w/w/of the composition), such as Polysorbate 80, Polysorbate 20, Poloxamer, or

Tyloxapol may be included in the pharmaceutical composition. In addition, an organic cosolvent, such as propylene glycol or dimethylsulfoxide in an amount of approximately 1-50%, may be included in the pharmaceutical composition. Alternatively, an organic cosolvent, such as N,N-Dimethylacetamide, ethanol, PEG-400, propylene glycol, DMSO in an amount of approximately 1-20%, may be included in the pharmaceutical composition. Alternatively, an organic cosolvent, such as propylene glycol or dimethylsulfoxide in an amount of approximately 1-5%, may be included in the pharmaceutical composition. Alternatively, an isotonicity agent such as mannitol, sorbitol, glucose or trehalose, or an inorganic salt such as sodium chloride may be included in the pharmaceutical composition, in amounts needed to bring the tonicity of the composition into the 250-400 mOsm/L range.

The pH of the composition may be in the 2.5-6.0 range. The pH may be controlled by an appropriate buffer and be in the 3.0-5.0 range or 3.5-4.5 range.

In another exemplary embodiment, at least one nonionic surfactant (e.g., 0.5%-10% w/w/of the composition), such as Polysorbate 80, Polysorbate 20, Poloxamer, or Tyloxapol may be included in the pharmaceutical composition. In addition, an organic cosolvent, such as propylene glycol or dimethylsulfoxide in an amount of approximately 1-50%, may be included in the pharmaceutical composition. Alternatively, an organic cosolvent, such as propylene glycol or dimethylsulfoxide in an amount of approximately 1-20%, may be included in the pharmaceutical composition. Alternatively, an organic cosolvent, such as N,N-Dimethylacetamide, ethanol, PEG-400, propylene glycol, DMSO in an amount of approximately 1-5%, may be included in the pharmaceutical composition. Alternatively, an isotonicity agent such as mannitol, sorbitol, glucose or trehalose, or an inorganic salt such as sodium chloride may be included in the pharmaceutical composition, in amounts needed to bring the tonicity of the composition into the 250-400 mOsm/L range. The pH of the composition may be in the 2.5-6.0 range. The pH may be controlled by an appropriate buffer and be in the 3.0-5.0 range or 3.5-4.5 range.

In yet further exemplary embodiment, at least one nonionic surfactant (e.g., 1%-3% w/w/of the composition), such as Polysorbate 80, Polysorbate 20, Poloxamer, or Tyloxapol may be included in the pharmaceutical composition. In addition, an organic cosolvent, such as propylene glycol or dimethylsulfoxide in an amount of approximately 1-50%, may be included in the pharmaceutical composition. Alternatively, an organic cosolvent, such as N,N-Dimethylacetamide, ethanol, PEG-400, propylene glycol, DMSO in an amount of approximately 1-20%, may be included in the pharmaceutical composition. Alternatively, an organic cosolvent, such as propylene glycol or dimethylsulfoxide in an amount of approximately 1-5%, may be included in the pharmaceutical composition. Alternatively, an isotonicity agent such as mannitol, sorbitol, glucose or trehalose, or an inorganic salt such as sodium chloride may be included in the pharmaceutical composition, in amounts needed to bring the tonicity of the composition into the 250-400 mOsm range. The pH of the composition may be in the 2.5-6.0 range. The pH may be controlled by an appropriate buffer and be in the 3.0-5.0 range or 3.5-4.5 range.

In some embodiments, the pharmaceutical composition may include Compound 1 or a pharmaceutically acceptable salt thereof, and Poloxamer 407 (e.g., 0.1-2% w/w/of the composition) in an aqueous medium having pH in the 3.0-6.0 range.

In some embodiments, the pharmaceutical composition may include Compound 1 or a pharmaceutically acceptable salt thereof, and Poloxamer 407 (e.g., 0.1-2% w/w/of the composition) in an aqueous medium buffered by sodium propanoate/propanoic acid or sodium acetate/acetic acid having a pH in the 4.0-5.0 range.

In some embodiments, the pharmaceutical composition may include Compound 1 or a pharmaceutically acceptable salt thereof, and Poloxamer 407 (e.g., 0.1-2% w/w/of the composition) in an aqueous medium buffered by sodium propanoate/propanoic acid or sodium acetate/acetic acid having a pH in the 4.0-5.0 range, and made isotonic by 3-5% mannitol.

In some further embodiment, the pharmaceutical composition may include Compound 1, or a pharmaceutically acceptable salt thereof, Polysorbate-20 (e.g., 0.1-3% w/w/of the composition), and propylene glycol (e.g., 3% w/w/of the composition) in an aqueous medium in the pH range of 3.0-6.0.

In certain further embodiments, the pharmaceutical composition may include Compound 1, or a pharmaceutically acceptable salt thereof, Polysorbate-20 (e.g., 0.1-3% w/w/of the composition), and propylene glycol (e.g., 3% w/w/of the composition) in an aqueous medium buffered by sodium acetate/acetic acid in the pH range of 4.0-5.0.

In some further embodiment, the pharmaceutical composition may include Compound 1, or a pharmaceutically acceptable salt thereof, Polysorbate-20 (e.g., 0.1-3% w/w/of the composition), and mannitol (e.g., 3-5% w/w/of the composition) in an aqueous medium in the pH range of 3.0-6.0.

In certain further embodiments, the pharmaceutical composition may include Compound 1, or a pharmaceutically acceptable salt thereof, Polysorbate-20 (e.g., 0.1-3% w/w/of the composition), and mannitol (e.g., 3-5% w/w/of the composition) in an aqueous medium buffered by sodium acetate/acetic acid in the pH range of 4.0-5.0.

In some embodiments, the pharmaceutical composition may include Compound 1 or a pharmaceutically acceptable salt thereof, and Poloxamer 407 (e.g., 0.1-2% w/w/of the composition) and Polysorbate 20 (e.g., 0.1-2% w/w/of the composition) in an aqueous medium having pH in the 3.0-6.0 range.

In some embodiments, the pharmaceutical composition may include Compound 1 or a pharmaceutically acceptable salt thereof, and Poloxamer 407 (e.g., 0.1-2% w/w/of the composition) and Polysorbate 20 (e.g., 0.1-2% w/w/of the composition) in an aqueous medium buffered by sodium propanoate/propanoic acid or sodium acetate/acetic acid having a pH in the 4.0-5.0 range.

In some embodiments, the pharmaceutical composition may include Compound 1 or a pharmaceutically acceptable salt thereof, and Poloxamer 407 (e.g., 0.1-2% w/w/of the composition) and Polysorbate 20 (e.g., 0.1-2% w/w/of the composition) in an aqueous medium buffered by sodium propanoate/propanoic acid or sodium acetate/acetic acid having a pH in the 4.0-5.0 range, and made isotonic by 3-5% mannitol.

In some embodiments, the pharmaceutical compositions as described above may include Compound 1, but not chloride as a counterion, with acetate being a preferred alternative. Such compositions may show superior properties to those containing chloride ion.

In some embodiments, the weight ratio of the peptide/polypeptide (e.g., Compound 1) is 1%-25% relative to the weight of the non-aqueous excipients in the pharmaceutical formulation, which is conversely 0.1-20% excipients, such as Poloxamer, Polysorbate 20, propylene glycol and mannitol.

This weight ratio of the peptide/polypeptide (e.g., Compound 1) relative to the weight of the pharmaceutical formulation may be at least about 0.1%, at least 0.5%, at least 1%, at least about 2%, at least about 3%.

The following two exemplary compositions, having an amount of each ingredient in the range indicated, will provide two of several compositions that may be used to treat or prevent various ocular diseases or conditions (e.g., of the retina) or preventing retinal cell death resulting from ocular diseases or conditions or the like in a subject:

Exemplary Formulation I:

| Compound 1 triacetate salt | 0.1-2 | mg/mL |
|---|---|---|
| Poloxamer 407 | 0.01-0.5% | |
| Additives (e.g., Mannitol) | 2.5-5% | |
| Acetic acid | 10 | mM |

| | |
|---|---|
| NaOH | To pH > 3 |
| Water (WFI) | To 100% |

Exemplary Formulation II:

| Compound 1 triacetate salt | 0.1-2 | mg/mL |
|---|---|---|
| Polysorbate 20 | 0.1-1.0% | |
| Additives (e.g., Mannitol) | 2.5-5% | |
| Acetic acid | 10 | mM |

| | |
|---|---|
| NaOH | To pH > 3 |
| Water (WFI) | To 100% |

In some embodiments, the compositions of the present invention are administered ocularly, for example, using the techniques described herein, and/or other techniques (e.g. injection, topical administration, etc.) known to those in the art (See, e.g., Janoria et al., Expert Opin Drug Deliv., 4(4): 371-388 (July 2007); Ghate & Edelhauser, Expert Opin Drug Deliv., 3(2):275-87 (2006); Bourges et al., Adv Drug Deliv Rev., 58(11):1182-202 (2006), Epub 2006 Sep. 22; Gomes Dos Santos et al., Curr Pharm Biotechnol., 6(1):7-15 (2005); herein incorporated by reference in their entireties). The composition may be administered using any method known to those of ordinary skill in the art. Non-limiting examples include topical, subconjunctival, sub-Tenon's, intravitreal, subretinal, or injection into the anterior chamber of the eye of a subject. Other modes of administration include systemic administration, including intravenous administration as well as oral administration. In certain embodiments, the composition is administered intravitreally.

Certain embodiments relate to a pharmaceutical composition comprising the Compound 1 polypeptide and a pharmaceutically acceptable carrier. Any carrier which can supply a polypeptide without destroying the vector within the carrier is a suitable carrier, and such carriers are well known in the art.

The composition can be formulated and packaged suitably for parenteral, oral, or topical administration. For example, a parenteral formulation would be a sterile, non-pyrogenic product and could consist of a prompt or sustained release liquid preparation, dry powder, emulsion, suspension, or any other standard formulation. An oral formulation of the pharmaceutical composition could be, for example, a liquid solution, such as an effective amount of the composition dissolved in diluents (e.g., water, saline, juice, etc.), suspensions in an appropriate liquid, or suitable emulsions. An oral formulation could also be delivered in tablet form, and could include excipients, colorants, diluents, buffering agents, moistening agents, preservatives, flavoring agents, and pharmacologically compatible excipients. A topical formulation could include compounds to enhance absorption or penetration of the active ingredient through the skin or other affected areas, such as dimethylsulfoxide and related analogs. The pharmaceutical composition could also be delivered topically using a transdermal device, such as a patch, which could include the composition in a suitable solvent system with an adhesive system, such as an acrylic emulsion, and a polyester patch. Sterile compositions could be delivered via eye drops or other topical eye delivery method. Sterile, nonpyrogenic compositions may be delivered intraocularly, anywhere in the eye including, for example, the vitreous cavity, the anterior chamber, etc. Sterile, nonpyrogenic compositions may be delivered intravitrealy as is commonly done with intravitreal injections of Lucentis (ranabizumab), Avastin (bevazizumab), triamcinolone acetonide, antibiotics, etc. Compositions may be delivered periocularly (e.g. to the tissue around the eyeball (globe) but within the bony orbit). Compositions may be delivered via intraocular implant (e.g. gancyclovir implant, fluocinolone implant, etc.). In intraocular implant delivery, devices containing compositions of the present invention are surgically implanted (e.g. within the vitreous cavity), and the drug is released into the eye (e.g. at a predetermined rate). Compositions may be administered using encapsulated cell technology (e.g. by Neurotech) in which genetically modified cells are engineered to produce and secrete composition comprising the Compound 1 polypeptide. Compositions may be delivered via transcleral drug delivery using a device sutured or placed next to the globe that would slowly elute the drug, which would then diffuse into the eye.

Some embodiments relate to compositions, kits, systems, and/or methods to prevent, inhibit, block, and/or reduce photoreceptor, RPE cell or retinal ganglion cell death. Some embodiments relate to inhibition of apoptosis of photoreceptors. Some embodiments relate to inhibition of apoptosis in cells of the retinal pigmented epithelium of the eye. Some embodiments relate to inhibition of apoptosis in cells of the retinal ganglia of the eye. In some embodiments, photoreceptor death and/or apoptosis and/or retinal pigmented epithelium cell apoptosis and/or apoptosis and/or retinal ganglion cell apoptosis and/or death is caused by retinal detachment, age-related macular degeneration, glaucoma, trauma, cancer, tumor, inflammation, uveitis, diabetes, hereditary retinal degeneration, and/or a disease affecting photoreceptor cells, abnormal retinal pigment epithelium or retinal ganglia.

In some embodiments, the present invention enhances photoreceptor, RPE or retinal ganglion cell viability and/or inhibits photoreceptor death (e.g. during retinal detachment and/or is ocular conditions which do not involve retinal detachment.

In some embodiments, the present invention finds utility in enhanced photoreceptor, RPE or retinal ganglion cell viability and/or inhibits photoreceptor, RPE or retinal ganglion cell death in a variety of conditions and/or diseases including, but not limited to macular degeneration (e.g. dry, wet, non-exudative, or exudative/neovascular), ocular tumors, glaucoma, hereditary retinal degenerations (e.g. retinitis pigmentosa, Stargardt's disease, Usher Syndrome, etc.), ocular inflammatory disease (e.g. uveitis), ocular infection (e.g. bacterial, fungal, viral), autoimmune retinitis (e.g. triggered by infection), trauma, diabetic retinopathy, choroidal neovascularization, retinal ischemia, retinal vascular occlusive disease (e.g. branch retinal vein occlusion, central retinal vein occlusion, branch retinal artery occlusion, central retinal artery occlusion, etc.), pathologic myopia, angioid streaks, macular edema (e.g. of any etiology), central serous chorioretinopathy.

Some embodiments relate to administration of a composition to inhibit photoreceptor, RPE or retinal ganglion cell death (e.g. apoptosis). In some embodiments, a composition comprises a pharmaceutical, small molecule, peptide, nucleic acid, molecular complex, etc. In some embodiments, the present invention provides administration of a photoreceptor, RPE or retinal ganglion cell protective polypeptide to inhibit photoreceptor or RPE or retinal ganglion cell apoptosis.

Some embodiments relate to a method of employing a polypeptide to attenuate the activation of one or more members of the TNFR superfamily, desirably Fas or TRAIL in photoreceptors and/or retinas. In some embodiments, such method is employed, for example, to inhibit cell death (e.g., apoptosis) in cells and tissues, and it can be employed in vivo, ex vivo or in vitro. Thus, Compound 1 may be used for attenuating cell death (e.g. retinal cell death) in accordance with such methods. For in vitro application, the Compound 1 may be provided to cells, typically a population of cells (e.g., within a suitable preparation, such as a buffered solution) in an amount and over a time course sufficient to inhibit apoptosis within the cells or to inhibit inflammation. If desired, a controlled population untreated with the inventive polypeptide can be observed to confirm the effect of the inventive polypeptide in reducing the inhibition of cell death or inflammation within a like population of cells.

In some embodiments, provided herein are methods of treating various ocular diseases or conditions (e.g., of the retina) or preventing retinal cell death from resulting from ocular diseases or conditions, including the following: glaucoma, maculopathies/retinal degeneration, such as: macular degeneration, including age-related macular degeneration (AMD), such as non-exudative age-related macular degeneration and exudative age-related macular degeneration; choroidal neovascularization; retinopathy, including diabetic retinopathy, acute and chronic macular neuroretinopathy, central serous chorioretinopathy; and macular edema, including cystoid macular edema, and diabetic macular edema; uveitis/retinitis/choroiditis, such as acute multifocal placoid pigment epitheliopathy, Behcet's disease, birdshot retinochoroidopathy, infectious (syphilis, Lyme Disease, tuberculosis, toxoplasmosis), uveitis, including intermediate uveitis (pars planitis) and anterior uveitis, multifocal choroiditis, multiple evanescent white dot syndrome (MEWDS), ocular sarcoidosis, posterior scleritis, serpignous choroiditis, subretinal fibrosis, uveitis syndrome, and Vogt-Koyanagi-Harada syndrome; vascular diseases/exudative diseases, such as: retinal arterial occlusive disease, central retinal vein occlusion, disseminated intravascular coagulopathy, branch retinal vein occlusion, hypertensive fundus changes, ocular ischemic syndrome, retinal arterial microaneurysms, Coats disease, parafoveal telangiectasis, hemi-retinal vein occlusion, papillophlebitis, central retinal artery occlusion, branch retinal artery occlusion, carotid artery disease (CAD), frosted branch angitis, sickle cell retinopathy and other hemoglobinopathies, angioid streaks, familial exudative vitreoretinopathy, Eales disease, Traumatic/surgical diseases: sympathetic ophthalmia, uveitic retinal disease, retinal detachment, trauma, laser, PDT, photocoagulation, hypoperfusion during surgery, radiation retinopathy, bone marrow transplant retinopathy; proliferative disorders, such as: proliferative vitreal retinopathy and epiretinal membranes, proliferative diabetic retinopathy. Infectious disorders: ocular histoplasmosis, ocular toxocariasis, ocular histoplasmosis syndrome (OHS), endophthalmitis, toxoplasmosis, retinal diseases associated with HIV infection, choroidal disease associated with HIV infection, uveitic disease associated with HIV Infection, viral retinitis, acute retinal necrosis, progressive outer retinal necrosis, fungal retinal diseases, ocular syphilis, ocular tuberculosis, diffuse unilateral subacute neuroretinitis, and myiasis; genetic disorders, such as: retinitis pigmentosa, systemic disorders with associated retinal dystrophies, congenital stationary night blindness, cone dystrophies, Stargardt's disease and fundus flavimaculatus, Best's disease, pattern dystrophy of the retinal pigment epithelium, X-linked retinoschisis, Sorsby's fundus dystrophy, benign concentric maculopathy, Bietti's crystalline dystrophy, pseudoxanthoma elasticum. Retinal tears/holes: retinal detachment, macular hole, giant retinal tear; tumors, such as: retinal disease associated with tumors, congenital hypertrophy of the RPE, posterior uveal melanoma, choroidal hemangioma, choroidal osteoma, choroidal metastasis, combined hamartoma of the retina and retinal pigment epithelium, retinoblastoma, vasoproliferative tumors of the ocular fundus, retinal astrocytoma, intraocular lymphoid tumors; and other diseases and conditions such as: punctate inner choroidopathy, acute posterior multifocal placoid pigmentepitheliopathy, myopic retinal degeneration, acute retinal pigment epithelitis corneal dystrophies or dysplasias and the like.

Certain embodiments provide methods for increasing photoreceptor, RPE or retinal ganglion survival comprising administering a pharmaceutical composition comprising Compound 1, or a pharmaceutically acceptable salt thereof. The pharmaceutical compound may be administered in the form of a composition which is formulated with a pharmaceutically acceptable carrier and optional excipients, adjuvants, etc. in accordance with good pharmaceutical practice. The composition may be in the form of a solid, semi-solid or liquid dosage form: such as powder, solution, elixir, syrup, suspension, cream, drops, paste and spray. As those skilled in the art would recognize, depending on the chosen route of administration (e.g. eye drops, injection, etc.), the composition form is determined. In general, it is preferred to use a sterile, unit dosage form of the inventive inhibitor in order to achieve an easy and accurate administration of the active pharmaceutical compound. In general, the therapeutically effective pharmaceutical compound is present in such a dosage form at a concentration level ranging from about 0.01% to about 1.0% by weight of the total composition: i.e., in an amount sufficient to provide the desired unit dose.

In some embodiments, the pharmaceutical composition may be administered in single or multiple doses. The particular route of administration, product requirements and the dosage regimen will be determined by one of skill in keeping with the condition of the individual to be treated and said individual's response to the treatment. In some embodiments, the composition in a unit dosage form for administration to a subject, comprising a pharmaceutical compound and one or more nontoxic pharmaceutically acceptable carriers, adjuvants or vehicles. The amount of the active ingredient that may be combined with such materials to produce a single dosage form will vary depending upon various factors, as indicated above. A variety of materials can be used as carriers, adjuvants and vehicles in the composition of the invention, as available in the pharmaceutical art. Injectable preparations, such as oleaginous solutions, suspensions or emulsions, may be formulated as known in the art, using suitable dispersing or wetting agents and suspending agents, as needed. The sterile injectable preparation may employ a nontoxic parenterally acceptable diluent or solvent such as sterile nonpyrogenic water or 1,3-butanediol. Among the other acceptable vehicles and solvents that may be employed are 5% dextrose injection, Ringer's injection and isotonic sodium chloride injection (as described in the USP/NF). In addition, sterile, fixed oils may be conventionally employed as solvents or suspending media. For this purpose, any bland fixed oil may be used, including synthetic mono-, di- or triglycerides. Fatty acids such as oleic acid can also be used in the preparation of injectable compositions.

There are several possible routes of drug delivery into the ocular tissues. The route of administration depends on the target tissue. In certain embodiments, the routes of administration may be conventional routes of administrations, such as either topical or systemic. Topical administration, mostly in the form of eye drops may be employed to treat disorders affecting the anterior segment of the eye. Administration may also be via direct injection, e.g., intravitreal injection, which involves injection of a drug solution directly into the vitreous humor (VH) using, e.g., 30 G needle. Other routes of administration, e.g., using drug carriers may also be suitable.

In some embodiments, the composition may be administered ocularly (i.e., to the eye), for example, using the techniques described herein, and/or other techniques (e.g. injection, topical administration, etc.) known to those in the art (See, e.g., Janoria et al. Expert Opinion on Drug Delivery. July 2007, Vol. 4, No. 4, Pages 371-388; Ghate & Edelhauser. Expert Opin Drug Deliv. 2006 March; 3(2):275-87; Bourges et al. Adv Drug Deliv Rev. 2006 Nov. 15; 58(11):1182-202. Epub 2006 Sep. 22; Gomes Dos Santos et al. Curr Pharm Biotechnol. 2005 February; 6(1):7-15; herein incorporated by reference in their entireties.

In some embodiments, the composition may be co-administered with one or more other agents for effective protection of photoreceptors and/or inhibition of apoptosis.

In some embodiments, kits are provided comprising Compound 1, or pharmaceutical preparations thereof. In some embodiments, kits further provide devices, materials, buffers, controls, instructions, containers (e.g., vials, syringes), etc. (e.g., for administration). For example, any of the above mentioned compositions and/or formulations may be packaged. Any of the above mentioned compositions and formulations may be distributed in prefilled syringes. The composition and processing result in a sterile, non-pyrogenic product. The package functions to maintain the sterility of the product.

In further embodiments, provided herein are Fas inhibitors, compositions thereof, pharmaceutical preparations thereof, as well as therapeutic methods.

### Fas Inhibitors

Certain embodiments relate to Fas inhibitors and their use in methods of inhibiting Fas activation and/or signaling leading to preventing, treating, or ameliorating various diseases or conditions. Importantly, by inhibiting Fas activation and/or signaling, inflammation-mediated and/or complement-mediated diseases or conditions may be prevented, treated and/or ameliorated.

As used herein the term “Fas inhibitor” refers to a compound capable of inhibiting or reducing Fas receptor activation and/or signaling either via classical pathways or via indirect pathways. Fas inhibitor may bind to the Fas receptor and directly or indirectly affect the gene and protein expression or activity of molecules downstream of the Fas pathway, to prevent inflammation-mediated and/or complement-mediated diseases or conditions. Fas inhibitors are described in detail below and include any derivatives, fragments, and pharmaceutically acceptable salts of the described Fas inhibitors. As used herein, the term “pharmaceutically acceptable salt” refers to any acid or base of a pharmaceutical agent or an active metabolite or residue thereof. As is known to those of skill in the art, “salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts. Fas inhibitors may also include gene therapy agents. For example, Fas inhibitors may include polynucleotides (e.g., Fas polynucleotide antagonists, such as short interfering RNAs (siRNA) or clustered regularly interspaced short palindromic repeat RNAs (CRISPR-RNA or crRNA, including single guide RNAs (sgRNAs) having a crRNA and tracrRNA sequence, as described in more detail below.

The term “Fas-mediated” means involving or depending on the Fas receptor and/or its activation.

Exemplary Fas inhibitors for use in the described methods are provided below.

In certain embodiments, Fas inhibitors for use in the described methods include any Met and Met-derived peptides and/or fragments. The Met protein has been described previously in U.S. Pat. Pub Nos. US 2007/0184522 and US 2008/0280834, and by Wang et al., Molecular Cell, 9:411-421 (2002) and Zou et al., Nature Medicine, 13(9):1078-1085 (2007), which are incorporated by reference in their entirety. The Met protein, also called c-Met or hepatocyte growth factor receptor (HGF receptor), is encoded by the Met gene. Met is comprised of two major subunits: the α and β subunits. Met and fragments of Met, including the extracellular domain of Met and its α subunit, have been shown to bind to Fas and prevent cells from undergoing apoptosis (Wang et al., Molecular Cell, 9:411-421 (2002)). The Met-Fas interaction is thought to sequester Fas and prevent its trimerization, thereby preventing FasL trimers from binding a trimerized receptor complex. Certain Met-derived peptides, include Met-12, have been shown to have similar effects, leading to Fas inhibition to promote cell survival (Zou et al., Nature Medicine, 13(9):1078-1085 (2007)).

Another example of Fas inhibitor is Met-12 (Met-12 has been previously described in U.S. Pat. No. 8,343,931, which is incorporated herein in its entirety), a derivative, and a pharmaceutically active salt thereof.

A further example of Fas inhibitor includes Compound 1 of Formula 1, which is a C-terminal amide peptide of Met-12, a derivative, and a pharmaceutically active salt thereof:

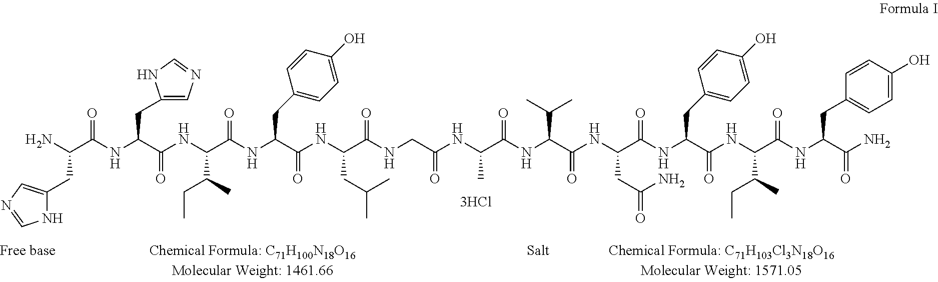 [SMILES: CC[C@H](C)[C@H](NC(=O)[C@H](Cc1ccc(O)cc1)NC(=O)[C@H](CC(N)=O)NC(=O)[C@@H](NC(=O)[C@H](C)NC(=O)CNC(=O)[C@H](CC(C)C)NC(=O)[C@H](Cc1ccc(O)cc1)NC(=O)[C@@H](NC(=O)[C@H](Cc1cnc[nH]1)NC(=O)[C@@H](N)Cc1cnc[nH]1)[C@@H](C)CC)C(C)C)C(=O)N[C@@H](Cc1ccc(O)cc1)C(N)=O]

Compound I/Formula I:
(SEQ ID NO: I)
His-His-Ile-Tyr-Leu-Gly-Ala-Val-Asn-Tyr-Ile-Tyr-
amide

Other examples of Fas inhibitors include derivatives or analogs, and pharmaceutically acceptable salts of Met-12 peptide or Compound 1, including Compounds II-VIII below:

Formula II/Compound 2:

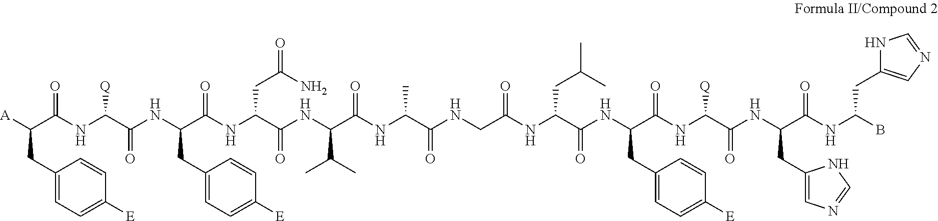

Formula II/Compound 2:
(SEQ ID NO: 2)
H-D-Tyr-D-Ile-D-Tyr-D-Asn-D-Val-D-Ala-Gly-D-Leu-
D-Tyr-D-Ile-D-His-D-His-NH2.

wherein:

A is H—, OH—, NH₂—, G¹(CH₂)ₙ—, R¹CONH—, or R²O—;

B is —H, CH₂OH, CH₂OR², —CHO, —CO₂R², —CONH₂, —CONHR², —CONR³₂, —CONH(CH₂)_{y}NR³₂, —(CH₂)ₙ-G¹, —COCH₂-G¹, —CONHCH₂-G¹, —(CH₂)ₙNH₂, —(CH₂)ₙNHR², —(CH₂)ₙNR³₂, NH-[D]Glu-[D]-His-OH, NH-[D]Glu-[D]-His-NH², -[D]Ala-[D]-His-NH₂, -Gly[D]-His-NH₂, or CONH(CH₂)ₙ-G²;

E, at each occurrence, is independently —H, —OH, OR⁴, SH, SR⁴, or halogen;

G¹, at each occurrence, is independently —H, —C(═O)NH², —C(═O)NHR², —C(═O)NR³₂, C(═O)OR², or —C(═O)R¹;

G² at each occurrence is a heteroalicyclic ring of 4-7 members comprising at least one tertiary amine functionality NR² within the ring, or an alicyclic ring of 3-7 members substituted with NR³₂;

L, at each occurrence, is a multivalent polyethylene glycol derivative with 2-4 termini, each of which may be independently capped with H, R⁵ or another molecule of the peptide of Formula I;

Q, at each occurrence, is independently, [R]-1-methylethyl, [S]-1-methylethyl, 2-propyl, 2-methyl-prop-2-yl, C₃₋₆-cycloalkyl, C₄₋₆-cycloalkenyl, [R]- or [S]-tetrahydrofuran-2-yl, [R]- or [S]-tetrahydrofuran-3-yl, [R]- or [S]-tetrahydrothienyl-2-yl, [R]- or [S]-tetrahydrothienyl-3-yl, [R]- or [S]-tetrahydropyran-2-yl, [R]- or [S]-tetrahydropyran-3-yl, [R]- or [S]-tetrahydropyran-4-yl, [R]- or [S]-tetrahydrothiopyran-2-yl, [R]- or [S]-tetrahydrothiopyran-3-yl, tetrahydrothiopyran-4-yl or [R]- or [S]-1-(R⁵O)ethyl;

R¹, at each occurrence, is independently H, C₁₋₆alkyl, —(CH₂)ₓ(OCH₂CH₂)ₘOR⁵, C₁₋₆ alkoxy or L;

R², at each occurrence, is independently C₁₋₆alkyl, C₂₋₆alkyl substituted with OR⁵ or NR⁵₂, —(CH₂)ₓ(OCH₂CH₂)ₘOR⁵ or L;

R³, at each occurrence, is independently C₁₋₆alkyl, C₂₋₆alkyl substituted with OR⁵ or NR⁵₂, —(CH₂)ₓ(OCH₂CH₂)ₘOR⁵;

or two R³s, taken together with the N atom to which they are attached, may form a monocyclic ring of 4-8 members or a fused, bridged or spiro bicyclic ring of 6-10 members, which can include up to two groups within the ring chosen independently from —O—, —(C═O)—, NR⁶, S, SO, or SO₂;

R⁴, at each occurrence, is independently C₁₋₆alkyl, C₁₋₆acyl, or —OPO₃R⁵₂;

R⁵, at each occurrence, is independently H or C₁₋₆alkyl;

R⁶, at each occurrence, is H, C₁₋₆alkyl, C₂₋₆hydroxyalkyl, C₁₋₆alkoxy-, C₁₋₆alkyl, or C₁₋₆acyl;

m=1-100;

n=0-3;

x=0-6; and

y=2-4, and

wherein at most one of R¹ and R² is L.

Formula III/Compound 3:

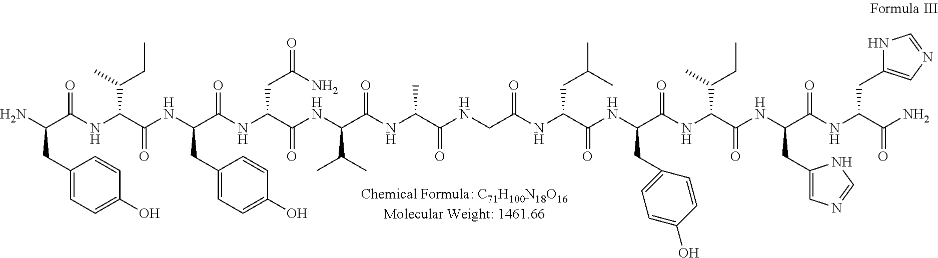 [SMILES: CC[C@@H](C)[C@@H](NC(=O)[C@H](N)Cc1ccc(O)cc1)C(=O)N[C@H](Cc1ccc(O)cc1)C(=O)N[C@H](CC(N)=O)C(=O)N[C@@H](C(=O)N[C@H](C)C(=O)NCC(=O)N[C@H](CC(C)C)C(=O)N[C@H](Cc1ccc(O)cc1)C(=O)N[C@@H](C(=O)N[C@H](Cc1cnc[nH]1)C(=O)N[C@H](Cc1cnc[nH]1)C(N)=O)[C@H](C)CC)C(C)C]

Compound 3/Formula 3:
(SEQ ID NO: 3)
All [D]Tyr-Ile-Tyr-Asn-Val-Ala-Gly-Leu-Tyr-Ile-
His-His-amide

Formula IV/Compound 4:

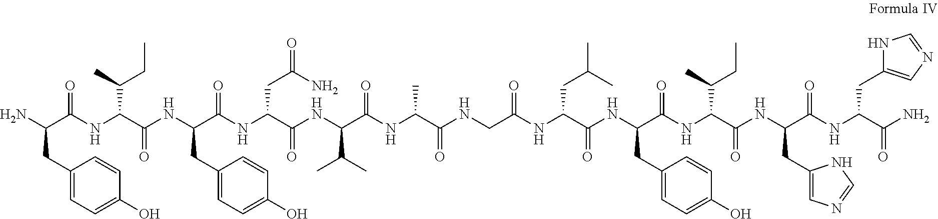 [SMILES: CC[C@H](C)[C@@H](NC(=O)[C@H](N)Cc1ccc(O)cc1)C(=O)N[C@H](Cc1ccc(O)cc1)C(=O)N[C@H](CC(N)=O)C(=O)N[C@@H](C(=O)N[C@H](C)C(=O)NCC(=O)N[C@H](CC(C)C)C(=O)N[C@H](Cc1ccc(O)cc1)C(=O)N[C@@H](C(=O)N[C@H](Cc1cnc[nH]1)C(=O)N[C@H](Cc1cnc[nH]1)C(N)=O)[C@@H](C)CC)C(C)C]

Compound 4/Formula IV:
(SEQ ID NO: 4)
All [D]Tyr-allo-Ile-Tyr-Asn-Val-Ala-Gly-Leu-Tyr-
allo-Ile-His-His-amide

Formula V/Compound 5:

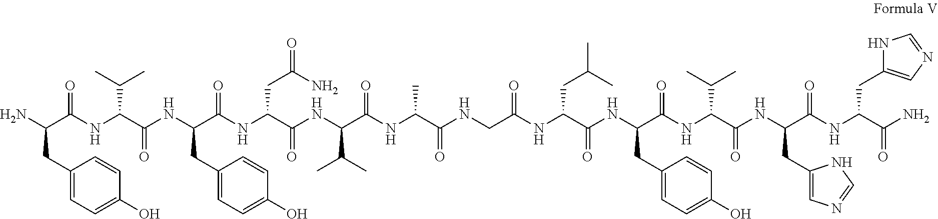 [SMILES: CC(C)C[C@@H](NC(=O)CNC(=O)[C@@H](C)NC(=O)[C@H](NC(=O)[C@@H](CC(N)=O)NC(=O)[C@@H](Cc1ccc(O)cc1)NC(=O)[C@H](NC(=O)[C@H](N)Cc1ccc(O)cc1)C(C)C)C(C)C)C(=O)N[C@H](Cc1ccc(O)cc1)C(=O)N[C@@H](C(=O)N[C@H](Cc1cnc[nH]1)C(=O)N[C@H](Cc1cnc[nH]1)C(N)=O)C(C)C]

Compound 4/Formula IV:
(SEQ ID NO:5 )
All [D]Tyr-Val-Tyr-Asn-Val-Ala-Gly-Leu-Tyr-Val-
His-His-amide

Formula VI/Compound 6:

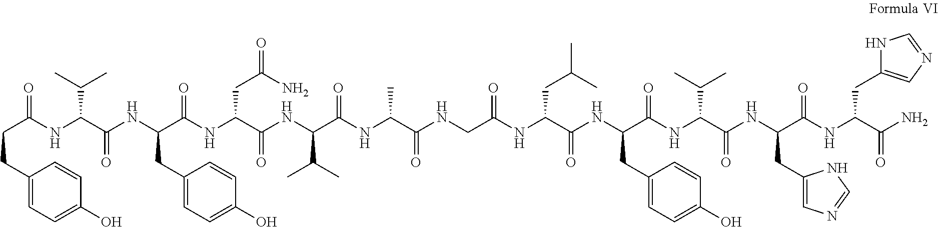

Compound 6/Formula VI:
(SEQ ID NO: 6)
All [D](DesaminoTyr)-Val-Tyr-Asn-Val-Ala-Gly-Leu-
Tyr-Val-His-His-amide

Formula VII/Compound 7:

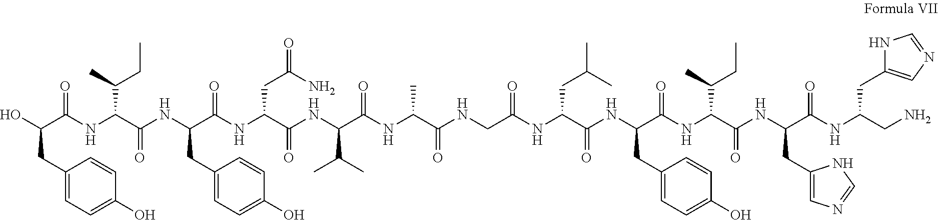 [SMILES: CC[C@H](C)[C@@H](NC(=O)[C@H](O)Cc1ccc(O)cc1)C(=O)N[C@H](Cc1ccc(O)cc1)C(=O)N[C@H](CC(N)=O)C(=O)N[C@@H](C(=O)N[C@H](C)C(=O)NCC(=O)N[C@H](CC(C)C)C(=O)N[C@H](Cc1ccc(O)cc1)C(=O)N[C@@H](C(=O)N[C@H](Cc1cnc[nH]1)C(=O)N[C@@H](CN)Cc1cnc[nH]1)[C@@H](C)CC)C(C)C]

Compound 7/Formula VII:
(SEQ ID NO: 7)
All [D](Hydroxy-desaminoTyr)-allo-Ile-Tyr-Asn-Val-
Ala-Gly-Leu-Tyr-allo-Ile-His-Histamine

Formula VIII/Compound 8:

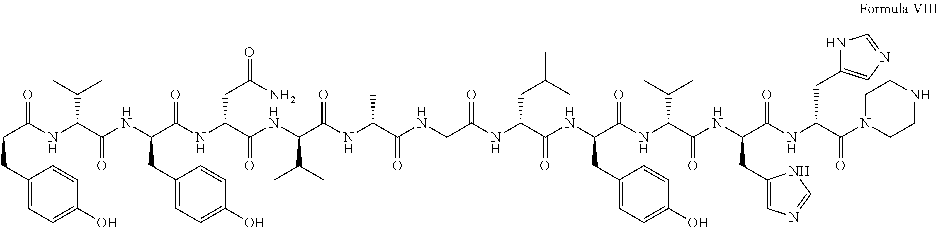

Compound 8/Formula VIII:
(SEQ ID NO: 8)
All [D](DesaminoTyr)-Val-Tyr-Asn-Val-Ala-Gly-Leu-
Tyr-Val-His-His-piperazine amide

In some embodiments, a Fas inhibitor may be a polypeptide comprising any of Compounds I-VIII and can be prepared by methods known to those of ordinary skill in the art. For example, a peptide can be synthesized using solid phase polypeptide synthesis techniques (e.g., Fmoc or tBoc) with D-amino acids. Alternatively, the polypeptide can be synthesized using solution phase techniques, using a wide variety of protected D-amino acids. For example, Compound 2 can be obtained by building the retro-inverso (R-1) Met-12 peptide sequence, (d)Y(d)I(d)Y(d)N(d)V(d)AG(d)L(d)Y(d)I(d)H(d)H (alternatively, “yiynvaglyihh,” using the convention of small letters for d-amino acids and noting that glycine is achiral) onto an amino resin, as is known to those of skill in the art to produce after deprotection and resin cleavage its C-terminal amide, (d)Y(d)I(d)Y(d)N(d)V(d)AG(d)L(d)Y(d)I(d)H(d)H-NH2, Compound 2 (SEQ ID NO:2).

Specifically, although Compound 2 can be obtained conceptually from the c-Met sequence by a normal hydrolysis between residues 59 and 60, and an unnatural breaking of the peptide chain between the peptide nitrogen and the α-carbon of residue 72, rather than at the carbonyl carbon of residue 71, and then reversing the entire sequence whilst exchanging the eleven chiral amino acid residues for their enantiomers, this is not something that could occur naturally, as neither the required bond break between residues 71 and 72, nor the retro-inverso c-Met protein occur in nature. This is not a cleavage, which occurs naturally.

In certain embodiments, analogs or derivatives of Met-12 or C terminal amide thereof can be produced by converting retro-inverso Met-12 into its C-terminal primary amide, to form Compound 2, although it is generally more practical to build up the peptide from an already aminated first amino acid residue, by use of an amino resin, familiar to one of skill in the art.

In certain embodiments, Compounds 1-8 or c-Met, c-Met protein fragments, c-Met polypeptides, and analogs or derivatives of these molecules, such as Met-12, may be linked with various other molecules (e.g. PEG, other active therapeutic molecules, various molecules commonly known as linkers) to optimize delivery, potency, and/or other pharmaceutical properties. These linkers may be covalent and permanent or designed to degrade or be processed over time.

In certain embodiments, c-Met, c-Met protein fragments, c-Met polypeptides, and analogs or derivatives of these molecules may be modified to include amino acids substitutions such as ones known to those skilled in the art including but not limited to substitutions to maintain or modify polarity or size, etc. or substitutions or sequences that contain non-proteinogenic amino acids or various terminal caps or modifications, each or multiple in combination which do not occur naturally.

In certain embodiments, Compounds 1-8 or c-Met protein fragments, c-Met polypeptides, and analogs or derivatives of these molecules, such as Met-12, could be mimicked through petidomimetic strategies by those skilled in the art.

Additional Fas inhibitors include Fas antibody inhibitors, Kp7-6, and viral vector-based gene therapy inhibitors of Fas, including viral vector constructs that lead to the production and/or secretion of Fas inhibiting proteins and viral vector constructs that lead to the production and/or secretion of small peptides like Met12 and analogs, including, e.g., c-MET, c-Met alpha subunit, c-Met alpha subunit modified to prevent binding of HGF.

In certain embodiments, described herein are methods for preventing, treating or ameliorating an inflammation-mediated and/or complement-mediated disease or condition in a subject that involve gene therapy. As used herein, the term “gene therapy” refers to the introduction of extra genetic material in the form of DNA or RNA into the total genetic material in a cell that restores, corrects, or modifies expression of a gene, or for the purpose of expressing a therapeutic polypeptide, e.g., a Fas inhibitor.

Specifically, methods for preventing, treating or ameliorating an inflammation-mediated and/or complement-mediated disease or condition in a subject that comprise administering to the subject a gene therapy encoding the Fas inhibitor in an amount effective to inhibit Fas signaling are described.

Gene therapy uses a gene therapy agent. As used herein, the term “a gene therapy agent” refers to any nucleic acid construct that encodes and results in the expression of a Fas inhibitor, which is capable of transforming a cell in or adjacent to the body lumen. Transformation refers to the process of changing the genotype of a recipient cell by the stable introduction of RNA or DNA by any methodology available to one of ordinary skill in the art. Any gene therapy agent that encodes and results in the expression of a Fas inhibitor may be used.

In order to express a desired polypeptide, e.g., Fas inhibitor, the introduction or delivery of DNA or RNA into cells can be accomplished by multiple methods using a vector (or a vector system), or a carrier. The two major classes of vector systems are recombinant viruses (also referred to as biological nanoparticles or viral vectors), and naked DNA or DNA complexes (non-viral methods, e.g., via a carrier). Both classes of vectors may be used to prepare the gene therapy agents for use in the described methods.

The nucleic acid construct may be an RNA or DNA construct. Examples of types of nucleic acid constructs which may be used as the gene therapy agent include, but are not limited to strands or duplexes of DNA and RNA, DNA and RNA viral vectors and plasmids.

The term “vector” is used herein to refer to a nucleic acid molecule capable transferring or transporting another nucleic acid molecule. The transferred nucleic acid is generally linked to, e.g., inserted into, the vector nucleic acid molecule. A vector may include sequences that direct autonomous replication in a cell, or may include sequences sufficient to allow integration into host cell DNA. Examples of vectors are plasmids (e.g., DNA plasmids or RNA plasmids), autonomously replicating sequences, and transposable elements. Additional exemplary vectors include, without limitation, plasmids, phagemids, cosmids, artificial chromosomes such as yeast artificial chromosome (YAC), bacterial artificial chromosome (BAC), or PI-derived artificial chromosome (PAC), bacteriophages such as lambda phage or M13 phage, and animal viruses. Examples of categories of animal viruses useful as vectors include, without limitation, retrovirus (including lentivirus), adenovirus, adeno-associated virus, herpesvirus (e.g., herpes simplex virus), poxvirus, baculovirus, papillomavirus, and papovavirus (e.g., SV40). Examples of expression vectors are pCIneo vectors (Promega) for expression in mammalian cells; pLenti4N5-DEST™, pLenti6N5-DEST™, and pLenti6.2N5-GW/lacZ (Invitrogen) for lentivirus-mediated gene transfer and expression in mammalian cells. In certain embodiments, useful viral vectors include, e.g., replication defective retroviruses and lentiviruses.

The term “viral vector” may refer either to a virus (e.g., a transfer plasmid that includes virus-derived nucleic acid elements that typically facilitate transfer of the nucleic acid molecule or integration into the genome of a cell; e.g. virus-associated vector), or viral particle capable of transferring a nucleic acid construct into a cell, or to the transferred nucleic acid itself. Constructs may be integrated and packaged into non-replicating, defective viral genomes like Adenovirus, Adeno-associated virus (AAV), or Herpes simplex virus (HSV) or others, including retroviral and lentiviral vectors, for infection or transduction into cells. The vector may or may not be incorporated into the cell's genome. Viral vectors and transfer plasmids contain structural and/or functional genetic elements that are primarily derived from a virus. Exemplary viruses used as vectors include retroviruses, adenoviruses, adeno-associated viruses, lentiviruses, pox viruses, alphaviruses, and herpes viruses. For example, the term “retroviral vector” refers to a viral vector or plasmid containing structural and functional genetic elements, or portions thereof, that are primarily derived from a retrovirus; the term “lentiviral vector” refers to a viral vector or plasmid containing structural and functional genetic elements, or portions thereof, including LTRs that are primarily derived from a lentivirus. The term “hybrid vector” refers to a vector, LTR or other nucleic acid containing both retroviral, e.g., lentiviral, sequences and non-lentiviral viral sequences. In one embodiment, a hybrid vector refers to a vector or transfer plasmid comprising retroviral e.g., lentiviral, sequences for reverse transcription, replication, integration and/or packaging.

The term “construct,” as used herein, refers to a recombinant nucleic acid that has been generated for the purpose of the expression of a specific nucleotide sequence(s), or that is to be used in the construction of other recombinant nucleotide sequences.

The terms “polynucleotide,” or “nucleic acid” are interchangeable and refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase, or by a synthetic reaction. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after synthesis, such as by conjugation with a label. Other types of modifications include, for example, “caps,” substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, .alpha.-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S(“thioate”), P(S)S (“dithioate”), (O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

The “Fas inhibitor polynucleotide” includes polymers of nucleotides of any length, and include DNA and RNA for Fas inhibitors, including fragments thereof.

The term “retrovirus” refers to an RNA virus that reverse transcribes its genomic RNA into a linear double-stranded DNA copy and subsequently covalently integrates its genomic DNA into a host genome. Illustrative retroviruses suitable for use in particular embodiments, include, but are not limited to: Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV) and Rous Sarcoma Virus (RSV) and lentivirus.

The term “lentivirus” refers to a group (or genus) of complex retroviruses. Illustrative lentiviruses include, but are not limited to: HIV (human immunodeficiency virus; including HIV type 1, and HIV type 2); visna-maedi virus (VMV) virus; the caprine arthritis encephalitis virus (CAEV); equine infectious anemia virus (EIAV); feline immunodeficiency virus (FIV); bovine immune deficiency virus (BIV); and simian immunodeficiency virus (SIV).

The terms “lentiviral vector,” “lentiviral expression vector” may be used to refer to lentiviral transfer plasmids and/or infectious lentiviral particles. Where reference is made herein to elements such as cloning sites, promoters, regulatory elements, heterologous nucleic acids, etc., it is to be understood that the sequences of these elements are present in RNA form in the lentiviral particles of the disclosure and are present in DNA form in the DNA plasmids of the disclosure.

As used herein, the term “transfection” refers to the introduction of a nucleic acid into a host cell, such as by contacting the cell with a recombinant AAV virus as described below.

Adeno-associated virus (AAV) is a replication-deficient parvovirus, the single-stranded DNA genome of which is about 4.7 kb in length including 145 nucleotide inverted terminal repeat (ITRs). The ITRs play a role in integration of the AAV DNA into the host cell genome. When AAV infects a host cell, the viral genome integrates into the host's chromosome resulting in latent infection of the cell. In a natural system, a helper virus (for example, adenovirus or herpesvirus) provides genes that allow for production of AAV virus in the infected cell. In the case of adenovirus, genes E1A, E1B, E2A, E4 and VA provide helper functions. Upon infection with a helper virus, the AAV provirus is rescued and amplified, and both AAV and adenovirus are produced. In the instances of recombinant AAV vectors having no Rep and/or Cap genes, the AAV can be non-integrating. In some embodiments, the non-integrating AAV is preferably used to produce the AAV vectors that comprise coding regions of one or more proteins of interest, for example proteins that are more than 500 amino acids in length, are provided. The AAV vector can include a 5′ inverted terminal repeat (ITR) of AAV, a 3′ AAV ITR, a promoter, and a restriction site downstream of the promoter to allow insertion of a polynucleotide encoding one or more proteins of interest, wherein the promoter and the restriction site are located downstream of the 5′ AAV ITR and upstream of the 3′ AAV ITR. In some embodiments, the recombinant AAV vector includes a posttranscriptional regulatory element downstream of the restriction site and upstream of the 3′ AAV ITR. In some embodiments, the AAV vectors disclosed herein can be used as AAV transfer vectors carrying a transgene encoding a protein of interest for producing recombinant AAV viruses that can express the protein of interest in a host cell.

Generation of the viral vector can be accomplished using any suitable genetic engineering techniques well known in the art, including, without limitation, the standard techniques of restriction endonuclease digestion, ligation, transformation, plasmid purification, and DNA sequencing, for example as described in Sambrook et al. (Molecular Cloning: A Laboratory Manual. Cold Spring Harbor Laboratory Press, N.Y. (1989)).

For example, U.S. Pat. No. 9,527,904B2, which is incorporated herein by reference, describes methods for delivery of proteins of interest using adeno-associated virus (AAV) vectors.

In some embodiments, a cell may be transfected with a recombinant AAV virus, e.g. AAV2, including the Fas inhibitor nucleic acid construct to encode and express the Fas inhibitor. For example, AAV vector including Fas inhibitor polynucleotide may be introduced into a target cell, e.g., a Müller or photoreceptor cell. Fas inhibitor may be Met-12, its amide derivative, Compound 1, or any other Fas inhibitor described herein, including derivatives, fragments and salts thereof.

In certain other embodiments, the delivery of a gene(s) or other polynucleotide sequence using viral vectors may be by means of viral infection (“transduction”).

In particular embodiments, host cells transduced with viral vector of the disclosure that expresses one or more polypeptides, are administered to a subject to treat and/or prevent and/or ameliorate inflammation-mediated and/or complement-mediated diseases or conditions described herein

In some embodiments, a cell may be transduced with a retroviral vector, e.g., a lentiviral vector, encoding an engineered Fas inhibitor construct. The transduced cells elicit a stable, long-term, and persistent cell response.

At each end of the provirus are structures called “long terminal repeats” or “LTRs.” The term “long terminal repeat (LTR)” refers to domains of base pairs located at the ends of retroviral DNAs which, in their natural sequence context, are direct repeats and contain U3, R and U5 regions. LTRs generally provide functions fundamental to the expression of retroviral genes (e.g., promotion, initiation and polyadenylation of gene transcripts) and to viral replication. The LTR contains numerous regulatory signals including transcriptional control elements, polyadenylation signals and sequences needed for replication and integration of the viral genome. The viral LTR is divided into three regions called U3, R and U5. The U3 region contains the enhancer and promoter elements. The U5 region is the sequence between the primer binding site and the R region and contains the polyadenylation sequence. The R (repeat) region is flanked by the U3 and U5 regions. The LTR composed of U3, R and U5 regions and appears at both the 5′ and 3′ ends of the viral genome. Adjacent to the 5′ LTR are sequences necessary for reverse transcription of the genome (the tRNA primer binding site) and for efficient packaging of viral RNA into particles (the Psi site).

As used herein, the term “packaging signal” or “packaging sequence” refers to sequences located within the retroviral genome, which are required for insertion of the viral RNA into the viral capsid or particle, see e.g., Clever et al., 1995. J of Virology, Vol. 69, No. 4; pp. 2101-2109. Several retroviral vectors use the minimal packaging signal (also referred to as the psi [′P] sequence) needed for encapsidation of the viral genome. Thus, as used herein, the terms “packaging sequence,” “packaging signal,” “psi” and the symbol “P,” are used in reference to the non-coding sequence required for encapsidation of retroviral RNA strands during viral particle formation.

In various embodiments, vectors may comprise modified 5′ LTR and/or 3′ LTRs. Either or both of the LTR may comprise one or more modifications including, but not limited to, one or more deletions, insertions, or substitutions. Modifications of the 3′ LTR are often made to improve the safety of lentiviral or retroviral systems by rendering viruses replication-defective. As used herein, the term “replication-defective” refers to virus that is not capable of complete, effective replication such that infective virions are not produced (e.g., replication-defective lentiviral progeny). The term “replication-competent” refers to wild-type virus or mutant virus that is capable of replication, such that viral replication of the virus is capable of producing infective virions (e.g., replication-competent lentiviral progeny).

“Self-inactivating” (SIN) vectors refers to replication-defective vectors, e.g., retroviral or lentiviral vectors, in which the right (3′) LTR enhancer-promoter region, known as the U3 region, has been modified (e.g., by deletion or substitution) to prevent viral transcription beyond the first round of viral replication. This is because the right (3 ‘) LTR U3 region is used as a template for the left (5’) LTR U3 region during viral replication and, thus, the viral transcript cannot be made without the U3 enhancer-promoter. In a further embodiment, the 3′LTR is modified such that the U5 region is replaced, for example, with an ideal poly(A) sequence. It should be noted that modifications to the LTRs such as modifications to the 3′LTR, the 5′LTR, or both 3′ and 5′LTRs, are also contemplated herein.

An additional safety enhancement may be provided by replacing the U3 region of the 5′LTR with a heterologous promoter to drive transcription of the viral genome during production of viral particles. Examples of heterologous promoters which may be used include, for example, viral simian virus 40 (SV40) (e.g., early or late), cytomegalovirus (CMV) (e.g., immediate early), Moloney murine leukemia virus (MoMLV), Rous sarcoma virus (RSV), and herpes simplex virus (HSV) (thymidine kinase) promoters. Typical promoters are able to drive high levels of transcription in a Tat-independent manner. This replacement reduces the possibility of recombination to generate replication-competent virus because there is no complete U3 sequence in the virus production system. In certain embodiments, the heterologous promoter has additional advantages in controlling the manner in which the viral genome is transcribed. For example, the heterologous promoter may be inducible, such that transcription of all or part of the viral genome will occur only when the induction factors are present. Induction factors include, but are not limited to, one or more chemical compounds or the physiological conditions such as temperature or pH, in which the host cells are cultured.

In some embodiments, viral vectors may comprise a TAR element. The term “TAR” refers to the “trans-activation response” genetic element located in the R region of lentiviral (e.g., HIV) LTRs. This element interacts with the lentiviral trans-activator (tat) genetic element to enhance viral replication.

The “R region” refers to the region within retroviral LTRs beginning at the start of the capping group (i.e., the start of transcription) and ending immediately prior to the start of the poly A tract. The R region is also defined as being flanked by the U3 and U5 regions. The R region plays a role during reverse transcription in permitting the transfer of nascent DNA from one end of the genome to the other.

The term “FLAP element” refers to a nucleic acid whose sequence includes the central polypurine tract and central termination sequences (cPPT and CTS) of a includes the central polypurine tract and central termination sequences (cPPT and CTS) of a retrovirus, e.g., HIV-1 or HIV-2. Suitable FLAP elements are described in U.S. Pat. No. 6,682,907 and in Zennou, et al., 2000, Cell, 10 1: 173. During HIV-1 reverse transcription, central initiation of the plus-strand DNA at the central polypurine tract (cPPT) and central termination a the central termination sequence (CTS) lead to the formation of a three-stranded DNA structure: the HIV-1 central DNA flap. While not wishing to be bound by any theory, the DNA flap may act as a cis-active determinant of lentiviral genome nuclear import and/or may increase the titer of the virus.

In one embodiment, retroviral or lentiviral transfer vectors comprise one or more export elements. The term “export element” refers to a cis-acting post-transcriptional regulatory element which regulates the transport of an RNA transcript from the nucleus to the cytoplasm of a cell. Examples of RNA export elements include, but are not limited to, the human immunodeficiency virus (HIV) rev response element (RRE) (see e.g., Cullen et al., 1991. J Viral. 65: 1053; and Cullen et al., 1991. Cell 58: 423), and the hepatitis B virus post-transcriptional regulatory element (HPRE). Generally, the RNA export element is placed within the 3′ UTR of a gene, and may be inserted as one or multiple copies.

In other embodiments, expression of heterologous sequences in viral vectors is increased by incorporating post-transcriptional regulatory elements, efficient polyadenylation sites, and optionally, transcription termination signals into the vectors. A variety of posttranscriptional regulatory elements may increase expression of a heterologous nucleic acid at the protein, e.g., woodchuck hepatitis virus post-transcriptional regulatory element (WPRE; Zufferey et al., 1999, J Viral., 73:2886); the post-transcriptional regulatory element present in hepatitis B virus (HPRE) (Huang et al., Mal. Cell. Biol., 5:3864); and the like (Liu et al., 1995, Genes Dev., 9:1766).

Elements directing the efficient termination and polyadenylation of the heterologous nucleic acid transcripts increases heterologous gene expression. Transcription termination signals are generally found downstream of the polyadenylation signal. In particular embodiments, vectors comprise a polyadenylation sequence 3′ of a polynucleotide encoding a polypeptide to be expressed. The term “poly A site” or “poly A sequence” as used herein denotes a DNA sequence which directs both the termination and polyadenylation of the nascent RNA transcript by RNA polymerase II. Polyadenylation sequences may promote mRNA stability by addition of a poly A tail to the 3′ end of the coding sequence and thus, contribute to increased translational efficiency. Efficient polyadenylation of the recombinant transcript is desirable as transcripts lacking a poly A tail are unstable and are rapidly degraded. Illustrative examples of poly A signals that may be used in a vector of the disclosure, includes an ideal poly A sequence (e.g., AATAAA, ATTAAA, AGTAAA), a bovine growth hormone poly A sequence (BGHpA), a rabbit β-globin poly A sequence (rβgpA), or another suitable heterologous or endogenous poly A sequence known in the art.

The “control elements” or “regulatory sequences” present in an expression vector are those non-translated regions of the vector-origin of replication, selection cassettes, promoters, enhancers, translation initiation signals (Shine Dalgarno sequence or Kozak sequence) introns, a polyadenylation sequence, 5′ and 3′ untranslated regions—which interact with host cellular proteins to carry out transcription and translation. Such elements may vary in their strength and specificity. Depending on the vector system and host utilized, any number of suitable transcription and translation elements, including ubiquitous promoters and inducible promoters maybe used.

In particular embodiments, a vector for use in practicing the embodiments described herein including, but not limited to expression vectors and viral vectors, will include exogenous, endogenous, or heterologous control sequences such as promoters and/or enhancers. An “endogenous” control sequence is one which is naturally linked with a given gene in the genome. An “exogenous” control sequence is one which is placed in juxtaposition to a gene by means of genetic manipulation (i.e., molecular biological techniques) such that transcription of that gene is directed by the linked enhancer/promoter. A “heterologous” control sequence is an exogenous sequence that is from a different species than the cell being genetically manipulated.

The term “promoter” as used herein refers to a recognition site of a polynucleotide (DNA or RNA) to which an RNA polymerase binds. An RNA polymerase initiates and transcribes polynucleotides operably linked to the promoter. In particular embodiments, promoters operative in mammalian cells comprise an AT-rich region located approximately 25 to 30 bases upstream from the site where transcription is initiated and/or another sequence found 70 to 80 bases upstream from the start of transcription, a CNCAAT region where N may be any nucleotide.

The term “enhancer” refers to a segment of DNA which contains sequences capable of providing enhanced transcription and in some instances may function independent of their orientation relative to another control sequence. An enhancer may function cooperatively or additively with promoters and/or other enhancer elements. The term “promoter/enhancer” refers to a segment of DNA which contains sequences capable of providing both promoter and enhancer functions.

The term “operably linked,” refers to a juxtaposition wherein the components described are in a relationship permitting them to function in their intended manner. In one embodiment, the term refers to a functional linkage between a nucleic acid expression control sequence (such as a promoter, and/or enhancer) and a second polynucleotide sequence, e.g., a polynucleotide—of interest, wherein the expression control sequence directs transcription of the nucleic acid corresponding to the second sequence.

As used herein, the term “constitutive expression control sequence” refers to a promoter, enhancer, or promoter/enhancer that continually or continuously allows for transcription of an operably linked sequence. A constitutive expression control sequence may be a “ubiquitous” promoter, enhancer, or promoter/enhancer that allows expression in a wide variety of cell and tissue types or a “cell specific,” “cell type specific,” “cell lineage specific,” or “tissue specific” promoter, enhancer, or promoter/enhancer that allows expression in a restricted variety of cell and tissue types, respectively.

Illustrative ubiquitous expression control sequences suitable for use in particular embodiments of the disclosure include, but are not limited to, a cytomegalovirus (CMV) immediate early promoter, a viral simian virus 40 (SV40) (e.g., early or late), a Moloney murine leukemia virus (MoMLV) LTR promoter, a Rous sarcoma virus (RSV) LTR, a herpes simplex virus (HSV) (thymidine kinase) promoter, HS, P7.5, and P11 promoters from vaccinia virus, an elongation factor I-alpha (EFIa) promoter, early growth response I (EGRI), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B 1), heat shock protein 70 kDa (HSP70), β-kinesin (β-KIN), the human ROSA 26 locus Orions et al., Nature Biotechnology 25, 1477-1482 (2007)), a Ubiquitin C promoter (UBC), a phosphoglycerate kinase-I (PGK) promoter, a cytomegalovirus enhancer/chicken β-actin (CAG) promoter, a β-actin promoter and a myeloproliferative sarcoma virus enhancer, negative control region deleted, d1587rev primer-binding site substituted (MND) promoter (Challita et al., J Viral. 69(2):748-55 (1995)).

Additional examples of gene therapy that may be used in the present invention include, but are not limited to those described in U.S. Pat. No. 5,719,131 (cationic amphiphiles); U.S. Pat. No. 5,714,353 (retroviral vectors); U.S. Pat. No. 5,656,465 (non-integrating viruses, e.g., cytoplasmic viruses); U.S. Pat. Nos. 5,583,362; 5,399,346 (primary human cells, e.g., human blood cells used as vehicles for the transfer of human genes encoding therapeutic agents); U.S. Pat. No. 5,334,761 (cationic lipids useful for making lipid aggregates for delivery of macromolecules and other compounds into cells); U.S. Pat. No. 5,283,185 (cationic amphiphiles); U.S. Pat. No. 5,264,618 (cationic lipids); U.S. Pat. No. 5,252,479 (hybrid parvovirus vectors); U.S. Pat. No. 4,394,448 (DNA); each of which are incorporated herein by reference in their entirety.

Transfection of a cell with a gene therapy can be facilitated through the use of a carrier in combination with the gene therapy. Various different carriers have been developed for performing this function. Examples of different carriers which may be used include, but are not limited to, cationic lipids (derivatives of glycerolipids with a positively charged ammonium or sulfonium ion-containing headgroup, e.g., U.S. Pat. No. 5,711,964); cationic amphiphiles (e.g., U.S. Pat. Nos. 5,719,131; 5,650,096); cationic lipids (e.g., U.S. Pat. Nos. 5,527,928; 5,283,185; 5,264,618); and liposomes (e.g., U.S. Pat. Nos. 5,711,964; 5,705,385; 5,631,237), each of the U.S. patents listed above being incorporated herein by reference.

Naked DNA is the simplest method of non-viral transfection and may be used in certain embodiments described herein.

In certain other embodiments, the use of oligonucleotides is also contemplated. The use of synthetic oligonucleotides in gene therapy is to inactivate the genes involved in the disease process. There are several methods by which this is achieved. One strategy uses antisense specific to the target gene to disrupt the transcription of the faulty gene. Another uses small molecules of RNA called siRNA to signal the cell to cleave specific unique sequences in the mRNA transcript of the faulty gene, disrupting translation of the faulty mRNA, and therefore expression of the gene. This is described in more detail below.

A further strategy uses double stranded oligodeoxynucleotides as a decoy for the transcription factors that are required to activate the transcription of the target gene. The transcription factors bind to the decoys instead of the promoter of the faulty gene, which reduces the transcription of the target gene, lowering expression.

To improve the delivery of the new DNA into the cell, the DNA must be protected from damage and its entry into the cell must be facilitated. To this end new molecules, lipoplexes and polyplexes, that have the ability to protect the DNA from undesirable degradation during the transfection process may be used in certain embodiments described herein.

In certain embodiments, plasmid DNA can be covered with lipids in an organized structure like a micelle or a liposome. When the organized structure is complexed with DNA it is called a lipoplex. There are three types of lipids, anionic (negatively charged), neutral, or cationic (positively charged).

Cationic lipids, due to their positive charge, naturally complex with the negatively charged DNA. Also as a result of their charge they interact with the cell membrane, endocytosis of the lipoplex occurs and the DNA is released into the cytoplasm. The cationic lipids also protect against degradation of the DNA by the cell.

Complexes of polymers with DNA are called polyplexes. Most polyplexes consist of cationic polymers and their production is regulated by ionic interactions. One large difference between the methods of action of polyplexes and lipoplexes is that polyplexes cannot release their DNA load into the cytoplasm, so to this end, co-transfection with endosome-lytic agents (to lyse the endosome that is made during endocytosis, the process by which the polyplex enters the cell) such as inactivated adenovirus must occur. However this is not always the case, polymers such as polyethylenimine have their own method of endosome disruption as does chitosan and trimethylchitosan.

Other methods relating to the use of viral vectors in gene therapy, which may be utilized according to certain embodiments of the present disclosure, may be found in, e.g., Kay, M. A. (1997) Chest 111(6 Supp.):138S-142S; Ferry, N. and Heard, J. M. (1998) Hum. Gene Ther. 9:1975-81; Shiratory, Y. et al. (1999) Liver 19:265-74; Oka, K. et al. (2000) Curr. Opin. Lipidol. 11:179-86; Thule, P. M. and Liu, J. M. (2000) Gene Ther. 7:1744-52; Yang, N. S. (1992) Crit. Rev. Biotechnol. 12:335-56; Alt, M. (1995) J Hepatol. 23:746-58; Brody, S. L. and Crystal, R. G. (1994) Ann. NY Acad. Sci. 716:90-101; Strayer, D. S. (1999) Expert Opin. Investig. Drugs 8:2159-2172; Smith-Arica, J. R. and Bartlett, J. S. (2001) Curr. Cardiol. Rep. 3:43-49; and Lee, H. C. et al. (2000) Nature 408:483-8.

In certain embodiments, the use of the RNA interference (RNAi) pathway that is used by cells to regulate the activity of many genes is contemplated. The term “RNA interference” (RNAi), also called post transcriptional gene silencing (PTGS), refers to the biological process in which RNA molecules inhibit gene expression.

In certain embodiments, an RNA interfering agent may be used in the described methods.

An “RNA interfering agent” as used herein, is defined as any agent that interferes with or inhibits expression of a target gene, e.g., a target gene of the invention, by RNA interference (RNAi). Such RNA interfering agents include, but are not limited to, nucleic acid molecules including RNA molecules, which are homologous to the target gene, e.g., a target gene of the invention, or a fragment thereof, short interfering RNA (siRNA), short hairpin RNA (shRNA), and small molecules which interfere with or inhibit expression of a target gene by RNA interference (RNAi).

“RNA interference (RNAi)” is a process whereby the expression or introduction of RNA of a sequence that is identical or highly similar to a target gene results in the sequence specific degradation or PTGS of messenger RNA (mRNA) transcribed from that targeted gene, thereby inhibiting expression of the target gene. RNAi can also be initiated by introducing nucleic acid molecules, e.g., synthetic siRNAs or RNA interfering agents, to inhibit or silence the expression of target genes. As used herein, “inhibition of target gene expression” or “inhibition of marker gene expression” includes any decrease in expression or protein activity or level of the target gene (e.g., a marker gene of the invention) or protein encoded by the target gene, e.g., a marker protein of the invention. The decrease may be of at least 30%, 40%, 50%, 60%, 70%, 80%, 90%, 95% or 99% or more as compared to the expression of a target gene or the activity or level of the protein encoded by a target gene which has not been targeted by an RNA interfering agent.

“Short interfering RNA” (siRNA), also referred to herein as “small interfering RNA” is defined as an agent which functions to inhibit expression of a target gene. These are the effector molecules for inducing RNAi, leading to posttranscriptional gene silencing with RNA-induced silencing complex (RISC). In addition to siRNA, which can be chemically synthesized, various other systems in the form of potential effector molecules for posttranscriptional gene silencing are available, including short hairpin RNAs (shRNAs), long dsRNAs, short temporal RNAs, and micro RNAs (miRNAs). These effector molecules either are processed into siRNA, such as in the case of shRNA, or directly aid gene silencing, as in the case of miRNA. The present invention thus encompasses the use of shRNA as well as any other suitable form of RNA to effect posttranscriptional gene silencing by RNAi. Use of shRNA has the advantage over use of chemically synthesized siRNA in that the suppression of the target gene is typically long-term and stable. An siRNA may be chemically synthesized, may be produced by in vitro by transcription, or may be produced within a host cell from expressed shRNA.

In one embodiment, a siRNA is a small hairpin (also called stem loop) RNA (shRNA). These shRNAs are composed of a short (e.g., 19-25 nucleotides) antisense strand, followed by a 5-9 nucleotide loop, and the complementary sense strand. Alternatively, the sense strand may precede the nucleotide loop structure and the antisense strand may follow. These shRNAs may be contained in plasmids, retroviruses, and lentiviruses.

As used herein, “gene silencing” induced by RNA interference refers to a decrease in the mRNA level in a cell for a target gene by at least about 5%, about 10%, about 20%, about 30%, about 40%, about 50%, about 60%, about 70%, about 80%, about 90%, about 95%, about 99%, about 100% of the mRNA level found in the cell without introduction of RNA interference. In one preferred embodiment, the mRNA levels are decreased by at least about 70%, about 80%, about 90%, about 95%, about 99%, about 100%.

“Gene editing,” or “genome editing” with engineered nucleases is a type of genetic engineering in which DNA is inserted, deleted or replaced in the genome of an organism using engineered nucleases, or “molecular scissors.” These nucleases create site-specific double-strand breaks (DSBs) at desired locations in the genome. The induced double-strand breaks are repaired through nonhomologous end-joining (NHEJ) or homologous recombination (HR), resulting in targeted mutations (‘edits’).

There are three families of engineered nucleases that may be used in certain embodiments described herein: Zinc finger nucleases (ZFNs), Transcription Activator-Like Effector-based Nucleases (TALENs), and CRISPR-Cas system.

“Zinc-finger nucleases” or “ZFNs” are artificial restriction enzymes generated by fusing a zinc finger DNA-binding domain to a DNA-cleavage domain. Zinc finger domains can be engineered to target specific desired DNA sequences and this enables zinc-finger nucleases to target unique sequences within complex genomes. By taking advantage of endogenous DNA repair machinery, these reagents can be used to precisely alter the genomes of higher organisms. Alongside Cas9 and TALEN proteins, ZFN is becoming a prominent tool in the field of genome editing.

A zinc finger nuclease is a site-specific endonuclease designed to bind and cleave DNA at specific positions. There are two protein domains. The first domain is the DNA binding domain, which consists of eukaryotic transcription factors and contain the zinc finger. The second domain is the nuclease domain, which consists of the FokI restriction enzyme and is responsible for the catalytic cleavage of DNA.

The DNA-binding domains of individual ZFNs typically contain between three and six individual zinc finger repeats and can each recognize between 9 and 18 basepairs. If the zinc finger domains are perfectly specific for their intended target site then even a pair of 3-finger ZFNs that recognize a total of 18 basepairs can, in theory, target a single locus in a mammalian genome. The most straightforward method to generate new zinc-finger arrays is to combine smaller zinc-finger “modules” of known specificity. The most common modular assembly process involves combining three separate zinc fingers that can each recognize a 3 basepair DNA sequence to generate a 3-finger array that can recognize a 9 basepair target site.

The non-specific cleavage domain from the type IIs restriction endonuclease FokI is typically used as the cleavage domain in ZFNs. This cleavage domain must dimerize in order to cleave DNA and thus a pair of ZFNs are required to target non-palindromic DNA sites. Standard ZFNs fuse the cleavage domain to the C-terminus of each zinc finger domain. In order to allow the two cleavage domains to dimerize and cleave DNA, the two individual ZFNs must bind opposite strands of DNA with their C-termini a certain distance apart.

In certain embodiments, zinc finger nucleases may be useful to manipulate the genome of a subject, with the Fas receptor gene disrupted by zinc finger nucleases to be save as a potential treatment for many Fas mediated diseases, as described herein. Custom-designed ZFNs that combine the non-specific cleavage domain (N) of FokI endonuclease with zinc-finger proteins (ZFPs) offer a general way to deliver a site-specific DSB to the genome, and stimulate local homologous recombination by several orders of magnitude. Since ZFN-encoding plasmids could be used to transiently express ZFNs to target a DSB to a specific gene locus in human cells, they offer an excellent way for targeted delivery of the therapeutic genes to a pre-selected chromosomal site.

In certain further embodiments, transcription activator-like effector nuclease (TALEN®) technology may be used in connection with the methods described herein, The TALEN® technology leverages artificial restriction enzymes generated by fusing a TAL effector DNA-binding domain to a DNA cleavage domain.

Restriction enzymes are enzymes that cut DNA strands at a specific sequence. Transcription activator-like effectors (TALEs) can be quickly engineered to bind practically any desired DNA sequence. By combining such an engineered TALE with a DNA cleavage domain (which cuts DNA strands), one can engineer restriction enzymes that will specifically cut any desired DNA sequence. When these restriction enzymes are introduced into cells, they can be used for gene editing or for genome editing in situ, a technique known as genome editing with engineered nucleases. Alongside zinc finger nucleases and Cas9 proteins, TALEN is becoming a prominent tool in the field of genome editing.

TAL effectors are proteins that are secreted by Xanthomonas bacteria. The DNA binding domain contains a repeated highly conserved 33-34 amino acid sequence with divergent 12th and 13th amino acids. These two positions, referred to as the Repeat Variable Diresidue (RVD), are highly variable and show a strong correlation with specific nucleotide recognition. This relationship between amino acid sequence and DNA recognition has allowed for the engineering of specific DNA-binding domains by selecting a combination of repeat segments containing the appropriate RVDs.

The non-specific DNA cleavage domain from the end of the FokI endonuclease can be used to construct hybrid nucleases that are active in many different cell types. The FokI domain functions as a dimer, requiring two constructs with unique DNA binding domains for sites in the target genome with proper orientation and spacing. Both the number of amino acid residues between the TALE DNA binding domain and the FokI cleavage domain and the number of bases between the two individual TALEN binding sites appear to be important parameters for achieving high levels of activity.

The simple relationship between amino acid sequence and DNA recognition of the TALE binding domain allows for the efficient engineering of proteins. Once the TALEN constructs have been assembled, they are inserted into plasmids; the target cells are then transfected with the plasmids, and the gene products are expressed and enter the nucleus to access the genome. Alternatively, TALEN constructs can be delivered to the cells as mRNAs, which removes the possibility of genomic integration of the TALEN-expressing protein. Using an mRNA vector can also dramatically increase the level of homology directed repair (HDR) and the success of introgression during gene editing.

TALEN® technology can be used to edit genomes by inducing double-strand breaks (DSB), which cells respond to with repair mechanisms. Non-homologous end joining (NHEJ) reconnects DNA from either side of a double-strand break where there is very little or no sequence overlap for annealing. This repair mechanism induces errors in the genome via insertion or deletion, or chromosomal rearrangement; any such errors may render the gene products coded at that location non-functional. Because this activity can vary depending on the species, cell type, target gene, and nuclease used, it should be monitored when designing new systems. Alternatively, DNA can be introduced into a genome through NHEJ in the presence of exogenous double-stranded DNA fragments. Homology directed repair can also introduce foreign DNA at the DSB as the transfected double-stranded sequences are used as templates for the repair enzymes.

In certain embodiments, the TALEN® technology may be used to correct the genetic errors that underlie disease, such as inflammation-mediated and/or component mediated disease or condition. In theory, the genome-wide specificity of engineered TALEN fusions allows for correction of errors at individual genetic loci via homology-directed repair from a correct exogenous template.

In certain embodiments, the TALEN® technology may be combined with other genome engineering tools, such as meganucleases. The DNA binding region of a TAL effector can be combined with the cleavage domain of a meganuclease to create a hybrid architecture combining the ease of engineering and highly specific DNA binding activity of a TAL effector with the low site frequency and specificity of a meganuclease.

In certain further embodiments, Clustered regularly-interspaced short palindromic repeats (CRISPR) may be used in the methods of treatment of inflammation-mediated and/or component mediated diseases or conditions as described herein.

CRISPR are segments of prokaryotic DNA containing short repetitions of base sequences. CRISPR may be used to edit genomes with unprecedented precision, efficiency, and flexibility.

The CRISPR/Cas system is a prokaryotic immune system that confers resistance to foreign genetic elements such as plasmids and phages, and provides a form of acquired immunity. CRISPR spacers recognize and cut these exogenous genetic elements in a manner analogous to RNA interference in eukaryotic organisms. A set of genes was found to be associated with CRISPR repeats, and was named the cas, or CRISPR-associated, genes. The cas genes encode putative nuclease or helicase proteins, which are enzymes that can cut or unwind DNA. The Cas genes are always located near the CRISPR sequences. There are a number Cas enzymes, but the best known is called Cas9, which comes from Streptococcus pyogenes. By delivering the Cas9 protein and appropriate guide RNAs into a cell, the organism's genome can be cut at any desired location.

Like RNAi, CRISPR interference (CRISPRi) turns off genes in a reversible fashion by targeting, but not cutting a site. The targeted site is methylated so the gene is epigenetically modified. This modification inhibits transcription. Cas9 is an effective way of targeting and silencing specific genes at the DNA level. For instance, CRISPR may be applied to cells to introduce targeted mutations in genes relevant to a specific disease or condition.

Transfection of a cell with a gene therapy agent can be facilitated through the use of a carrier in combination with the gene therapy agent. Various different carriers have been developed for performing this function. Examples of different carriers which may be used include, but are not limited to, cationic lipids (derivatives of glycerolipids with a positively charged ammonium or sulfonium ion-containing headgroup; e.g., U.S. Pat. No. 5,711,964); cationic amphiphiles (e.g., U.S. Pat. Nos. 5,719,131; 5,650,096); cationic lipids (e.g., U.S. Pat. Nos. 5,527,928; 5,283,185; 5,264,618); and liposomes (e.g., U.S. Pat. Nos. 5,711,964; 5,705,385; 5,631,237), each of the U.S. patents listed above being incorporated herein by reference.

### Compositions

Certain embodiments relate to compositions that include the described Fas inhibitor(s), a derivative, fragment, a pharmaceutically acceptable salt thereof, or a gene therapy encoding the described Fas inhibitor in an amount effective to inhibit Fas signaling.

The composition may be a “pharmaceutical composition,” a “pharmaceutical preparation,” or a “pharmaceutical formulation.”

As used herein, the term “pharmaceutical composition” refers to the combination of one or more pharmaceutical agents (e.g., Fas inhibitor) with one or more carriers, inert or active, making the composition especially suitable for diagnostic or therapeutic use in vitro, in vivo or ex vivo. A pharmaceutical composition comprises the physical entity that is administered to a subject, and may take the form of a solid, semi-solid or liquid dosage form, such as tablet, capsule, orally-disintegrating tablet, pill, powder, suppository, solution, elixir, syrup, suspension, cream, lozenge, paste, spray, etc. A pharmaceutical composition may comprise a single pharmaceutical formulation (e.g., extended release, immediate release, delayed release, nanoparticulate, etc.) or multiple formulations (e.g., immediate release and delayed release, nanoparticulate and non-nanoparticulate, etc.).

As used herein, the terms “pharmaceutical preparation” or “pharmaceutical formulation” refer to at least one, but may be two, three or more, pharmaceutical agent(s) (e.g., Fas inhibitor, e.g., Met, Met-12 or Compound 1) in combination with one or more additional components that assist in rendering the pharmaceutical agent(s) suitable for achieving the desired effect upon administration to a subject. The pharmaceutical formulation may include one or more additives, for example pharmaceutically acceptable excipients, carriers, penetration enhancers, coatings, stabilizers, buffers, acids, bases, or other materials physically associated with the pharmaceutical agent to enhance the administration, release (e.g., timing of release), deliverability, bioavailability, effectiveness, etc. of the dosage form. The formulation may be, for example, a liquid, a suspension, a solid, a nanoparticle, emulsion, micelle, ointment, gel, emulsion, coating, etc. A pharmaceutical formulation may contain a single pharmaceutical agent (e.g., Met, Met-12 or Compound 1) or multiple pharmaceutical agents. A pharmaceutical composition may contain a single pharmaceutical formulation or multiple pharmaceutical formulations. In some embodiments, a pharmaceutical agent (e.g., Met, Met-12 or Compound 1) is formulated for a particular mode of administration (e.g., ocular administration (e.g., intravitreal administration, etc.), etc.). A pharmaceutical formulation is sterile, non-pyrogenic and non-toxic to the subject. The terms “pharmaceutical composition” and “pharmaceutical formulation” may be used interchangeably.

Certain embodiments, relate to compositions that include the described Fas inhibitor, a derivative, or a pharmaceutically acceptable salt thereof; and a pharmaceutically acceptable additive. The additive may be selected from carriers, excipients, disintegrators or disintegrating aids, binders, lubricants, coating agents, pigments, diluents, bases, dissolving agents or solubilizers, isotonic agents, pH regulators, stabilizers, propellants, adhesives, and other additives known in the art.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. Pharmaceutically acceptable carriers include carbohydrates such as trehalose, mannitol, xylitol, sucrose, lactose, and sorbitol. Other ingredients for use in formulations may include DPPC, DOPE, DSPC and DOPC. Natural or synthetic surfactants may be used. PEG may be used (even apart from its use in derivatizing the protein or analog). Dextrans, such as cyclodextran, may be used. Bile salts and other related enhancers may be used. Cellulose and cellulose derivatives may be used. Amino acids may be used, such as use in a buffer formulation.

For further examples of carriers, stabilizers and adjuvants see, e.g., Martin, Remington's Pharmaceutical Sciences, 15th Ed., Mack Publ. Co., Easton, Pa. [1975]; herein incorporated by reference in its entirety.

Also, the use of liposomes, microcapsules or microspheres, inclusion complexes, or other types of carriers known in the art, is contemplated.

In certain embodiments, the composition may include at least one non-ionic surfactant. Examples of non-ionic surfactants include Polysorbate 80, Polysorbate 20, Poloxamer 407, and Tyloxapol.

The composition may be in any form suitable for administration to a subject, e.g., solution, pill, ointment, suspension, eye drops, gel, cream, foam, spray, liniment, and powder. As used herein, the term “administration” refers to the act of giving a drug, prodrug, or other agent, or therapeutic treatment (e.g., Fas inhibitor and/or compositions thereof described herein) to a subject (e.g., a subject or in vivo, in vitro, or ex vivo cells, tissues, and organs). Exemplary routes of administration to the human body can be through the eyes (ophthalmic), mouth (oral), skin (transdermal), nose (nasal), lungs (inhalant), oral mucosa (buccal), ear, rectal, by injection (e.g., intravenously, subcutaneously, intratumorally, intraperitoneally, intravitreally, periocularlly, etc.) and the like. Implantable sustained release forms/formulations are also contemplated.

The compositions and methods described herein are particularly applicable for human subjects at risk for or suffering from inflammation-mediated and/or complement-mediated disease or condition, such as retinal disease (e.g., glaucoma, retinal detachment, AMD (dry and wet), diabetic retinopathy, Uveitis, retinal vein occlusion, retinitis pigmentosa or NAION), immunological disease, cancer, amyloid disease (e.g., Alzheimer's disease, type-2 diabetes, Huntington's disease, ALS, or Parkinson's disease), autoimmune disease (e.g., allergy, lupus, or rheumatoid arthritis), an injury caused by ischemia or reperfusion (e.g., stroke), neurodegeneration, and diseases of the central nervous system. The etiology of the disease or condition, itself, may or may not be Fas-mediated, but Fas-mediated signaling through one or more signaling pathways accelerates or amplifies disease symptoms and/or severity.

The compositions for topical use could be in any form deemed suitable by the person skilled in the art to be applied directly on the ocular surface, like e.g., solution, ointment, suspension, eye drops, gel, cream, foam, spray, liniment, powder.

The Fas inhibitor or a composition thereof may administered daily (once, twice, 3 times, 4 times/day, etc.), every other day, every 3 days, weekly, biweekly, monthly, bimonthly, or tri-monthly, etc.

The described Fas inhibitors or compositions thereof may be administered in an amount effective to inhibit Fas and/or Fas signaling. The term “an amount effective” means an amount of a drug or agent (e.g., Compound 1) or its' formulation effective to facilitate a desired therapeutic effect (e.g., inhibition of Fas signaling) in a particular class of subjects (e.g., infant, child, adolescent, adult). U.S. Food and Drug Administration (FDA) recommended dosages are indicative of a therapeutic dose. For example, in the context of this application, the desired therapeutic effect may be preventing or treating inflammation-mediated and/or complement-mediated disease or condition or limiting the severity of inflammation-mediated and/or complement-mediated disease or condition.

For example, an effective amount may be a daily dose of Fas inhibitor in a range, e.g., from about 1 ng to about 1 mg.

In one embodiment, the composition is in the form of eye drops and the described Fas inhibitor is in a concentration between 0.000001% w/v and 2% w/v.

In certain embodiments, compositions comprise one or more additives, such as carriers, diluents and/or excipients suitable for preparing, e.g., ophthalmic compositions. Suitable for preparing ophthalmic compositions are all carriers, diluents or excipients tolerated by the eye. Examples of excipients that may be used in said compositions are Polysorbate 80, polyethylene glycol (e.g., PEG200, PEG400) dextran and the like.

The compositions may comprise carriers for improving the Fas inhibitor's bioavailability by increasing corneal permeability, like e.g. dimethyl sulfoxide, membrane phospholipids and surfactants.

In certain embodiment, such compositions may also comprise carriers apt to increase bioavailability, stability and tolerability of the active principle. For instance, viscosity-increasing agents such as hyaluronic acid, methylcellulose, polyvinyl alcohol, polyvinyl pyrrolidone, etc. may be used.

To prevent contaminations, the described compositions could comprise one or more preservatives having antimicrobial activity, like e.g. benzalchonium chloride (shortened in BAK).

### Uses and Methods

In certain embodiments, the described Fas inhibitors may be used for preventing, treating or ameliorating an inflammation-mediated and/or complement-mediated disease or condition in a subject.

Examples of diseases or conditions that may be treated with the described Fas inhibitors include, e.g., retinal disease (e.g., glaucoma, retinal detachment, AMD (dry and wet), diabetic retinopathy, Uveitis, retinal vein occlusion, inherited retinal degeneration diseases including retinitis pigmentosa, or NAION), immunological disease, cancer, amyloid disease (e.g., Alzheimer's disease, type-2 diabetes, Huntington's disease, ALS, or Parkinson's disease), traumatic injury (e.g. traumatic brain injury), autoimmune disease (e.g., allergy, lupus, or rheumatoid arthritis), an injury caused by ischemia or reperfusion (e.g., stroke), neurodegeneration, and diseases of the central nervous system (e.g., neuropathies and demyelinating diseases such as multiple sclerosis and inflammatory demyelinating diseases).

Certain embodiments relate to methods of inhibiting Fas signaling to prevent, treat, or ameliorate inflammation-mediated and/or complement-mediated diseases or conditions.

Surprisingly, without being bound by the mechanism of action, it was discovered that the inhibition of Fas/Fas signaling results in at least one of the following: reduction of expression or concentration of at least one Fas-mediated inflammation-related gene or protein; reduction of expression or concentration of at least one Fas-mediated complement-related gene or protein, including complement component 3 (C3) and complement component 1q (C1q); reduction of gene or protein expression or concentration of Caspase 8; reduction of gene or protein expression or concentration of one or more components of the inflammasome, including NLRP3 and NLRP2; reduction of gene or protein expression or concentration of one or more C-X-C motif chemokines, including CXCL2 (MIP-2α) and CXCL10 (IP-10); reduction of gene or protein expression or concentration of one or more C-X3-C motif chemokines, including CX3CL1 (fractalkine); reduction of gene or protein expression or concentration of one or more C-C motif chemokines, including CCL2 (MCP-1), CCL3 (MIP-1α), and CCL4 (MIP-1β); reduction of gene or protein expression or concentration of toll-like receptor 4 (TLR4); reduction of gene or protein expression or concentration of one or more interleukin cytokines, including IL-1β, IL-18, and IL-6; reduction of gene or protein expression or concentration of one or more TNF superfamily cytokines, including TNFα; reduction of Fas-mediated Müller cell activation as indicated by reduced GFAP gene or protein expression or concentration; or increase of expression or concentration or prevent the reduction of expression or concentration of at least one pro-survival gene or protein (e.g., cFLIP). The term “Fas-mediated” means involving or depending on the Fas receptor and/or its activation.

As such, certain embodiments relate to a method for preventing, treating, or ameliorating inflammation-mediated and/or complement-mediated disease or condition in a subject including administering to the subject the described Fas inhibitor or a derivative thereof, or a fragment thereof, or a gene therapy encoding the Fas inhibitor in an amount effective to inhibit Fas and/or Fas signaling, and thereby ameliorate or prevent the disease or condition in the subject, wherein the inhibition of Fas and/or Fas signaling results in at least one (or at least two, or at least three, etc., or all) of the following: reduction of expression or concentration of at least one Fas-mediated inflammation-related gene or protein (e.g., TNFα, IL-1β, IP-10, IL-18, MIP1α, IL-6, GFAP, MIP2, MCP-1, or MIP-1β); reduction of expression or concentration of at least one Fas-mediated complement-related gene or protein (e.g., complement component 3 (C3) and complement component 1q (C1q)); reduction of gene or protein expression or concentration of Caspase 8; reduction of gene or protein expression or concentration of one or more components of the inflammasome (e.g., NLRP3 and NLRP2); reduction of gene or protein expression or concentration of one or more C-X-C motif chemokines (e.g., CXCL2 (MIP-2α) and CXCL10 (IP-10)); reduction of gene or protein expression or concentration of one or more C-X3-C motif chemokines (e.g., CX3CL1 (fractalkine)); reduction of gene or protein expression or concentration of one or more C-C motif chemokines (e.g., CCL2 (MCP-1), CCL3 (MIP-1α), and CCL4 (MIP-1β)); reduction of gene or protein expression or concentration of toll-like receptor 4 (TLR4); reduction of gene or protein expression or concentration of one or more interleukin cytokines (e.g., IL-1β, IL-18, and IL-6); reduction of gene or protein expression or concentration of one or more TNF superfamily cytokines (e.g., TNFα); reduction of Fas-mediated Müller cell activation as indicated by reduced GFAP gene or protein expression or concentration; or increase of expression or concentration or prevent the reduction of expression or concentration of at least one pro-survival gene or protein (e.g., cFLIP). The Fas inhibitor may be selected from the group consisting of: Met protein, derivatives, fragments, pharmaceutically acceptable salts thereof; Met-12, derivatives, fragments, pharmaceutically acceptable salts thereof; SEQ ID NOs: 1-8, derivatives, fragments, pharmaceutically acceptable salts thereof; or gene therapy agents encoding the Fas inhibitor. The subject may have or is at risk of having the inflammation-mediated and/or complement-mediated disease or condition

The inflammation-mediated and/or complement-mediated disease or condition may be a retinal disease, immunological disease, cancer, amyloid disease, an injury caused by ischemia or reperfusion, an injury caused by trauma, neurodegeneration, and diseases of the central nervous system. Examples of the amyloid disease include Alzheimer's disease, type-2 diabetes, Huntington's disease, ALS, or Parkinson's disease. An example of the injury by ischemia or reperfusion is stroke. An example of the injury by trauma is traumatic brain injury. Exemplary autoimmune diseases include allergies, lupus, and rheumatoid arthritis. Exemplary retinal diseases include glaucoma, retinal detachment, AMD (dry and wet), diabetic retinopathy, Uveitis, retinal vein occlusion, inherited retinal degeneration including retinitis pigmentosa, and NAION. Examples of diseases of the central nervous system include neuropathy or a demyelinating disease selected from the group consisting of multiple sclerosis and inflammatory demyelinating diseases.

In the described methods, the Fas inhibitor, its derivative, fragment, the gene therapy product, its corresponding interfering RNA (RNAi), or the pharmaceutically acceptable salt thereof may be administered in a pharmaceutical composition comprising the Fas inhibitor, its derivative, fragment, pharmaceutically acceptable salt, or a gene therapy that encodes the Fas inhibitor; and a pharmaceutically acceptable additive, such as carriers, excipients, disintegrators or disintegrating aids, binders, lubricants, coating agents, pigments, diluents, bases, dissolving agents or solubilizers, isotonic agents, pH regulators, stabilizers, propellants, and adhesives.

In the described methods, the Fas inhibitor, its derivative, or the pharmaceutically acceptable salt thereof may be administered via an injection.

A further embodiment relates to a method for preventing, treating or ameliorating an inflammation-mediated and/or complement-mediated disease or condition in a subject comprising administering to the subject a Fas inhibitor selected from the group consisting of Met protein, derivatives, fragments, pharmaceutically acceptable salts thereof; Met-12, derivatives, fragments, pharmaceutically acceptable salts thereof; SEQ ID NOs: 1-8, derivatives, fragments, pharmaceutically acceptable salts thereof; or a gene therapy agents encoding the Fas inhibitor, in an amount effective to inhibit Fas signaling, and thereby prevent, treat or ameliorate the inflammation-mediated and/or complement-mediated disease or condition in the subject. The subject has or is at risk of having the inflammation-mediated and/or complement-mediated disease or condition. The inflammation-mediated and/or complement-mediated disease or condition may be retinal disease (e.g., glaucoma, retinal detachment, AMD (dry and wet), diabetic retinopathy, Uveitis, retinal vein occlusion, inherited retinal degenerations, including retinitis pigmentosa, or NAION), immunological disease, cancer, amyloid disease (e.g., Alzheimer's disease, type-2 diabetes, Huntington's disease, ALS, or Parkinson's disease), an injury caused by ischemia or reperfusion (e.g., stroke), autoimmune disease (e.g., allergy, lupus, or rheumatoid arthritis), neurodegeneration, and diseases of the central nervous system (e.g., neuropathy or a demyelinating disease selected from the group consisting of multiple sclerosis and inflammatory demyelinating diseases). The Fas inhibitor may be administered in a pharmaceutical composition comprising the Fas inhibitor and a pharmaceutically acceptable additive selected from the group consisting of carriers, excipients, disintegrators or disintegrating aids, binders, lubricants, coating agents, pigments, diluents, bases, dissolving agents or solubilizers, isotonic agents, pH regulators, stabilizers, propellants, and adhesives. The Fas inhibitor may be administered via an injection (e.g., an intravitreal injection, intrathecal, intravenous, or periocular injection).

Yet, another embodiment relates to a method for preserving retinal ganglion cells and axon density, or preventing the loss of ganglion cells and axon density in a patient with glaucoma comprising administering to the subject a Fas inhibitor, a derivative thereof, a fragment thereof, a pharmaceutically acceptable salt thereof, or a gene therapy encoding the Fas inhibitor, wherein the preserving or preventing the loss of retinal ganglion cells and axon density, or preventing the loss thereof is due to at least one (or at least two, or all three) of the following: inhibition of microglial/macrophage activation or recruitment; inhibition of at least one of TNF-α, CCL2/MCP-1 or CCL3/MIP-1α gene or protein expression or concentration; or reduction of IL-1β gene or protein expression or protein maturation, wherein the Fas inhibitor is administered to the subject in an amount effective to inhibit Fas signaling. The Fas inhibitor, a derivative thereof, a fragment thereof, a pharmaceutically acceptable salt thereof, or a gene therapy encoding the Fas inhibitor may be administered in a pharmaceutical composition comprising the Fas inhibitor, a derivative thereof, a fragment thereof, a pharmaceutically acceptable salt thereof, or a gene therapy encoding the Fas inhibitor; and a pharmaceutically acceptable additive. The additive may be selected from the group consisting of carriers, excipients, disintegrators or disintegrating aids, binders, lubricants, coating agents, pigments, diluents, bases, dissolving agents or solubilizers, isotonic agents, pH regulators, stabilizers, propellants and adhesives. The composition may be in a form selected from the group consisting of: solution, pill, ointment, suspension, eye drops, gel, cream, foam, spray, liniment, and powder. The administering may be via an injection, wherein the injection is an intravitreal injection, intrathecal, intravenous or periocular injection. The composition may further comprise at least one non-ionic surfactant selected from the group consisting of Polysorbate 80, Polysorbate 20, Poloxamer 407, and Tyloxapol. The Fas inhibitor or the composition comprising the Fas inhibitor may be administered daily, twice daily, every other day, weekly, biweekly, monthly, bimonthly, or tri-monthly. The Fas inhibitor or the composition comprising Fas inhibitor may be administered in a daily dose of from about 1 ng to about 1 mg. The composition may be in the form of eye drops and the Fas inhibitor is in a concentration between 0.000001% w/v and 2% w/v.

Yet, another embodiment relates to a method of treating a subject having an increase (e.g., at least 5%, or at least 10%, etc.) in the mRNA and/or protein expression level(s) of at least one (or at least two, or at least three, etc., or all) of the following gene and/or protein in the subject's eye, as compared to a control: at least one Fas-mediated inflammation-related gene or protein (e.g. TNFα, IL-1β, IP-10, IL-18, MIP1α, IL-6, GFAP, MIP2, MCP-1, or MIP-1β); at least one Fas-mediated complement-related gene or protein (complement component 3 (C3) or complement component 1q (C1q)); Caspase 8; one or more components of the inflammasome (e.g., NLRP3 or NLRP2); one or more C-X-C motif chemokines (e.g., CXCL2 (MIP-2α) or CXCL10 (IP-10)); one or more C-X3-C motif chemokines (e.g., CX3CL1 (fractalkine)); one or more C-C motif chemokines (CCL2 (MCP-1), CCL3 (MIP-1α), and CCL4 (MIP-1β)); toll-like receptor 4 (TLR4); one or more interleukin cytokines (e.g., IL-1β, IL-18, and IL-6); one or more TNF superfamily cytokines (e.g., TNFα); or GFAP gene or protein expression or concentration, the method comprising administering to the subject a Fas inhibitor. The Fas inhibitor may be any Fas inhibitor described herein. For example, the Fas inhibitor may be selected from the group consisting of: Met protein, derivatives, fragments, pharmaceutically acceptable salts thereof; Met-12, derivatives, fragments, pharmaceutically acceptable salts thereof; SEQ ID NOs: 1-8, derivatives, fragments, pharmaceutically acceptable salts thereof; or a gene therapy agents encoding the Fas inhibitor.

Yet, a further embodiment relates to a method of treating a subject having an increase (e.g., at least a 5%, or at least 10%, etc.) in the mRNA and/or protein expression level(s) of at least one (or at least two, or at least three, etc., or all) of the following gene and/or protein in the subject's serum, plasma, whole blood, or cerebrospinal fluid, as compared to a control: at least one Fas-mediated inflammation-related gene or protein (e.g. TNFα, IL-1β, IP-10, IL-18, MIP1α, IL-6, GFAP, MIP2, MCP-1, or MIP-1β); at least one Fas-mediated complement-related gene or protein (complement component 3 (C3) or complement component 1q (C1q)); Caspase 8; one or more components of the inflammasome (e.g., NLRP3 or NLRP2); one or more C-X-C motif chemokines (e.g., CXCL2 (MIP-2α) or CXCL10 (IP-10)); one or more C-X3-C motif chemokines (e.g., CX3CL1 (fractalkine)); one or more C-C motif chemokines (CCL2 (MCP-1), CCL3 (MIP-1α), and CCL4 (MIP-1β)); toll-like receptor 4 (TLR4); one or more interleukin cytokines (e.g., IL-1β, IL-18, and IL-6); one or more TNF superfamily cytokines (e.g., TNFα); or GFAP gene or protein expression or concentration, the method comprising administering to the subject a Fas inhibitor, the method comprising administering to the subject a Fas inhibitor. The Fas inhibitor may be any Fas inhibitor described herein. For example, the Fas inhibitor may be selected from the group consisting of: Met protein, derivatives, fragments, pharmaceutically acceptable salts thereof; Met-12, derivatives, fragments, pharmaceutically acceptable salts thereof; SEQ ID NOs: 1-8, derivatives, fragments, pharmaceutically acceptable salts thereof; or a gene therapy agents encoding the Fas inhibitor.

In certain embodiments, the described compositions may include a pharmaceutical drug or agent. As used herein, the terms “pharmaceutical drug” or “pharmaceutical agent” refer to a compound, peptide, macromolecule, gene therapy agents, nucleic acids, or other entity that is administered (e.g., within the context of a pharmaceutical composition) to a subject to elicit a desired biological response. A pharmaceutical agent may be a “drug” or any other material (e.g., peptide, polypeptide, nucleic acid), which is biologically active in a human being or other mammal, locally and/or systemically. Examples of drugs are disclosed in the Merck Index and the Physicians Desk Reference, the entire disclosures of which are incorporated by reference herein for all purposes.

Treatment in vivo, i.e., by a method where Fas inhibitor (e.g., Met, Met-12 or Compound 1) is administered to a patient, is expected to result in preventing, treating, or ameliorating an inflammation-mediated and/or complement-mediated disease or condition.

It was surprisingly discovered that expression of inflammation-related genes was significantly reduced in animals treated with Compound 1 as compared to the controls. Also, the gene expression of the complement-related proteins was significantly reduced following the treatment with Compound 1. Even more surprisingly, the expression of cFLIP, generally considered to be pro-survival, was decreased in the control animals, and restored to near-baseline in the Compound 1 treated animals.

These data demonstrate that Fas inhibition by Compound 1 reduces the expression of inflammatory genes following elevated IOP, thereby preventing and/or reducing the inflammatory microenvironment induced by elevated IOP. Additionally, the observation that the expression of complement factors C3 and C1q were significantly elevated with microbead injection and were significantly reduced with Compound 1 treatment, suggests that Fas is upstream of complement signaling.

Taken together, these observations suggest that Fas is upstream of a host of inflammatory mediators, and inhibition of one of these downstream factors may not prevent the overall inflammatory microenvironment as effectively as inhibiting Fas.

In view of this, certain embodiments relate to a method for inhibiting Fas as part of a therapeutic strategy for treatment of inflammation-mediated and/or complement-mediated conditions and/or disorders, including glaucoma.

### EXAMPLES

Experiments were conducted during development of the described embodiments to develop a biologically active pharmaceutical formulation of Compound 1 (e.g., for intravitreal administration). The photoreceptor protective properties of Compound 1 were examined in vitro and in vivo following dosing of peptide solutions in DMSO. Compound 1 has poor aqueous solubility above pH˜3 and high tendency to form gels or precipitates in aqueous environments. From the proportional adjustment of efficacious dose in rats to human according to the intra species vitreous volume a target concentration of 10-20 mg/mL was defined as an initial goal, with lower concentrations (0.5-2.0 mg/mL) becoming more desirable as testing demonstrated the surprisingly superior potency and exposure of the described embodiments. (Examples 1-6).

### Example 1: Compound 1 Preparation and Testing

The Compound 1 peptide (Peptide His-His-Ile-Tyr-Leu-Gly-Ala-Val-Asn-Tyr-Ile-Tyr-NH₂; SEQ ID NO:1) was synthesized on Fmoc-Amide-AMS resin via Fmoc chemistry, by multiple suppliers. Fmoc protected amino acids were purchased from GL Biochem. Reagents for coupling and cleavage were purchased from Aldrich. Solvents were purchased from Fisher Scientific.

The peptide chain was assembled on resin by repetitive removal of the Fmoc protecting group and coupling of protected amino acid. DIC and HOBt were used as coupling reagent, and NMM was used as base; 20% piperidine in DMF was used as de-Fmoc-reagent. Ninhydrin test was performed after each coupling to check the coupling efficiency.

After the last coupling, resin was washed and dried, and peptide was cleaved off resin by treating with cleavage cocktail (TFA/Tis/H₂O/DOTA: 95/3/2/2). Peptide was precipitated from cold ether and collected by filtration, 13 g of crude with purity 46% was obtained (yield: 127%).

For each of two preparative purification runs, around 4.4 g of crude peptide was purified by 2-inch polymer column with TFA buffer (buffer A, 0.1% TFA in water; buffer B, 100% acetonitrile), resultant fractions with purity >85% were further purified by 2-inch C18 column with TFA buffer. Collected fractions with purity >95% were lyophilized to dry, and 3.68 g of material as TFA salt with purity >95% was obtained from 8.8 g of crude. To the 10.5 g of peptide (TFA as counter ion), enough HCl aqueous solution was added to dissolve the peptide. Peptide in HCl aqueous solution was lyophilized to dry. 1.4 g final peptide as HCl salt was obtained with purity 97.0%. HPLC 15% ACN in water 0.1% TFA, Venusil XBP-C18 4.6×250 mm 1.0 mL/min; RT 17.79 min. Mass spectrum APCI MH⁺ 1461.5.

Microanalysis. Found: C, 52.21; H, 6.49; N, 15.42; Cl, 6.73. KF, 3.75%. Calculated for C₇₁H₁₀₀N₁₈O₁₆ 3 HCl. 3.4 H₂O: C, 52.24; H, 6.59; N, 15.45; Cl, 6.52. KF, 3.75%. % Active=89.55%.

Later samples of the peptide were still synthesized as the trifluoroacetate salt, but an anion exchange was carried out with acetate, to give Compound 1 as its triacetate salt.

### Example 2: Compound 1 pH-Solubility Profile

Compound 1 obtained as a trihydrochloride salt, as described in Example 1, and was screened for aqueous solubility at different pHs, by carrying out pH titrations according to the following protocol. In some cases Met-12 was run through an identical experimental procedure to determine its solubility pH profile under the same conditions. Multiple previous experiments had failed to find any conditions where Met-12 could be formulated satisfactorily in a largely aqueous medium at any pH above 2.7.

Compound 1 (10 mg) was dissolved in water (270-900 μL) in a 2 mL clear plastic centrifuge tube with vortexing to give a pH ˜2.4 solution. In all cases the peptide formed a clear solution suggesting at low pH a solubility of at least 40 mg/mL. This solution was then diluted with the appropriate amount of cosolvent or other excipient (sugar, surfactant etc.) to produce a clear acidic solution of 10 mg of Compound 1 in 900 μL of the test solution at room temperature (22-23° C.). Small aliquots of a basic solution (usually sodium hydroxide 1.0 M or 0.1 M, but sometimes other bases when investigating buffers) were added using a microliter syringe. Between additions the solution was mixed by vortexing, and the solution was inspected visually for precipitates of various types, turbidity, as a likely sign of microprecipitates, and viscosity to detect gel formation. pH measurements were taken at all these observation points. Some experiments titrated from the endogenous low pH to pH 10, but later titrations were not carried much above pH 7, or sometimes even lower.

Titration of the aqueous Compound 1 solution with sodium hydroxide suggested a slightly better pH-limited solubility than Met-12, with a clear mobile solution to about pH 3.3, as opposed to pH 3.0 for Met-12. However, when titrations were carried out using five buffering bases, Tris, histidine, sodium citrate, sodium borate and sodium phosphate, in place of sodium hydroxide viscosity and signs of aggregation were generally seen in the pH 2.6-2.9 range. Fibril formation was also seen below pH 3 in one or two cases. From these experiments it appears that Compound 1 has no better an aqueous solubility-pH profile than Met-12.

### Example 3: pH-Dependent Solubility of Compound 1 in Cosolvent Mixtures

The pH-dependent solubility of Compound 1 was examined using cosolvents and additives and compared with Met-12 solubility under the same conditions.

The 70% DMSO experiment was similar to the Met-12 titration with a gel forming around pH 5.5, but in this case, probably because of the inability of the C-terminus to ionize, the gel did not re-dissolve at higher pHs.

70% Propylene glycol (PG) improved the solubility of Compound 1 as compared to Met-12, with no gelling occurring until around pH 4.7, as compared to pH 3.2 for Met-12, and then remaining a gel to pH 10. This titration was repeated with lower amounts of PG (35%, 10%), but neither appeared to improve the solubility profile over water alone.

70% PEG400 and 70% glycerol solutions did not appear to be useful, and neither did the two sugar additives, 10% mannitol or 10% trehalose.

From these experiments it was concluded that propylene glycol may be a useful cosolvent under some limiting circumstances for Compound 1, but not for Met-12.

### Example 4: pH-Dependent Solubility of Compound 1 in Non-Ionic Surfactant Mixtures

Surprisingly, some of the surfactants examined provided significant improvements in the pH-solubility profile of Compound 1 whereas none of the surfactants tested improved the pH-solubility profile of Met-12. Compound 1 in the presence of 10% Tyloxapol remained clear, and of acceptable viscosity, until the pH was above 5.87. With 10% Polysorbate 80, the clear solution did not become appreciably viscous until above pH 6.36. With 10% Polysorbate 20, fibrils were observed at pH 3.2 but with no signs of turbidity or gelling until pH 7.14. The 10% Poloxamer 407 was somewhat ambiguous as to where the onset of insolubility might occur, as a second phase was clearly present in the pH 5-9 range, although the solution appeared to be mobile. This appeared to consist of very large clear globules formed in the solution. This is believed to be an artifact of the high Poloxamer concentration, as 15% Poloxamer solutions gel completely at 27° C., whereas 10% Poloxamer does not gel appreciably at 25° C., but various additives can either raise or lower the sol-gel critical temperature, and the usual viscosity measurements for gelling will not pick up the initial appearance of a separate gel phase efficiently. Therefore, it is believed that there was no loss of solubility of the peptide in the mixture, but the high amount of Poloxamer was forming two Poloxamer phases, a sol phase, and a gel phase. Povidone K30 produced a viscous solution at pH 3.60, which gelled above pH 4.0.

Some of the surfactants were then titrated down with respect to the amount of surfactant added. When Polysorbate 20 was titrated down to 3% and 1% concentrations, fibril formation was seen below pH 2.5, but in both cases other signs of precipitation were not seen until pH 4.14 and 3.76 respectively. Poloxamer 407 at 4% produced a small and apparently not increasing, numbers of fibrils at the initiation of the titration, but no other signs of precipitation until above pH 6.2, and at 2% produced a clear solution until above pH 5.6. At 0.5% fibrils were seen in solution once the titration was begun, but no further signs of precipitation were seen until above pH 4.5.

Compound 1 was clearly superior to Met-12 in most of the surfactants examined, notably Polysorbate 80, Poloxamer 407 and Tyloxapol, with the Polysorbate 20 data being somewhat ambiguous due to the initial fibril formation observed, although it was clear than most of the compound was in solution at pH 3-6, in contrast to Met-12 in the same vehicle. Therefore, Compound 1 solubilities were looked at in cosolvent-surfactant mixtures, starting at high additive concentrations, and then lower concentrations of one or both of the additives, in a sparsely populated matrix experimental design.

### Example 5: Mixed Non-Ionic Surfactant/Cosolvent-Solubility Studies of Compound 1

The combination of 70% PG and 10% Polysorbate 80 resulted in a clear solution which became viscous by pH 3.4, and gelled at pH 5.25, which made it no better than 70% PG, and worse than 10% PS-80.

With 70% PG, 3% PS, the viscosity set in at pH 4.6, but the material was still a gel by pH 5.25.

With 35% PG and 3% PS-80, fibrils appeared in solution as low as pH 2.66, and aggregates were seen in solution at pH 3.48.

35% PG, 10% PS-80 showed no signs of fibrils or aggregates, and appreciable viscosity was seen at pH 4.05, and gelling at pH 5.71 (inferior to 10% PS-80 itself).

10% PG and 10% PS-80 produced a clear solution with low viscosity to pH 4.94, and above pH 5.13 began to precipitate material.

10% PG and 3% PS-80 produced a clear solution to pH 3.16, but some precipitation occurred by pH 3.4.

As stated earlier, solution in 10% Polysorbate 20 appeared to give clear mobile solutions all of the way to pH 7, but even at low pH some fibrils were seen, and these tended to increase in number with increasing pH.

Surprisingly, the combination of 10% PG and 10% Polysorbate 20 resulted in good solubility, with the onset of appreciable viscosity only occurring reproducibly above pH 7, and no visual indications of any precipitation being seen. However, on standing overnight, the solution gelled, and the pH dropped by about 0.2 units. Mild agitation reconverted the gel to a liquid, which could be injected.

3% Polysorbate 20, with 10% PG produced a clear mobile solution to pH 5.3, but dropping the PS-20 to 1% produced fibrils below pH 3.

10% PG was added to the 4%, 2% and 0.5% Poloxamer formulations. With the 4% Poloxamer formulation, there appeared to be a slight improvement, no fibrils seen at low pH, and a clear solution to at least pH 5.36. The 2% Poloxamer 10% PG was equally good with a clear mobile solution to pH 5.74. The 0.5% Poloxamer formulation showed fibrils upon initiation of the titration, and showed some turbidity at pH 3.45, but remained of low viscosity until pH 6.

3%, 1% or no PG was added to 2%, 1% and 0.5% Poloxamer formulations, and stability was measured at pH 4.0, pH 5.5 and pH 7.0, after standing for 3 days at RT, using both visual and filtration (see next Example) assays. Visually, fibrils were observed less frequently in newly made up samples, with them being more likely to be observed with less excipients present, and at higher pH, whereas all of the pH 7.0 samples were turbid initially, and some had obvious precipitation. All of the pH 4.0 samples were initially not turbid, with only the 0.5% PX, 0% PG showing slight turbidity after the hold. With the pH 5.5 samples, initial slight turbidity was seen in all of the 0% PG samples, and the 1% PG 0.5% PX sample, but after the hold only the lowest excipient sample (0.5% PX 0% PG) showed slight turbidity. A couple of these samples became slightly turbid upon agitation. The filtration assay told a rather different story. With the 2% Poloxamer formulations, all three pH 4 formulations had >98% recovery after filtration, and at pH 5.5 recovery was >96%, and at pH 7.0 recoveries were still 86-93%. In 1% PX formulations at pH 4, recovery was 97-98% after filtration, and at pH 5.5 87-93%, but at pH 7 was only 1-13%. In the 0.5% PX formulations recovery after filtration at pH 4 was 73-88%, at pH 5.5 12-43%, and at pH 7 there was no recovery after filtration in any of the three samples.

From these experiments, it can be concluded that relatively low amounts of PG as a cosolvent can be moderately helpful with some surfactants, but high concentrations were detrimental, and the predictivity of these formulation effects is not high. In the PX formulations, amounts of PX present and formulation pH are both more important than PG levels. Furthermore, visual read-outs do not necessarily agree with the more reliable filtration assay, and observation of fibrils especially seemed uncorrelated with the amount of filterable drug present.

### Example 6: Lower Concentration Non-Ionic Surfactant-Solubility Studies of Compound 1

In vitro, and later, in vivo efficacy experiments demonstrated that Compound 1 is in the range of 3->10-fold more potent at blocking FasL (or retinal detachment)-induced apoptosis in photoreceptor cells. These surprising results allow for the projected human dose to be dropped into the 25-200 μg/eye range, which reduced the maximum required concentration of the formulated drug to 2.0 mg/m L. A further set of experiments was carried out to find optimal formulation conditions at that concentration, with some experiments looking at even lower drug concentrations. This work all looked at either Poloxamer 407 or Polysorbate 20 as the surfactant. Both visible turbidity and visual estimation of viscosity are useful screening observations, but as discussed above, it was found that they do not always indicate the presence of aggregated peptide, and much of the later work assessed solubility by measuring the amount of drug present in a sample before and after passing it through an 0.2 micron PVDF membrane or a PALL 25 mm 0.2 μM Ultipor Nylon 6,6 filter. Turbid or highly viscous solutions were generally difficult, or impossible to filter, and when they could be filtered, often gave very low drug recoveries. Surprisingly some mobile, clear, solutions also showed large losses upon filtration, so satisfactory formulations were defined as those which gave >90% drug recovery after filtration. It should be noted that all solubility measurements with compounds like Compound 1, which form fibrils may only measure kinetic solubility. Fibril formation may be very slow under many sets of conditions, and one may be measuring solubility of metastable solutions, where a true thermodynamic solubility, relative to the most stable possible fibril form may take days to years to fully achieve. However, formulations were routinely held for 24-72 hours before filtration, in order to avoid at least rapid precipitation after formulation.

An initial experiment looked at 1 mg/mL solutions of Compound 1 in 3% PS-20/3% PG and 2% PX/2% PG at pH 4. All produced clear solutions, with no loss of API upon filtration. The next experiment looked at 2 mg/mL in 2% PG and 0.1%, 0.25% and 0.5% PX, as well as 0.5% PG and PX, at pH ˜3, pH 4, pH 5.5 and pH 7. All appeared clear and mobile except the pH 7, 0.1% PX sample which was slightly turbid, and apparently mobile, but that produced no recovery when filtered. The pH 5.5 recovery there was only 78%, and at pH 4.0 92%. Higher amounts of PX led to complete recovery at pH 3 and 4.0, 93-97% at pH 5.3, and 88-92% at pH 7.0. The high and low PG 0.5% PX were essentially identical, suggesting PG is not very important in this area of the formulation manifold.

Since, during these experiments quite large pH changes were occasionally seen on prolonged standing, a similar experiment with 2.0 mg/mL Compound 1, and 0.25% PX. 2% PG was run at pH 3, 4, 5.5 and 7, whilst comparing self-buffered material (HCl salt titrated with NaOH) versus 10 mM histidine, acetate and citrate buffers, with analysis by filtration assay. The acetate buffer was at least as good a self-buffered material at all pHs, the histidine buffer was marginally worse, and the citrate buffer was markedly inferior at all three of the higher pHs, with only 75% recovery at pH 4, versus 91% for histidine buffer, 92% unbuffered, and 97% for acetate buffer. Acetate buffer was then standardized on.

To examine the effects of isotonicity agents, pH 4 and pH 5.5 10 mM acetate buffered solutions at 2 mg mL Compound 1, with 0.25% PX were examined with 0.5% and 2% PG, 4.5% mannitol, 2% glycerin and 0.8% aqueous sodium chloride. All gave 98-99% recovery at pH 4.0 and 90-94% recovery at pH 5.5, except for the sodium chloride samples, which had recoveries of 89% and 79% at the two pHs. All were in the 230-310 mOsm/L range except the 0.5% PG which was rather hypotonic. Using 10 mM acetate buffer and 4.5% mannitol as isotonicity agent, 2 mg/mL of Compound 1, and pH 4.0 and 5.5, five surfactant conditions were looked at. They were no surfactant, 0.1% PS-20, 0.1% PS-20 plus 0.25% PX, 0.25% PX, and 0.4% PX. The no surfactant gave 0% filtered at high pH and 23% at pH 4.0, and the 0.4% PX and 0.25% PX/0.1% PS-20 mixture gave complete recovery at pH 4.0 and 95% and 91% respectively at pH 5.5. The 0.25% PX was somewhat inferior with 96% and 91% respectively and the 0.1% PS rather inferior in turn with 90% and 65% recovery at pH 4 and 5.5 respectively.

As the isotonicity experiment had suggested hydrochloride may be poor for solubility, an experiment was made using 2 mg/mL of the triacetate salt of Compound 1. Because of the weak acidity of acetic acid, the inherent pH of this salt at 2 mg/mL in water is 3.4-3.6, but despite that samples were dissolved up in 10 mM acetic acid, 4.5% mannitol, and 0.4%, 0.5%, 0.6%, 0.8% and 1.0% PX or 0.4%, 0.5%, 0.75%, 1.0% and 1.5% PS-20, and the pH was adjusted to 4.0 or 5.5. All samples with unadjusted pH (3.4-3.6) showed >97% recovery after filtration, and >98% at pH 4. At pH 5.5, 0.4 and 0.5% PX had 96% recovery, and higher PX concentrations produced 98-99% recovery, whereas the PS-20 formulations were all 92-94% recovery at that pH. From these experiments, an optimized formulation could contain less PX than a similar PS-20 based formulation, but PS-20 is still acceptable, and may avoid some potential issues of PX, even when given at higher concentrations.

### Example 7: In Vitro Efficacy of Compound 1

Cell Culture.

The 661W photoreceptor cell line was generously provided by Muayyad Al-Ubaidi (Department of Cell Biology, University of Oklahoma Health Sciences Center, Oklahoma City, Okla.). The 661W cell line is a photoreceptor line that has been immortalized by the expression of SV40-T antigen under control of the human interphotoreceptor retinol-binding protein (IRBP) promoter (Al-Ubaidi et al., J Cell Biol., 119(6):1681-1687 (1992), herein incorporated by reference in its entirety). 661W cells express cone photoreceptor markers, including blue and green cone pigments, transducin, and cone arrestin (Tan et al., Invest Ophthalmol Vis Sci., (3):764-768 (2004), herein incorporated by reference in its entirety), and can undergo caspase-mediated cell death (Kanan et al., Invest Ophthalmol Vis Sci., 48(1):40-51 (2007), herein incorporated by reference in its entirety).

The 661W cell line was maintained in Dulbecco's modified Eagle's medium containing 10% fetal bovine serum, 300 mg/L glutamine, 32 mg/L putrescine, 40 μL/L β-mercaptoethanol, 40 μg/L hydrocortisone 2 l-hemisuccinate, and 40 μg/L progesterone. The media also contained penicillin (90 U/mL) and streptomycin (0.09 mg/mL). Cells were grown at 37° C. in a humidified atmosphere of 5% CO2 and 95% air.

Activity Assays.

Caspase 3, caspase 8 and caspase 9 activities were measured with colorimetric tetrapeptide cleavage assay kits, per the manufacturer's instructions (BioVision, Mountain View, Calif.). Total (661W/retinal) protein was extracted as per a previously published protocol (Zacks et al., IOVS, 44(3):1262-1267 (2003), herein incorporated by reference in its entirety). One hundred micrograms of total (661W/retinal) protein was incubated with caspase 3 (DEVD-pNA), caspase 8 (IETD-pNA) or caspase 9 substrates (LEHD-pNA) at 200 μM final concentration for 60 minutes. Absorbance was measured at 405 nm in a microplate reader (Spectra-MAX 190, Molecular Devices, Sunnyvale, Calif.). As a negative control, (661W/retinal) protein was incubated with assay buffer without the tetrapeptide. A second negative control was used in which assay buffer alone was incubated with the tetrapeptide. As a positive control, purified caspase 3, caspase 8, or caspase 9 was incubated with the tetrapeptide alone.

Previous experiments conducted during development of the present invention demonstrated that Fas signaling plays a critical role in caspase 8 activation and photoreceptor apoptosis in vivo.

661W cells were treated with a FasL. Addition of the FasL resulted in cell death. Activity of caspase 8 measured in 661W cell lysates increased with increasing concentration of FasL, peaking with the 500 ng/ml dose. 661W cells were treated with 500 ng/ml FasL and measured activity levels at various time points. Caspase 8 activity was significantly increased at 48 hours in 661W cells exposed to FasL. Caspase 8 activity is reliably increased in a dose-dependent manner by 20-30% in different runs.

The assay system described above was used as an in vitro screening system to find potential inhibitors of the Fas-induced Caspase 8 activation pathway. When Met-12 (trihydrochloride salt) was tested in this 661W cell assay, it showed a dose dependent reduction in FasL-induced caspase 8 activation, maximizing at 10 μM, where, depending on the assay, caspase 8 activation is reduced to within 0-25% of baseline. This activity is very dependent on the formulation in which the Met-12 peptide is delivered. The final formulation for Met-12 was diluted 1000-fold for this assay, and to get to the top of the dose curve, which is normally 100 μM, one uses lesser dilutions. Under these circumstances, maximal dose potency was seen with a neat DMSO solution, which delivered the Met-12 peptide as a clear, mobile, liquid, where the apparent pH is well below 3.0. When aqueous based formulations were tried, even where there was no visual evidence of precipitation or aggregation prior to the material being added to the test wells, the formulations showed considerably less dose potency, with the maximum inhibition not being achieved until the 50-100 μM doses.

This is strongly suggestive that regardless of the final physical form in the test wells, aggregation in the dosing solution leads to species with much less available drug, even in these cellular assays, than do true solutions being diluted into the exact same conditions, where presumably they have the same intrinsic potential solubility. By far the most likely explanation for this is that the preformed aggregates in the non-solutions are kinetically and thermodynamically stable enough not to disaggregate into solution at an optimal rate during the duration of the test, whereas the solutions when diluted into the test wells either do not form aggregates, or more likely form different aggregates, which dissolve up more easily. The principal reason for the aggregates being different would be that the peptide is at minimum somewhat less concentrated form when it is moved from its pH or cosolvent-boosted soluble form to a 99% aqueous milieu at pH 7.4. However, since efficient mixing is unlikely in the test wells, and impossible in the eye, and, as discussed below, solvating protons (low pH) and small molecule solvents, are going to diffuse in water much faster than the hydrophobic and bulky peptides, it is highly probable that these peptides rapidly aggregate in either test wells or vitreous humor immediately after dosing. Thus, although the solution dosing is clearly superior to the suspension/gel dosing, there is no guarantee that it will not still sequester a lot of the peptide as insoluble aggregates, and reduce the effective concentration of the drug in a stochastic fashion when administered.

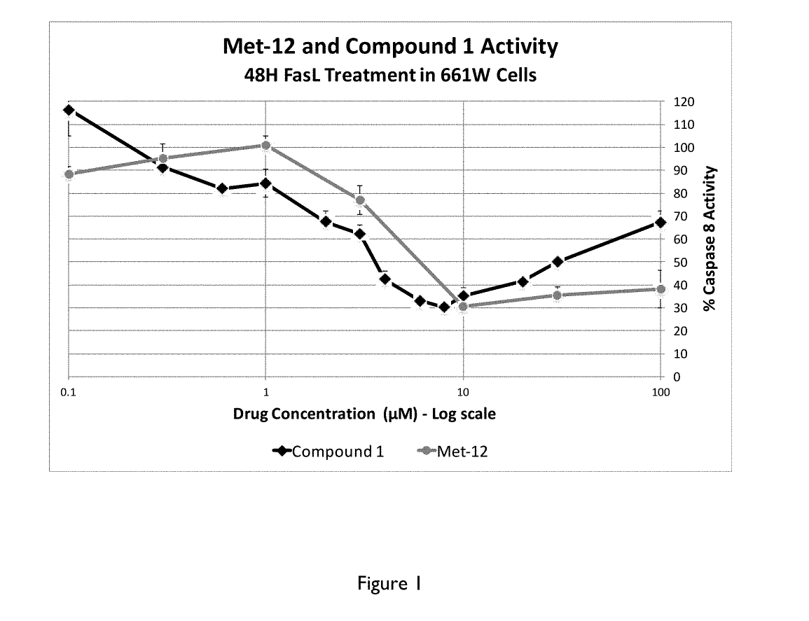

Unexpectedly, as shown in FIG. 1, when Compound 1 trihydrochloride was tested in this assay as an unbuffered solution in DMSO, as compared to Met-12 (also a trihydrochloride in DMSO), it proved to be 10-fold more potent than Met-12 by IC₅₀ determination, and approximately 3-fold more potent measured by the concentration which produced maximal inhibition of FasL-induced caspase 8 activation. The EC₅₀ was 0.4 μM, whereas that for Met-12 itself was 4 μM, and maximal inhibition was seen at 3 μM. However, above 3 μM, Compound 1 showed a U-shaped curve, and above 30 μM, appeared to be almost inactive. In contrast Met-12 dosed in the same manner reaches its (slightly greater) maximum efficacy at 10 μM, and then has only a slight rebound loss of potency out to 100 μM. (See FIG. 1; Caspase 8 activity after 48 hours following treatment with human recombinant FasL after pre-treatment with Met-12 and Compound 1.) 0% is the level of caspase 8 Activity of untreated controls. 100% is set to caspase 8 activity of controls only treated with FasL.

As the mechanism of action of FasL involves a trivalent ligand trimerization of the Fas receptor, it is very difficult to see how univalent Met-12 derivatives could have mixed agonist-antagonist dose curves, and it is believed that the loss of potency is a solubility artifact of the assay. However, the unexpected increase in dose potency for Compound 1 should allow for one to give lower amount of the drug than required for Met-12 itself, which in turn reduces the solubility requirements for the peptide formulation for intravitreal injection.

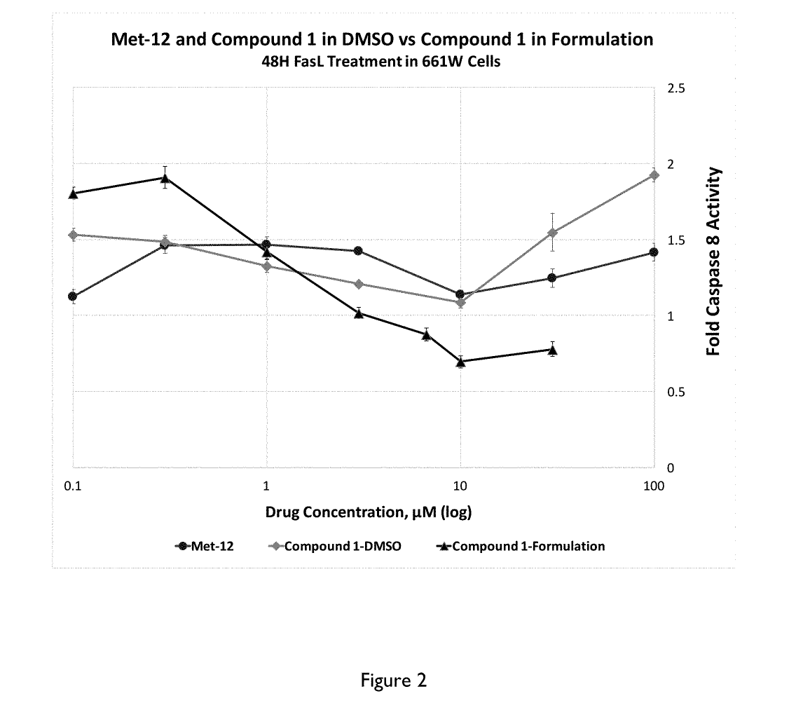

The data shown in FIG. 2 is consistent with the above explanation. It shows once again that Compound 1 trihydrochloride in DMSO (20 mg/mL) is somewhat more dose potent than Met-12, but that it again becomes inactive at higher concentrations. In contrast, when made up at a concentration of 10 mg/mL in a clear, filterable presumably micellar solution of 2% Polysorbate 20 and 2% PG at pH 4, Compound 1 trihydrochloride is both considerably more dose potent, but also more efficacious than Met-12 or Compound 1 in DMSO. Furthermore, there is very little loss of maximum efficacy at the top dose of 30 μM tested in this assay. This surprising result shows the ability to formulate Compound 1 in surfactants can lead to a large boost in drug efficacy.

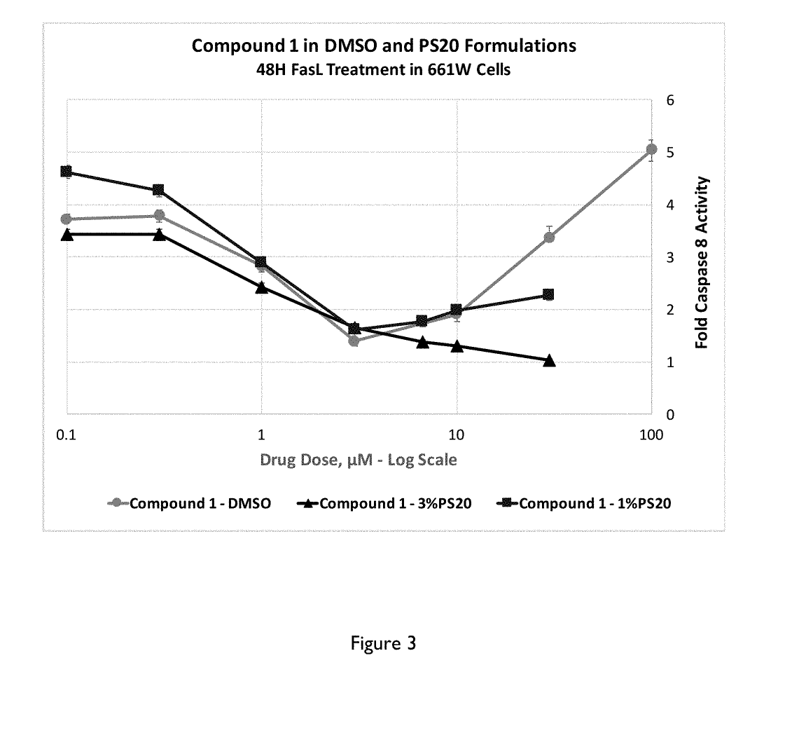

FIG. 3 compares a 20 mg/mL DMSO solution with two Polysorbate formulations of Compound 1 trihydrochloride containing 1% and 3% PS-20 and 3% PG at pH 4 with all at 10 mg/mL concentration. All 3 show similar efficacy in this case at lower concentrations, but the 1% PS-20 only shows a weak loss of activity from its most potent 3 μM concentration, all of the way out to 30 μM, whereas the 3% PS shows continued increase in activity all of the way to 30 μM, the top dose tested.

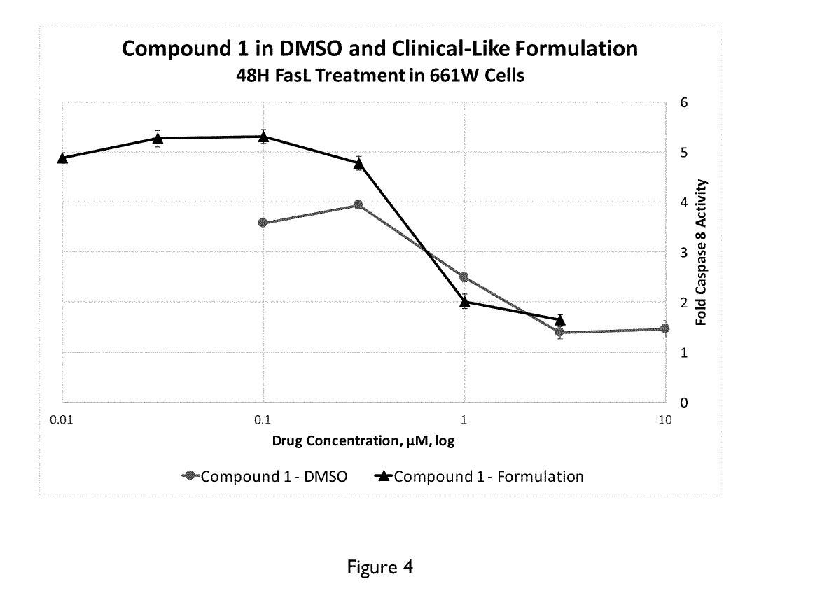

In FIG. 4 an optimized formulations of Compound 1 trihydrochloride, at 2 mg/mL in pH 4 10 mM acetate buffer, 4.5% mannitol and 0.4% PS-20 (dark triangles) is compared to 20 mg/mL Compound 1 trihydrochloride in DMSO (light circles) in 661W cells. The trihydrochloride in DMSO shows the usual U-shaped curve with a maximal effect at 3 μM, and considerable rebound at higher concentrations. The formulation could only be tested to 10 μM, due to its low concentrations, but shows similar efficacy to the DMSO solution.

A likely explanation for greater efficacy seen with the micelle formulations is that the peptide solubility is maintained for a longer period of time by the self-assembling propensity of the surfactant, which is only slowly affected by dilution and dispersion, in sharp contrast to pH gradients or small molecule cosolvents. Thus the micelles and the peptide are more widely dispersed into the aqueous medium, before the peptide is released into the aqueous medium, because the micelles only fall apart slowly, whereas in a cosolvent, or highly acidic solution, the solubilizing factor (hydrogen ions, small molecule), is very rapidly diluted into the aqueous medium, before the peptide has any real chance to disperse beyond the areas into which the injection directly placed it. This will cause the peptide to disperse less efficiently from solution formulations and form more inactive aggregates, than it will from a micellar formulation.

### Example 8: Rabbit Intravitreal Ocular Pharmacokinetic Study with Compound 1

This study was conducted to determine the concentrations of Compound 1 in vitreous humor (VH) and retina tissue following intravitreal injections of male Dutch Belted rabbits. Concentrations were determined in tissues at 24, 72, 168, and 240 hours post-dose of a 50 μL bilateral intravitreal (IVT) dose.

The study design is summarized in Table 1.

TABLE 1
Study design.

| | | | Terminal Timepoints | | |
|---|---|---|---|---|---|
| | | Dose and | (Blood and Ocular | Matrices | Matrices |
| Group | Formulation | Route | Tissues) | Collected | Analyzed |
| 1 (n = 8) | 2 mg/mL Compound 1 | 50 μL/eye | 24, 72, 168, and 240 | Vitreous Humor | Vitreous Humor |
| | triacetate in 4.5% | 0.1 mg/eye | hours postdose | and Retina | and Retina |
| | mannitol, 10 mM acetic | IVT | (n = 2/timepoint) | | |
| | acid, 0.4% poloxamer | | | | |
| | 407, pH 4.5 | | | | |
| 2 (n = 8) | 0.5 mg/mL Compound | 50 μL/eye | 24, 72, 168, and 240 | Vitreous Humor | Vitreous Humor |
| | 1 triacetate in 4.5% | 0.025 mg/eye | hours postdose | and Retina | and Retina |
| | mannitol, 10 mM acetic | IVT | (n = 2/timepoint) | | |
| | acid, 0.4% poloxamer | | | | |
| | 407, pH 4.5 | | | | |
| 3 (n = 8) | 0.5 mg/mL Compound | 50 μL/eye | 24, 72, 168, and 240 | Vitreous Humor | Vitreous Humor |
| | 1 triacetate in 4.5% | 0.025 mg/eye | hours postdose | and Retina | and Retina |
| | mannitol, 10 mM acetic | IVT | (n = 2/timepoint) | | |
| | acid, 0.1% poloxamer | | | | |
| | 407, pH 4.5 | | | | |
| 4 (n = 8) | 1 mg/mL Compound 1 | 50 μL/eye | 24, 72, 168, and 240 | Vitreous Humor | Vitreous Humor |
| | triacetate in 4.5% | 0.05 mg/eye | hours postdose | and Retina | and Retina |
| | mannitol, 10 mM acetic | IVT | (n = 2/timepoint) | | |
| | acid, 0.4% polysorbate | | | | |
| | 20, pH 4.5 | | | | |

Test system included the following:
Species/Strain/Gender: Male Dutch Belted Rabbits
Supplier: Covance Research Products, Inc.
Age Range: 4 to 5 months
Weight at Receipt (Range of Weights): 1.51-1.85 kg
Administration Route: Intravitreal (IVT) injection
Duration of Treatment: Single Dose per eye
Formulation Concentrations: 2.0, 1.0, and 0.5 mg/mL Compound 1 triacetate
Dose Volume: 50 μL per eye

An intravitreal (IVT) injection ocular dose of 50 μL was administered to the globe of each of Dutch Belted rabbit eye.

At the respective time points the rabbits were euthanized by intravenous barbiturate overdose, and eyes were enucleated and snap frozen. Vitreous humor and retina were collected from all animals and analyzed for Compound 1 by LC-MS/MS

Calculations:

Percent Coefficient of Variation:

Used as an estimate of precision. Percent Coefficient of Variation (% CV)=(Standard Deviation/average value)*100

Quadratic Least Squares Analysis:

The standard curve fit was determined using a quadratic equation with 1/x² weighting:

y=ax²+bx+c

where: y=peak area ratio of the calibration standards to internal standard
x=concentration of the calibration standard
a=quadratic coefficient of x²
b=quadratic coefficient of x
c=the constant as the y-intercept of the calibration curve

Quadratic Analyte Concentration:

The concentration of analyte is calculated using the calibration curve parameters calculated above and then solving for the value of x.

Results

Retina and VH concentrations are found in Tables 2 and 3 below, and graphically represented in FIGS. 1 and 2. Pharmacokinetic parameters were calculated where appropriate and are summarized in Tables 4 and 5 below.

TABLE 2
Concentrations of Compound 1 in Dutch Belted retina.

| | | | | | Amount | | | | | | |
|---|---|---|---|---|---|---|---|---|---|---|---|
| | | | | | of | | | | | | |
| | | | | | Diluent | Total | Calculated | Calculated | | | |
| | Sample | Time | | Retina | Added | Volume | Concentration | Concentration | Mean | | |
| Group | ID | Point | Eye | Mass (g) | (μL) | (μL) | (ng/mL) | (ng/g) | (ng/g) | SD | % CV |
| 1 | A | 24 hr | OD | 0.04327 | 173 | 216 | 62.0 | 309 | 596 | 872 | 146 |
| | | | OS | 0.04312 | 172 | 215 | <LLOQ | <LLOQ | | | |
| | B | | OD | 0.06310 | 252 | 315 | 378 | 1890 | | | |
| | | | OS | 0.05021 | 201 | 251 | 36.7 | 183 | | | |
| | A | 72 hr | OD | 0.04984 | 199 | 249 | 25300 | 126000 | 1630 | 1500 | 92.0 |
| | | | OS | 0.04052 | 162 | 203 | 224 | 1120 | | | |
| | B | | OD | 0.04274 | 171 | 214 | 664 | 3320 | | | |
| | | | OS | 0.04199 | 168 | 210 | 89.3 | 447 | | | |
| | A | 168 | OD | 0.04319 | 173 | 216 | 103 | 515 | 354 | 332 | 93.8 |
| | | | OS | 0.04484 | 179 | 224 | <LLOQ | <LLOQ | | | |
| | B | | OD | 0.04184 | 167 | 209 | 147 | 734 | | | |
| | | | OS | 0.03689 | 148 | 185 | 33.5 | 168 | | | |
| | A | 240 | OD | 0.03929 | 157 | 196 | 112 | 559 | 490 | 114 | 23.3 |
| | | | OS | 0.04716 | 189 | 236 | 82.2 | 411 | | | |
| | B | | OD | 0.04788 | 192 | 240 | 122 | 612 | | | |
| | | | OS | 0.05603 | 224 | 280 | 75.2 | 376 | | | |
| 2 | A | 24 hr | OD | 0.03837 | 153 | 191 | 56.8 | 283 | 169 | 121 | 71.6 |
| | | | OS | 0.03881 | 155 | 194 | 35.4 | 177 | | | |
| | B | | OD | 0.03939 | 158 | 197 | 43.2 | 216 | | | |
| | | | OS | 0.04028 | 161 | 201 | <LLOQ | <LLOQ | | | |
| | A | 72 hr | OD | 0.04784 | 191 | 239 | 63.6 | 318 | 223 | 173 | 77.6 |
| | | | OS | 0.04461 | 178 | 223 | <LLOQ | <LLOQ | | | |
| | B | | OD | 0.03850 | 154 | 193 | 36.0 | 180 | | | |
| | | | OS | 0.03466 | 139 | 174 | 78.6 | 395 | | | |
| | A | 168 | OD | 0.04639 | 186 | 232 | 503 | 2520 | 912 | 1070 | 117 |
| | | | OS | 0.04044 | 162 | 202 | 56.7 | 283 | | | |
| | B | | OD | 0.05231 | 209 | 261 | 82.2 | 410 | | | |
| | | | OS | 0.04112 | 164 | 205 | 87.4 | 436 | | | |
| | A | 240 | OD | 0.04847 | 194 | 242 | 66.8 | 334 | 631 | 329 | 52.1 |
| | | | OS | 0.04503 | 180 | 225 | 212 | 1060 | | | |
| | B | | OD | 0.04467 | 179 | 224 | 142 | 712 | | | |
| | | | OS | 0.04772 | 191 | 239 | 83.4 | 418 | | | |
| 3 | A | 24 hr | OD | 0.04893 | 196 | 245 | 24.8 | 124 | 929 | 1450 | 156 |
| | | | OS | 0.04561 | 182 | 228 | 621 | 3100 | | | |
| | B | | OD | 0.05029 | 201 | 251 | 28.0 | 140 | | | |
| | | | OS | 0.04463 | 179 | 224 | 69.8 | 350 | | | |
| | A | 72 hr | OD | 0.04474 | 179 | 224 | 33.0 | 165 | 68.0 | 82.0 | 121 |
| | | | OS | 0.04508 | 180 | 225 | <LLOQ | <LLOQ | | | |
| | B | | OD | 0.04974 | 199 | 249 | 21.3 | 107 | | | |
| | | | OS | 0.04698 | 188 | 235 | <LLOQ | <LLOQ | | | |
| | A | 168 | OD | 0.04188 | 168 | 210 | 832 | 4170 | 1440 | 1890 | 131 |
| | | | OS | 0.03783 | 151 | 189 | <LLOQ | <LLOQ | | | |
| | B | | OD | 0.03577 | 143 | 179 | 74.3 | 372 | | | |
| | | | OS | 0.04405 | 176 | 220 | 247 | 1230 | | | |
| | A | 240 | OD | 0.03215 | 129 | 161 | 104 | 521 | 192 | 248 | 129 |
| | | | OS | 0.04526 | 181 | 226 | <LLOQ | <LLOQ | | | |
| | B | | OD | 0.03895 | 156 | 195 | 49.6 | 248 | | | |
| | | | OS | 0.05110 | 204 | 255 | <LLOQ | <LLOQ | | | |
| 4 | A | 24 hr | OD | 0.04033 | 161 | 201 | 32.1 | 160 | 206 | 237 | 115 |
| | | | OS | 0.04551 | 182 | 228 | 109 | 546 | | | |
| | B | | OD | 0.03931 | 157 | 196 | 23.6 | 118 | | | |
| | | | OS | 0.04407 | 176 | 220 | <LLOQ | <LLOQ | | | |
| | A | 72 hr | OD | 0.04616 | 185 | 231 | <LLOQ | <LLOQ | 95.0 | 111 | 117 |
| | | | OS | 0.04115 | 165 | 206 | 34.4 | 172 | | | |
| | B | | OD | 0.04900 | 196 | 245 | 41.5 | 208 | | | |
| | | | OS | 0.04991 | 200 | 250 | <LLOQ | <LLOQ | | | |
| | A | 168 | OD | 0.03554 | 142 | 178 | <LLOQ | <LLOQ | 51.0 | ISD | ISD |
| | | | OS | 0.03511 | 140 | 175 | 41.0 | 204 | | | |
| | B | | OD | 0.05409 | 216 | 270 | <LLOQ | <LLOQ | | | |
| | | | OS | 0.04863 | 195 | 244 | <LLOQ | <LLOQ | | | |
| | A | 240 | OD | 0.04413 | 177 | 221 | <LLOQ | <LLOQ | 27.3 | ISD | ISD |
| | | | OS | 0.04205 | 168 | 210 | <LLOQ | <LLOQ | | | |
| | B | | OD | 0.04442 | 178 | 222 | <LLOQ | <LLOQ | | | |
| | | | OS | 0.05194 | 208 | 260 | 21.7 | 109 | | | |

LLOQ = 20.0 ng/mL or 100 ng/g
Values < LLOQ use 0 for statistical determination
ISD = Insufficient data for determination
Underlined: Exceeds upper limit of quantitation and outlier, data excluded

TABLE 3
Concentrations of Compound 1 in Dutch Belted vitreous humor.

| | | | | Calculated | Vitreous | Calculated | | | |
|---|---|---|---|---|---|---|---|---|---|
| | Sample | Time | | Concentration | Humor | Concentration | Mean | | |
| Group | ID | Point | Eye | (ng/mL) | Weight (g)* | (μg/eye) | (μg/eye) | SD | % CV |
| 1 | A | 24 hr | OD | 104000 | 1.05875 | 110 | 106 | 8.05 | 7.59 |
| | | | OS | 105000 | 1.09135 | 115 | | | |
| | B | | OD | 78800 | 1.27841 | 101 | | | |
| | | | OS | 75900 | 1.28441 | 97.5 | | | |
| | A | 72 hr | OD | 125000 | 0.71978 | 90.0 | 90.4 | 8.07 | 8.93 |
| | | | OS | 104000 | 0.76824 | 79.9 | | | |
| | B | | OD | 97700 | 0.94602 | 92.4 | | | |
| | | | OS | 106000 | 0.93764 | 99.4 | | | |
| | A | 168 hr | OD | 84800 | 1.16490 | 98.8 | 103 | 5.85 | 5.65 |
| | | | OS | 81500 | 1.18636 | 96.7 | | | |
| | B | | OD | 92600 | 1.17384 | 109 | | | |
| | | | OS | 88500 | 1.20263 | 106 | | | |
| | A | 240 hr | OD | 79400 | 1.17233 | 93.1 | 84.2 | 14.9 | 17.7 |
| | | | OS | 87500 | 1.12218 | 98.2 | | | |
| | B | | OD | 52900 | 1.22366 | 64.7 | | | |
| | | | OS | 65300 | 1.23536 | 80.7 | | | |
| 2 | A | 24 hr | OD | 22000 | 0.84743 | 18.6 | 22.9 | 2.86 | 12.5 |
| | | | OS | 20400 | 1.19418 | 24.4 | | | |
| | B | | OD | 21200 | 1.15341 | 24.5 | | | |
| | | | OS | 21700 | 1.10663 | 24.0 | | | |
| | A | 72 hr | OD | 21300 | 1.09577 | 23.3 | 23.6 | 0.379 | 1.61 |
| | | | OS | 1900 | 1.12046 | 2.13 | | | |
| | B | | OD | 20000 | 1.16980 | 23.4 | | | |
| | | | OS | 20600 | 1.16716 | 24.0 | | | |
| | A | 168 hr | OD | 22300 | 1.12460 | 25.1 | 23.0 | 1.50 | 6.52 |
| | | | OS | 20500 | 1.05482 | 21.6 | | | |
| | B | | OD | 21600 | 1.05841 | 22.9 | | | |
| | | | OS | 18400 | 1.21646 | 22.4 | | | |
| | A | 240 hr | OD | 18000 | 1.17848 | 21.2 | 19.5 | 1.28 | 6.56 |
| | | | OS | 15100 | 1.28698 | 19.4 | | | |
| | B | | OD | 14800 | 1.22182 | 18.1 | | | |
| | | | OS | 16000 | 1.20828 | 19.3 | | | |
| 3 | A | 24 hr | OD | 24500 | 1.21473 | 29.8 | 26.0 | 2.73 | 10.5 |
| | | | OS | 20300 | 1.18169 | 24.0 | | | |
| | B | | OD | 21100 | 1.23315 | 26.0 | | | |
| | | | OS | 19300 | 1.24396 | 24.0 | | | |
| | A | 72 hr | OD | 214 | 1.19559 | 0.256 | 22.8 | 0.814 | 3.57 |
| | | | OS | 18900 | 1.22604 | 23.2 | | | |
| | B | | OD | 21000 | 1.11298 | 23.4 | | | |
| | | | OS | 18900 | 1.15681 | 21.9 | | | |
| | A | 168 hr | OD | 21900 | 1.09436 | 24.0 | 25.5 | 1.98 | 7.76 |
| | | | OS | 26100 | 1.08970 | 28.4 | | | |
| | B | | OD | 20700 | 1.18375 | 24.5 | | | |
| | | | OS | 21300 | 1.18491 | 25.2 | | | |
| | A | 240 hr | OD | 16700 | 1.18857 | 19.8 | 20.4 | 1.13 | 5.54 |
| | | | OS | 18200 | 1.19607 | 21.8 | | | |
| | B | | OD | 16200 | 1.18248 | 19.2 | | | |
| | | | OS | 16700 | 1.23788 | 20.7 | | | |
| 4 | A | 24 hr | OD | 44000 | 1.08887 | 47.9 | 46.8 | 2.55 | 5.45 |
| | | | OS | 40000 | 1.08052 | 43.2 | | | |
| | B | | OD | 38500 | 1.21775 | 46.9 | | | |
| | | | OS | 41600 | 1.17967 | 49.1 | | | |
| | A | 72 hr | OD | 39600 | 1.10963 | 43.9 | 32.5 | 10.2 | 31.4 |
| | | | OS | 33500 | 1.14187 | 38.3 | | | |
| | B | | OD | 23100 | 1.02867 | 23.8 | | | |
| | | | OS | 21700 | 1.09956 | 23.9 | | | |
| | A | 168 hr | OD | 25900 | 1.04704 | 27.1 | 25.6 | 1.12 | 4.38 |
| | | | OS | 21000 | 1.22612 | 25.7 | | | |
| | B | | OD | 19900 | 1.22735 | 24.4 | | | |
| | | | OS | 20000 | 1.26320 | 25.3 | | | |
| | A | 240 hr | OD | 19800 | 1.10090 | 21.8 | 19.4 | ISD | ISD |
| | | | OS | 158 | 1.12301 | 0.177 | | | |
| | B | | OD | 12500 | 1.35282 | 16.9 | | | |
| | | | OS | 177 | 1.38049 | 0.244 | | | |

LLOQ = 50.0 ng/mL
*assume density of 1.00 g/mL
Underlined: Suspected outlier, data not included in statistics
ISD = Insufficient data for determination
Italics: Result exceeds upper limit of quantitation, estimated data.

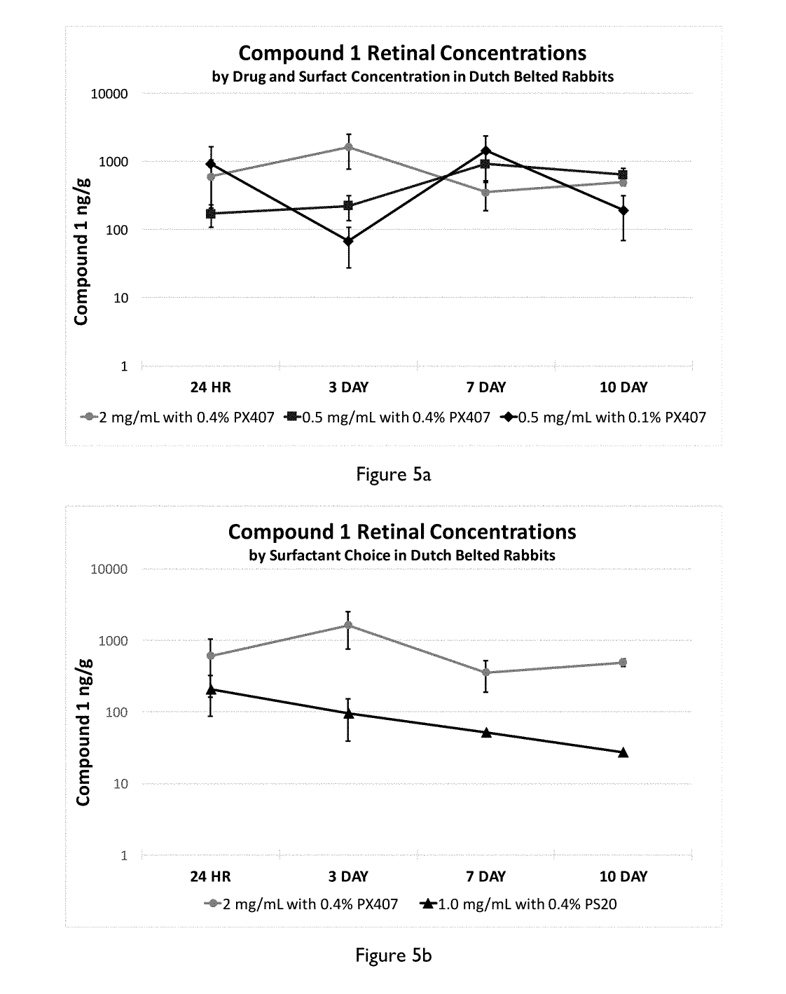

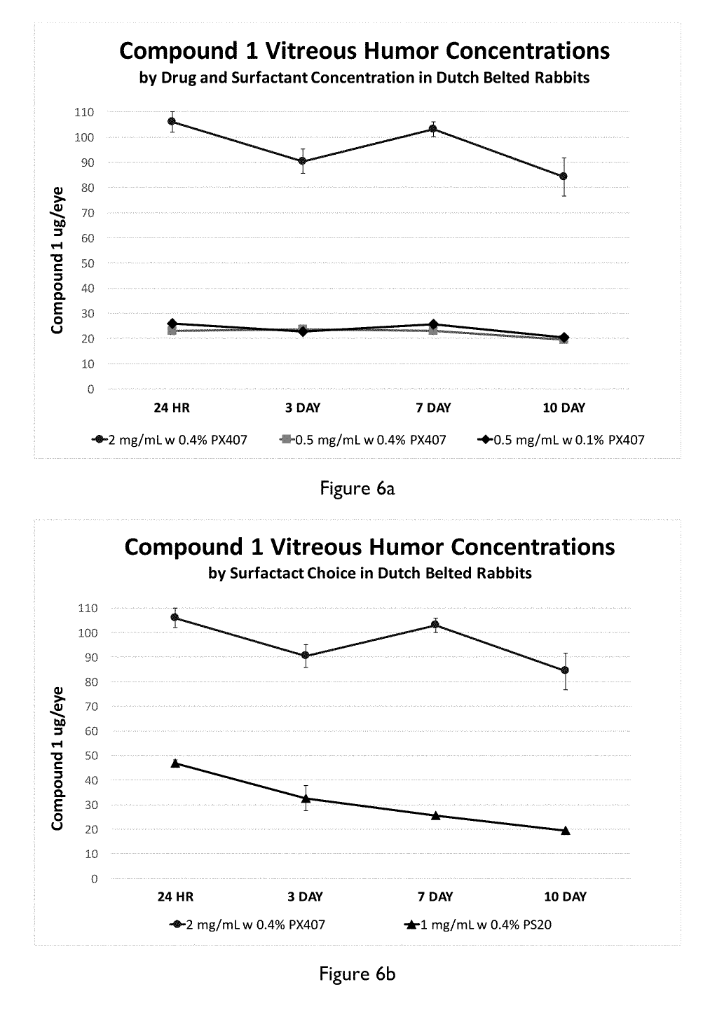

Results of this study in both retina and vitreous humor are shown in FIG. 5 (A+B) and FIG. 6 (A+B) respectively. Retinal concentrations of Compound 1 were variable with coefficient of variability (% CV) ranging from 52 to 156 percent over the time-course of the study for all groups. Retina Tₘₐₓ was noted at 72 (3 days) or 168 (7 days) hours post-administration for the three Poloxamer formulations, but was not dose dependent, while retina Tₘₐₓ for the Polysorbate formulation was 24 hours post-administration. Retina AUC₀₋ₗₐₛₜ followed a similar variable pattern as Cₘₐₓ. Retina T_{1/2} for Compound 1 could only be calculated for the 2 mg/mL Poloxamer (168 hours) and 1 mg/mL Polysorbate (199 hours) retina data.

Analysis of vitreous humor for Compound 1 indicated the concentration to be relatively consistent within each timepoint for the Poloxamer formulations. When normalized by weight of the VH collected, a theoretical total of Compound 1 was calculated. The total amount of Compound 1 injected into each rabbit eye was 100 μg (2 mg/mL*50 μL) or 25 μg (0.5 mg/mL*50 μL) for the Poloxamers and 50 μg (1 mg/mL*50 μL) for the Polysorbate group. The lowest mean concentration for all groups was at 10 days. Mean Compound 1 remaining in the VH for Group 1 (100 μg) ranged from 84.2 μg to 106 μg 24 hours to 10 days post-IVT administration. Mean Compound 1 remaining in the VH for Group 2 (25 μg) ranged from 19.5 μg to 23.0 μg 24 hours to 10 days post-IVT administration. Mean Compound 1 remaining in the VH for Group 3 (25 μg) ranged from 26.0 μg to 20.4 μg 24 hours to 10 days post-IVT administration. Mean Compound 1 remaining in the VH for the Polysorbate Group 4 (50 μg) ranged from a high of 46.8 μg at 24 hours to 19.4 μg at 10 days post-IVT administration. The Polysorbate group was the only formulation with a notable decrease in Compound 1 concentrations with time. Yet, in all groups substantial amounts of intact drug were detected 10 days post-administration.

Calculation of pharmacokinetic parameters for Compound 1 in VH indicated a Tₘₐₓ of 24 or 72 hours post IVT administration with a Cₘₐₓ closely matching the total amount of drug administered for each group (Table 4). AUC₀₋ₗₐₛₜ was nearly dose proportional between the Poloxamer groups with Group 1 (2 mg/mL) having an approximate four times greater AUC than that of Groups 2 and 3 (both 0.5 mg/mL). The Polysorbate group (1 mg/mL) was less than dose proportional relative to the Poloxamer groups but it can be readily explained as it was the only group that had a notable decrease in Compound 1 VH concentration over the course of the study. T_{1/2} for the Polysorbate group was 183 hours with a good linear fit whereas the t_{1/2} for the Poloaxmer groups was >900 hours with a less than optimal linear fit.

TABLE 4
Pharmacokinetic Parameters for Compound 1 in Vitreous Humor.

| | | | 0.5 mg/mL | |
|---|---|---|---|---|
| | 2 mg/mL | 0.5 mg/mL | Reduced | 1 mg/mL |
| Parameter | Poloxamer | Poloxamer | Poloxamer | Polysorbate |
| Tₘₐₓ (h) | 24 | 72 | 24 | 24 |
| Cₘₐₓ (μg/eye) | 106 | 23.6 | 26.0 | 46.8 |
| Terminal t_{1/2} (h) | 1028* | 994* | 911* | 183 |
| AUC₀₋ₗₐₛₜ | 22000 | 5150 | 5460 | 6870 |
| (μg*h/eye) | | | | |

*R2 < 0.7

TABLE 5
Pharmacokinetic Parameters for Compound 1 in Retina.

| | | | 0.5 mg/mL | |
|---|---|---|---|---|
| | 2 mg/mL | 0.5 mg/mL | Reduced | 1 mg/mL |
| Parameter | Poloxamer | Poloxamer | Poloxamer | Polysorbate |
| Tₘₐₓ (h) | 72 | 168 | 168 | 24 |
| Cₘₐₓ (ng/g) | 1630 | 912 | 1924 | 275 |
| Terminal t_{1/2} (h) | 168 | NC | NC | 199 |
| AUC₀₋ₗₐₛₜ (ng*h/g) | 203000 | 129000 | 219000 | 44600 |

NC—not calculated

In summary, the Compound 1 when administered IVT in either the Poloxamer or Polysorbate formulations showed no signs of irritation or tolerability issues over the 10 day course of the study. The Compound 1 when administered IVT as a Poloxamer formulation does not readily diminish in concentration for a period of up to 10 days, and likely longer, in either the retina or VH. The Compound 1 when administered as a Polysorbate formulation demonstrated a clear albeit slow decrease in concentration over the 10 day study time course, suggesting that intravitreal pharmacokinetics may be controllable within certain parameters by careful choice of the non-ionic surfactant used.

### Example 9: Rat Ocular Pharmacokinetic Study with Compound 1

This study was conducted to determine the concentrations of Compound 1 trihydrochloride in vitreous humor and retina tissues following intravitreal injections of Brown Norway rats. Concentrations were determined in tissues at 24 (Group 1), and 72 hours (Groups 1 and 2) post-dose of a 5 μL bilateral intravitreal (IVT) dose.

The study design is summarized in Table 6.

TABLE 6
Study design.

| | | | Terminal Timepoints | | |
|---|---|---|---|---|---|
| | | Dose and | (Blood and Ocular | Matrices | Matrices |
| Group | Formulation | Route | Tissues) | Collected | Analyzed |
| 1 (n = 8) | 0.06 mg/mL | 0.3 μg/eye | 24 and 72 hours | Vitreous Humor | Vitreous Humor |
| | Compound 1 in 4.5% | IVT | post-dose | and Retina | and Retina |
| | mannitol, 10 mM | | (n = 4/time point) | | |
| | acetic acid, 0.4% | | | | |
| | poloxamer 407, pH 4.5 | | | | |
| 2 (n = 8) | 0.06 mg/mL | 0.3 μg/eye | 72 hours post-dose | Vitreous Humor | Vitreous Humor |
| | Compound 1 in 4.5% | IVT | The retinas from | and Retina | and Retina |
| | mannitol, 10 mM | | 4 animals will be | | |
| | acetic acid, 0.4% | | pooled in 2 | | |
| | poloxamer 407, pH 4.5 | | samples at 72 | | |
| | | | hours. | | |

Test system included the following:
Species/Strain/Gender: Norway Brown Rats
Supplier: Charles River, Inc.
Age Range: 1 to 2 months
Weight at Receipt (Range of Weights): 165.9-181.2 g
Administration Route: Intravitreal (IVT) injection
Duration of Treatment: Single dose per eye
Formulation Concentrations: 0.06 mg/mL ONL-1204 triacetate
Dose Volume: 5 μL per eye

Harvesting of Ocular Tissues

Rats were euthanized by intravenous barbiturate overdose., and eyes were enucleated and snap frozen. Vitreous humor (VH) and retina were collected from all animals and analyzed for Compound 1 by LC-MS/MS.

Results

Ocular Tissue Concentrations

Compound 1 Trihydrochloride Concentrations in Brown Norway Rat Retina and Vitreous Humor Unknowns

TABLE 7
Concentrations of Compound 1 in Brown Norway rat retina.

| | | | | | Amount | | | | | | |
|---|---|---|---|---|---|---|---|---|---|---|---|
| | | | | | of | | | | | | |
| | | | | | Diluent | Total | Calculated | Calculated | | | |
| | Sample | Time | | Retina | Added | Volume | Concentration | Concentration | Mean | | |
| Group | ID | Point | Eye | Mass (g) | (μL) | (μL)¹ | (ng/mL) | (ng/g) | (ng/g) | SD | % CV |
| 1 | A | 24 hr | OD and OS | 0.01584 | 63 | 79 | 34.6 | 173 | 1210 | 1670 | 138 |
| 0.06 mg/mL | B | | OD and OS | 0.02249 | 90 | 112 | 736 | 3670 | | | |
| Compound 1 | C | | OD and OS | 0.01744 | 70 | 87 | 29.2 | 146 | | | |
| in 4.5% | D | | OD and OS | 0.01899 | 76 | 95 | 172 | 860 | | | |
| mannitol, 10 | A | 72 hr | OD and OS | 0.01395 | 56 | 70 | 81.5 | 409 | 570 | 172 | 30.2 |
| mM acetic | B | | OD and OS | 0.01943 | 78 | 97 | 117 | 584 | | | |
| acid, 0.4% | C | | OD and OS | 0.01514 | 61 | 76 | 96.0 | 482 | | | |
| poloxamer | D | | OD and OS | 0.01742 | 70 | 87 | 161 | 804 | | | |
| 407, pH 4.5 | | | | | | | | | | | |
| 2 | A-D | 72 hr | All pooled | 0.06088 | 244 | 305 | 92.2 | 462 | NA | NA | NA |
| 0.06 mg/mL | E-H | 72 hr | All pooled | 0.04906 | 196 | 245 | 262 | 1310 | NA | NA | NA |
| Compound 1 | | | | | | | | | | | |
| in 4.5% | | | | | | | | | | | |
| mannitol, 10 | | | | | | | | | | | |
| mM acetic | | | | | | | | | | | |
| acid, 0.4% | | | | | | | | | | | |
| poloxamer | | | | | | | | | | | |
| 407, pH 4.5 | | | | | | | | | | | |

LLOQ = 20.0 ng/mL or 100 ng/g
Values < LLOQ use 0 for statistical determination
Footnote 1: 1.00 g/mL is assumed for rat retina tissue

TABLE 8
Concentrations of Compound 1 in Brown Norway rat vitreous humor.

| | | | | | Vitreous | Calculated | | | |
|---|---|---|---|---|---|---|---|---|---|
| | | | | Calculated | Humor | Concentration | Mean | | |
| | Sample | Time | | Concentration | Weight | (μg/VH | (μg/VH | | % |
| Group | ID | Point | Eye | (ng/mL) | (g)* | sample) | sample) | SD | CV |
| 1 | A | 24 hr | OD and OS | 1780 | 0.05314 | 0.0946 | 0.157 | 0.0568 | 36.2 |
| 0.06 mg/mL | B | | OD and OS | 5210 | 0.03262 | 0.170 | | | |
| Compound | C | | OD and OS | 4360 | 0.05262 | 0.229 | | | |
| 1 in 4.5% | D | | OD and OS | 3620 | 0.03768 | 0.136 | | | |
| mannitol, 10 | A | 72 hr | OD and OS | 1480 | 0.04818 | 0.0713 | 0.0994 | 0.0738 | 74.2 |
| mM acetic | B | | OD and OS | 3740 | 0.05013 | 0.187 | | | |
| acid, 0.4% | C | | OD and OS | 306 | 0.04681 | 0.0143 | | | |
| poloxamer | D | | OD and OS | 2350 | 0.05314 | 0.125 | | | |
| 407, pH 4.5 | | | | | | | | | |
| 2 | A | 72 hr | OD and OS | 3030 | 0.04931 | 0.149 | 0.127 | 0.0836 | 65.8 |
| 0.06 mg/mL | B | | OD and OS | 453 | 0.05496 | 0.0249 | | | |
| Compound | C | | OD and OS | 3590 | 0.05423 | 0.195 | | | |
| 1 in 4.5% | D | | OD and OS | 3610 | 0.05280 | 0.191 | | | |
| mannitol, 10 | E | | OD and OS | 2910 | 0.04381 | 0.127 | | | |
| mM acetic | F | | OD and OS | 4070 | 0.05832 | 0.237 | | | |
| acid, 0.4% | G | | OD and OS | 69.0 | 0.02919 | 0.00201 | | | |
| poloxamer | H | | OD and OS | 4800 | 0.01807 | 0.0867 | | | |
| 407, pH 4.5 | | | | | | | | | |

LLOQ = 50.0 ng/mL
*assume density of 1.00 g/mL

Tissue Homogenization

Instructions for Homogenization of Vitreous Humor (VH) Unknowns

For each VH unknown or control blank: Weigh VH into a homogenization tube. Add 4 times the VH weight (mg) of ACN:water:1 M hydrochloric acid (70:20:10, v/v/v) (μL) to the homogenization tube. Add zirconium oxide beads, 2.8 and 1.4 mm size. Homogenize all samples on Precellys®: 5500 rpm, 3×30 second cycles, and 20 seconds between cycles, at a temperature between −10 to 0° C.

Instructions for Homogenization of Retina Ocular Tissue Standards

For Each Retina Standard:

Weigh blank retina tissue into a homogenization tube.

Add 0.5 times the tissue weight (mg) of Retina Working Calibration Standard (μL) to the homogenization tube.

Add 3.5 times the tissue weight (mg) of ACN:water:1 M hydrochloric acid (70:20:10, v/v/v) (μL) to the homogenization tube.

Add zirconium oxide beads, 1.4 mm size.

Homogenize all samples on Precellys®: 5500 rpm, 3×30 second cycles, and 20 seconds between cycles, at a temperature between −10 to 0° C.

Instructions for Homogenization of Retina Tissue Blank Controls, and Unknowns

For each retina unknown or control blank:

Weigh retina tissue into a homogenization tube.

Add 4 times the tissue weight (mg) of ACN:water:1 M hydrochloric acid (70:20:10, v/v/v) (μL) to the homogenization tube.

Add zirconium oxide beads, 1.4 mm size.

Homogenize all samples on Precellys®: 5500 rpm, 3×30 second cycles, and 20 seconds between cycles, at a temperature between −10 to 0° C.

Preparation of Standards, Samples, and Blanks

Preparation of Calibration Stock and Working Standards

A stock calibration standard was prepared in dimethylsulfoxide (DMSO) at a concentration of 500 μg/mL for ONL-1204.

Working calibration standards were prepared for vitreous humor by serial dilution of working stock solution with ACN:wWater:1M hydrochloric acid (70:20:10, v/v/v) over a range of 500 ng/mL to 200,000 ng/mL ONL-1204.

Working calibration standards were prepared for retina by serial dilution of working stock solution with acetonitrile:water:1 M hydrochloric acid (70:20:10) over a range of 100 ng/mL to 200,000 ng/mL ONL-1204.

Preparation of Standards, Unknowns, Blanks, and Blanks with Internal Standard for Vitreous Humor Analysis

In a polypropylene tube, ten (10) μL (20 μL STD 11) of working calibration standard or stock was added to 90 μL (80 μL STD 11) control blank vitreous humor. For blanks and blanks with internal standard, 100 μL of control blank Bovine vitreous humor was added. Four hundred (400) μL of ACN:water:1M hydrochloric acid (70:20:10, v/v/v) was added to each standard or blank.

One hundred (100) μL of each vitreous humor sample with ACN: formic acid (1000:1, v/v) was then aliquoted. One hundred (100) μL of DMSO:water:formic acid (50:40:10) was added to each vitreous humor sample. The samples were vortex mixed then centrifuged for 10 minutes at 14,000 rpm (4° C.). To 50.0 μL supernatant, 100 μL of working internal standard (50,000 ng/mL APi 1887 in water) (water for the blank without internal standard), and 150 μL of water were added. The samples were then vortex mixed and transferred to an autosampler plate for analysis.

Preparation of Standards, Unknowns, Blanks, and Blanks with Internal Standard for Retina Analysis

In a polypropylene tube, 50 μL of Brown Norway rat unknown homogenate, bovine control blank or calibration standard Bovine homogenate was added. Fifty (50) μL of DMSO:water:formic acid (50:40:10) was added to each sample. The samples were then vortex mixed and centrifuged for 10 minutes at 14,000 rpm (4° C.). Eighty (80) μL of each sample supernatant was then aliquoted to a 96-well autosampler plate. Forty (40) μL of working internal standard (5,000 ng/mL APi 1887 in water) (water for the blank without internal standard), and 120 μL of water were added. The samples were then mixed with a multichannel pipette and analyzed.

Calculations

Percent Coefficient of Variation:

Used as an estimate of precision. Percent Coefficient of Variation (% CV)=(Standard Deviation/average value)*100

Quadratic Least Squares Analysis:

The standard curve fit was determined using a quadratic equation with 1/x² weighting:

y=ax²+bx+c

where: y=peak area ratio of the calibration standards to internal standard
x=concentration of the calibration standard
a=quadratic coefficient of x²
b=quadratic coefficient of x
c=the constant as the y-intercept of the calibration curve

Quadratic Analyte Concentration:

The concentration of analyte is calculated using the calibration curve parameters calculated above and then solving for the value of x.

Analysis

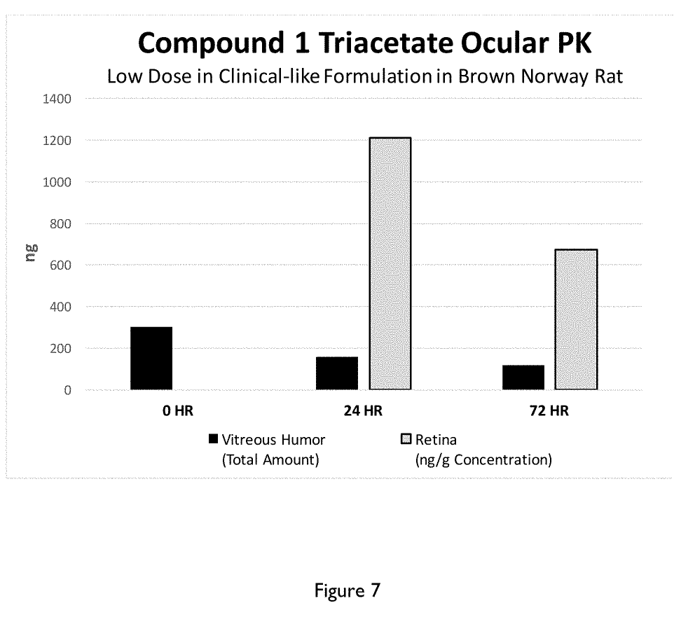

The globally averaged results are shown in FIG. 7. For Group 1 both eyes from each animal were combined for a single analysis. In Group 2 both eyes were combined from 4 animals to make samples. Concentrations for the pooled retina in Group 2 were 462 and 1310 ng/g for the A-D and E-H samples respectively. Analysis of vitreous humor for Compound 1 was conducted using combined samples from each eye for each animal in Groups 1 and 2. When normalized by weight of the VH collected, a theoretical total of Compound 1 was calculated. The total amount of Compound 1 injected into each rat eye was 0.3 μg (0.06 mg/mL*5 μL)). Because of the different ways the 72 hour data was collected, standard error was not calculated. The dark bars represent the amount of Compound 1 triacetate in the vitreous humor of each eye, with the t=0 bar representing the intended dose of 300 ng, and the light bars represent the concentration of Compound 1 in the retina expressed in ng/g. In the first 24 hours, about half the drug cleared the VH, but the terminal half-life was clearly on the order of several days. Meanwhile, retinal concentrations were above 1 μg/g at 24 hrs, and have only about 40% by 72 hrs. This suggests that the rat retina will be exposed to the drug in readily detectable amounts for at least a week.

Retinal concentrations of Compound 1 ranged from 146 to 3670 ng/g for the Group 124 hour samples, and from 409 to 804 ng/g in the Group 172 hour samples. Mean Compound 1 remaining in the VH for Group 1 was 0.157 and 0.0994 μg/sample at the 24 hour and 72 hour time points respectively. Mean Compound 1 remaining in the VH for Group 2 samples at 72 hours was 0.127 μg/sample. The data indicates that substantial amounts of intact drug remain 72 hours post-administration.

### Example 10: In Vivo Efficacy of Compound 1

Briefly, rodents were anesthetized with a 50:50 mix of ketamine (100 mg/mL) and xylazine (20 mg/mL), and pupils were dilated with topical phenylephrine (2.5%) and tropicamide (1%). A 20-gauge microvitreoretinal blade (Walcott Scientific, Marmora, N.J.) was used to create a sclerotomy 2 mm posterior to the limbus, carefully avoiding lens damage.

Under direct visualization through an operating microscope, a subretinal injector (Glaser, 32-gauge tip; BD Ophthalmic Systems, Sarasota, Fla.) was introduced through the sclerotomy into the vitreous cavity and then through a peripheral retinotomy into the subretinal space. Sodium hyaluronate (10 mg/mL) was slowly injected to detach the neurosensory retina from the underlying retinal pigment epithelium.

In all experiments, approximately one-third to one-half of the superonasal neurosensory retina was detached. In all animals, detachments were created in the same location to allow for direct comparison of retinal cell counts. Detachments were created in the left eye, leaving the right eye as the control.

In some eyes, wild-type Met-12 (HHIYLGAVNYIY, 5 μg in DMSO) as its trihydrochloride salt was given as a positive control, and in other eyes, Compound 1 (0.5, 1.0, 5.0 or 10 μg in DMSO) as its trihydrochloride salt, or vehicle (dimethyl sulfoxide [DMSO]) was injected into the subretinal space in the area of the detachment in a 5-μL volume using a Hamilton syringe (Hamilton Company, Reno, Nev.) immediately after the creation of the detachment.

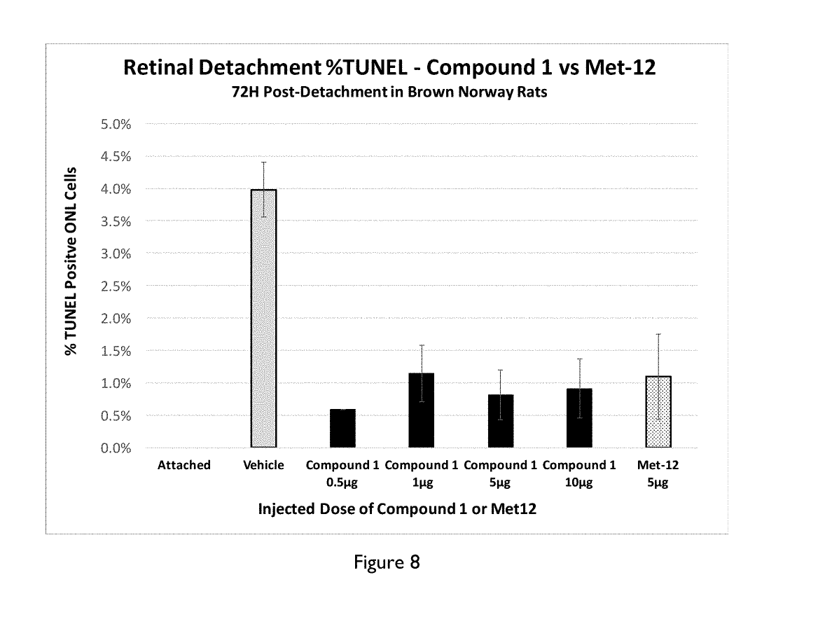

After three days rats were euthanized, retinas were excised, fixed, sectioned, and stained for TUNEL assays. Areas of detached retina were counted for number of apoptotic cells. Each experiment involved 4 fields from each of 4 sections obtained from 5 or 6 retinas for each dosing group. The results are shown in FIG. 8.

As shown in FIG. 8, control attached retinas showed no apoptotic cells, whereas DMSO control retinas have approximately 4% of cells staining for TUNEL, and the positive control Met-12 (5 μg) with just over 1% positive for TUNEL. Compound 1 trihydrochloride salt treatment led to 0.58% at 0.5 μg, 1.14% at 1 μg, 0.82% at 5 μg, and 0.9% at 10 μg. Based on considerable experience with the model, the results for Compound 1 are considered approximately equivalent with each other and with the Met-12 5 μg positive control. Thus unexpectedly, Compound 1 trihydrochloride salt is much more efficacious than Met-12 trihydrochloride salt in this in vivo model.

### Example 11: Efficacy Study

Using the same rodent retinal detachment model as in Example 10, the highly efficacious 5 μg dose of Compound 1 in DMSO was compared with the same dose, and a 1 μg dose of Compound 1 in two different formulations.

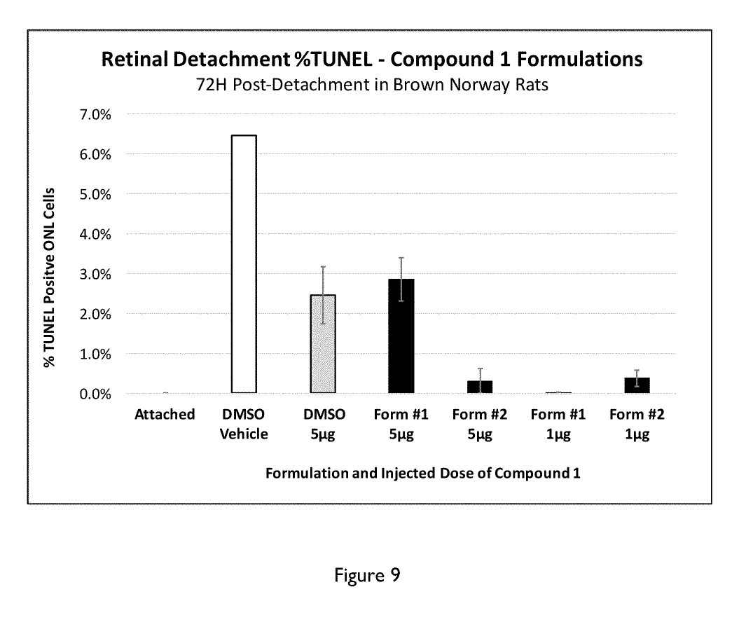

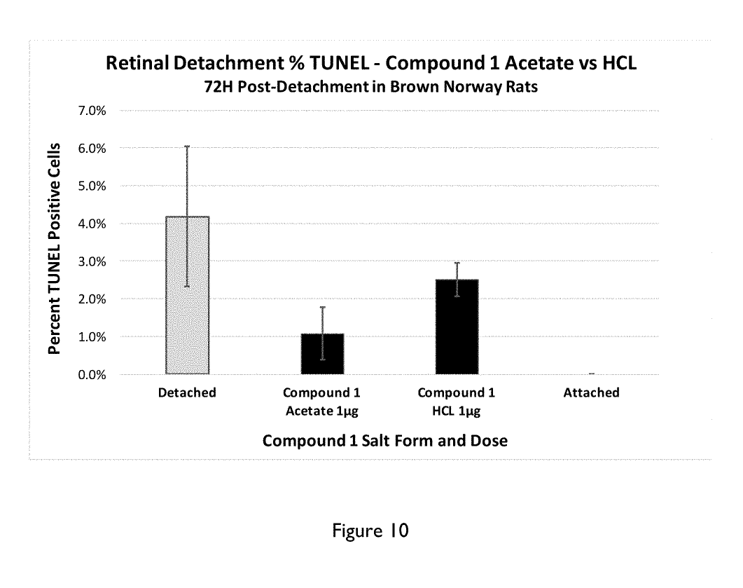

The first formulation was a 1.0 or 0.2 mg/mL solution of Compound 1 trihydrochloride in 3% propylene glycol and 3% PS-20 at pH 4.0, and the second formulation was the same concentrations of Compound 1 in a 2% propylene glycol 2% poloxamer 407 solution, also at pH 4. The results are shown in FIG. 9.

The attached control retinas showed no apoptotic cells whereas the untreated detachments showed approximately 6.5% apoptotic cells as measured at this time point.

Compound 1 in DMSO reduced that to 2.5%, and the 5 μg PG/PS-20 was of similar potency reducing the apoptotic cells to 2.9%. However the PG/PX formulation was considerably better at that concentration reducing the apoptotic cells to 0.3% at 5 μg. At 1 μg the PG/PX formulation reduced apoptotic cells to 0.4%, but the PG/PS-20 1 μg dose produced an even greater lowering to 0.01%. The data demonstrates that not only can micellar formations work, but that Compound 1 trihydrochloride salt can be even more potent in them than when it is formulated in DMSO.

### Example 12: Efficacy Study

Using the same rodent retinal detachment model as in Example 10, Compound 1 trihydrochloride salt in DMSO (1 μg) was used as the positive control. The negative control was a test vehicle under evaluation (0.4% Poloxamer, 4.5% mannitol, 10 mM acetic acid at pH 4.5.) Compound 1 triacetate salt in DMSO (1 μg) was compared to the trihydrochloride salt at the same dose.

The attached retinas (Bar 4) had no apoptotic cells, whereas vehicle treated detached retinas (Bar 1) showed 4.2% apoptotic cells, which suggests no activity, as it is within the historic range for untreated retinal detachments (See example 10). The Compound 1 trihydrochloride salt (DMSO) gave 2.4% apoptotic cells, whereas the triacetate salt (DMSO) gave only 1.2%, demonstrating that the switch from hydrochloride to acetate salts does not have negative effects, and quite possibly positive effects on efficacy.

### Example 13

The goal of this study was to analyze the tissue samples for changes in gene expression following elevation of intraocular pressure (“IOP”) in the presence or absence of Compound 1.

Methods:

Quantitative PCR (qPCR) was used on neural retina samples isolated at 28 days post microbead or saline injection from mice treated with Compound 1 (or vehicle) on Day 0.

Data shown are mRNA expression fold change over saline+vehicle control+/−SEM. N=6/group, **P<0.01, ***P<0.001, ****P<0.0001.

A 96-well was expanded to a 384-well qPCR system to allow for an increase in the number of genes to be examined in one run.

Also, new house keeping genes were tested and validated, as the house keeping genes, beta actin and HPRT1, that were used in our previous studies, proved to be unreliable and showed variable expression levels between our experimental groups.

After testing several retina house keeping genes, it was found that B2-microglobulin (B2M) and peptidylprolyl isomerase A (PPIA) were both very stable between all experimental groups and the average Ct-values for both house keeping genes were used to calculate DCt in these studies.

For this qPCR analysis, saline+vehicle was used as the control. DDCt=experimental DCt−mean DCt of saline+vehicle, and Expression Fold Change=2\hat{ }−DDCt.

Results:

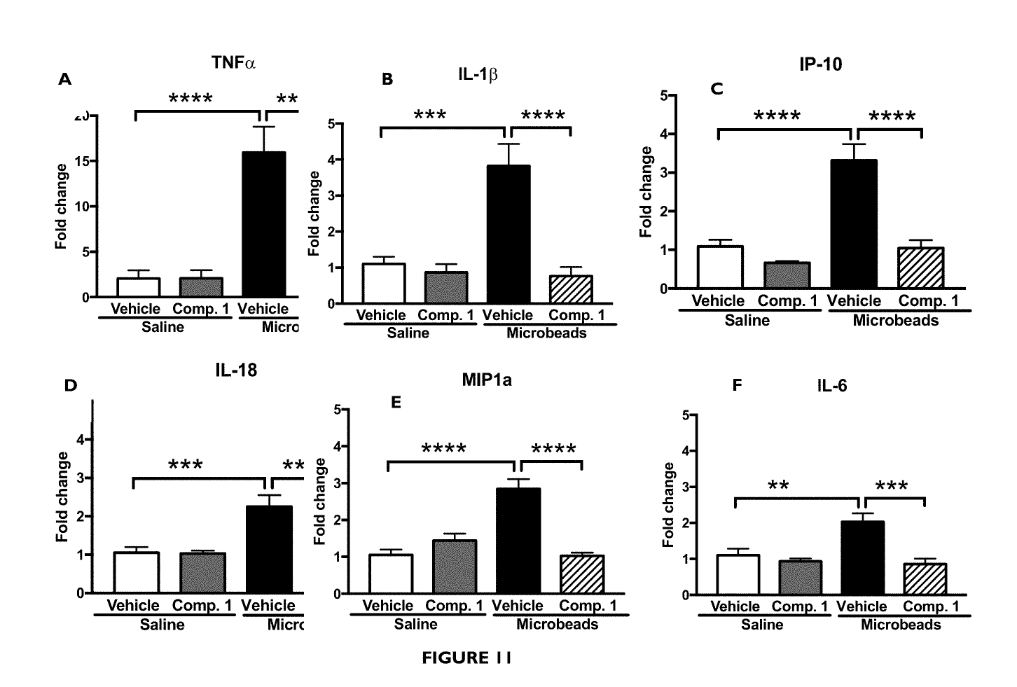

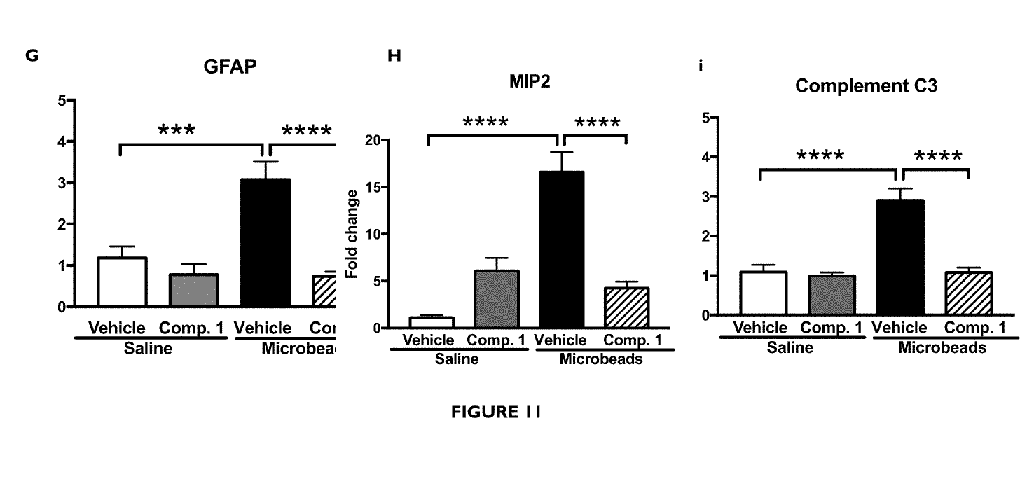

As shown in FIGS. 11 and 12, animals that were injected with microbeads and vehicle exhibited significantly higher expression of the inflammation-related genes, TNFα (FIG. 11A), IL-1β (FIG. 11B), IP-10 (FIG. 11C), IL-18 (FIG. 11D), MIP1α (FIG. 11E), IL-6 (FIG. 11F), GFAP (FIG. 11G), MIP2 (FIG. 11H), MCP-1 (FIG. 12A), and MIP-1β (FIG. 12E). The expression of these genes was significantly reduced in animals treated with Compound 1.

Following elevated IOP, the gene expression of the complement-related proteins C1q (FIG. 12G) and Complement C3 (FIG. 11i) were also significantly increased. As seen with the expression of the other inflammatory genes, the expression of C3 and C1q was significantly reduced in the animals treated with Compound 1.

Additional genes (Caspase 8, NLRP3, TLR4) were increased following elevated IOP (see FIG. 12B, FIG. 12F and FIG. 12D), but the increase did not reach significance when comparing the microbead group to the saline control group. However, the expression of all three genes were significantly reduced in the microbead injected animals treated with Compound 1.

The expression of cFLIP (FIG. 12C), generally considered to be pro-survival, was decreased in the microbead/saline animals, and restored to near-baseline in the Compound 1 treated animals.

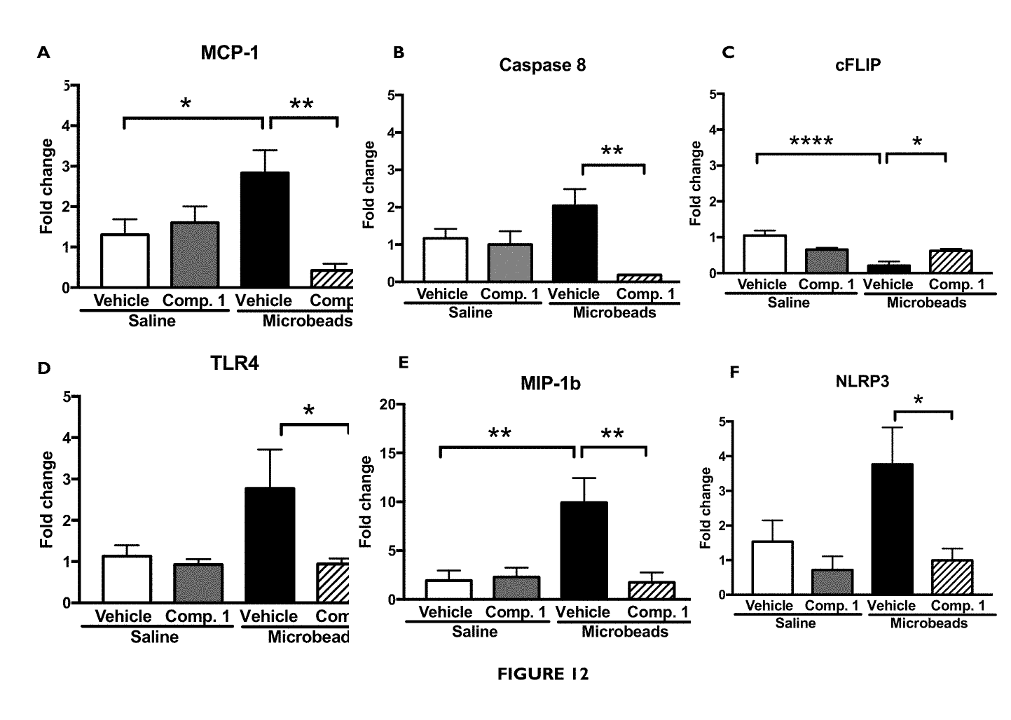

As shown in FIG. 13, other genes related to apoptosis, including Bax (FIG. 13A), FADD (FIG. 13B), FasR (FIG. 13D), FasL (FIG. 13E), and caspase 3 (FIG. 13H), were unchanged following elevated IOP and appeared to be unaffected by Compound 1 at this 28 day time point. Limited or no change was observed in some other inflammation-related genes, including ASC (FIG. 13C), NLRP2 (FIG. 13G), and complement C4 (FIG. 13F).

As described previously, Fas has been known to induce inflammatory signaling that propagate cell death and tissue damage. These data demonstrate that Fas inhibition by Compound 1 reduces the expression of inflammatory genes following elevated IOP, thereby preventing and/or reducing the inflammatory microenvironment induced by elevated IOP.

Additionally, the observation that the expression of complement factors C3 and C1q were significantly elevated with microbead injection and were significantly reduced with Compound 1 treatment, suggests that Fas is upstream of complement signaling.

Taken together, these observations suggest that Fas is upstream of a host of inflammatory mediators, and inhibition of one of these downstream factors may not prevent the overall inflammatory microenvironment as effectively as inhibiting Fas.

These data support the potential of Compound 1 and Fas inhibition as part of a therapeutic strategy for treatment of glaucoma.

### Example 14

The goals of this study were to determine whether the Fas inhibitor, Compound 1 can prevent the death of retinal ganglion cells (RGCs) and axons in the microbead-induced mouse model of elevated IOP and to evaluate if Fas inhibition can down-modulate the inflammatory microenvironment.

Methods:

All animal experiments were approved by the Institutional Animal Care and Use Committee at Schepens Eye Research Institute and were performed under the guidelines of the Association of Research in Vision and Ophthalmology (Rockville, Md.).

C57BL/6J mice were used in this experiment in which 2 μL of sterile polystyrene microbeads (15 μm; 7.2×10⁶ bead/mL) or saline were injected into the anterior chamber on Day 0 followed by 1 μL of 0.5 mg/mL or 2 mg/mL Compound 1 or vehicle by intravitreal (IVT) injection on Day 0 or 7 days after the microbead/saline injections. IOP was followed every 3 days for 4 weeks using a rebound tonometer (TonoLab). At 4 weeks post anterior chamber injection, retinal flatmounts were prepared and stained for Brn3a, an RGC-specific protein, to visualize RGCs. Sixteen non-overlapping images were taken, at 60× with 4-5 images within each quadrant and the images were used to calculate the RGC density. For axon analysis, optic nerves were stained with p-phenylenediamine (PPD) to visualize myelinated axons and 10 non-overlapping photomicrographs were taken at 100× magnification covering the entire area of the optic nerve cross-section, and these images were used to calculate the axon density. Quantitative PCR (qPCR) was also performed on retinal tissue isolated from the mice at 28 days post microbead/saline injections. To assess production of mature IL-113 (p17), protein lysates (20 μg per sample) were prepared from posterior eye cups (neural retina, choroid, and sclera) at 28 days post microbead/saline injections and analyzed by Western blot and densitometry. All data are presented as mean±SEM. One-way ANOVA and the Sidak multiple-comparison test were used for analysis of RGCs and axons. A p value <0.05 was considered significant.

Results:

IOP:

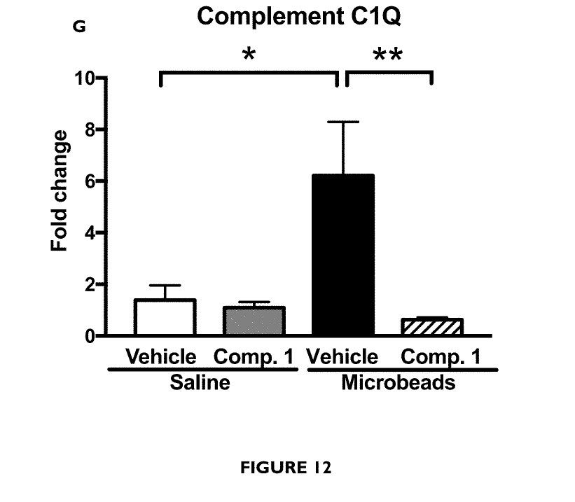

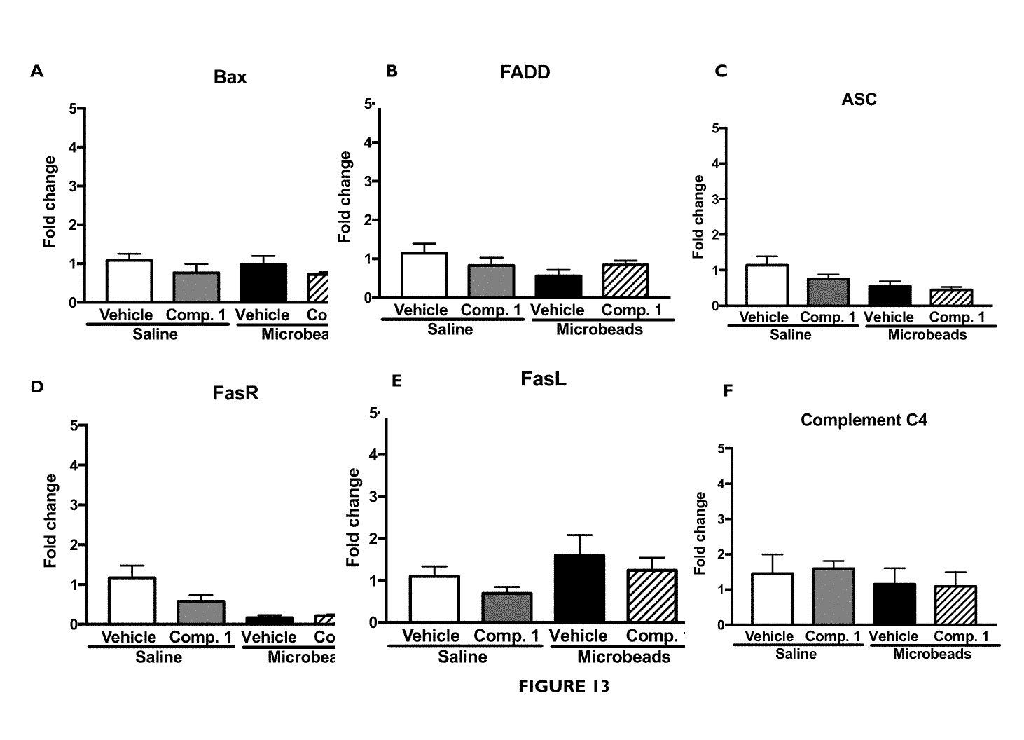

The microbead injections induced the expected increase in IOP to 20-25 mm Hg from a baseline of 15 mm Hg, peaking around day 3 or 7 post-microbead injection. Saline injection had no significant effect on IOP. IVT injection with Compound 1 did not affect IOP when administered on the same day as the microbeads (FIG. 14) or when administered on Day 7 post microbeads (FIG. 15).

RGC and Axon Counts:

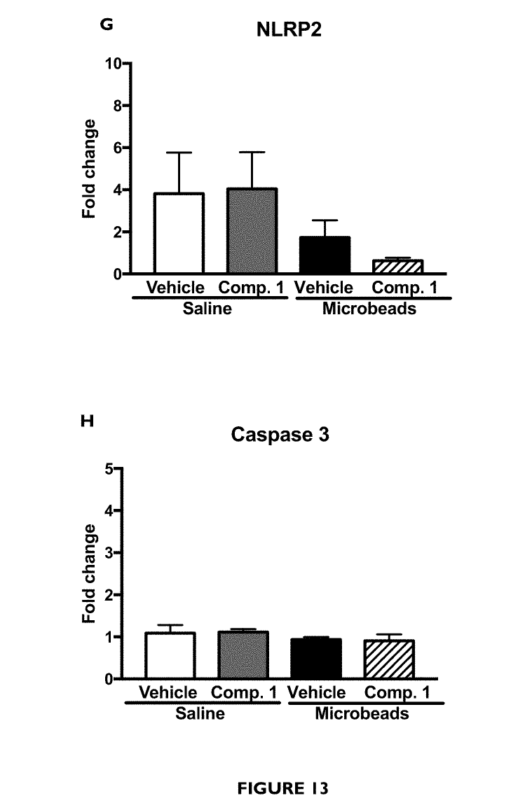

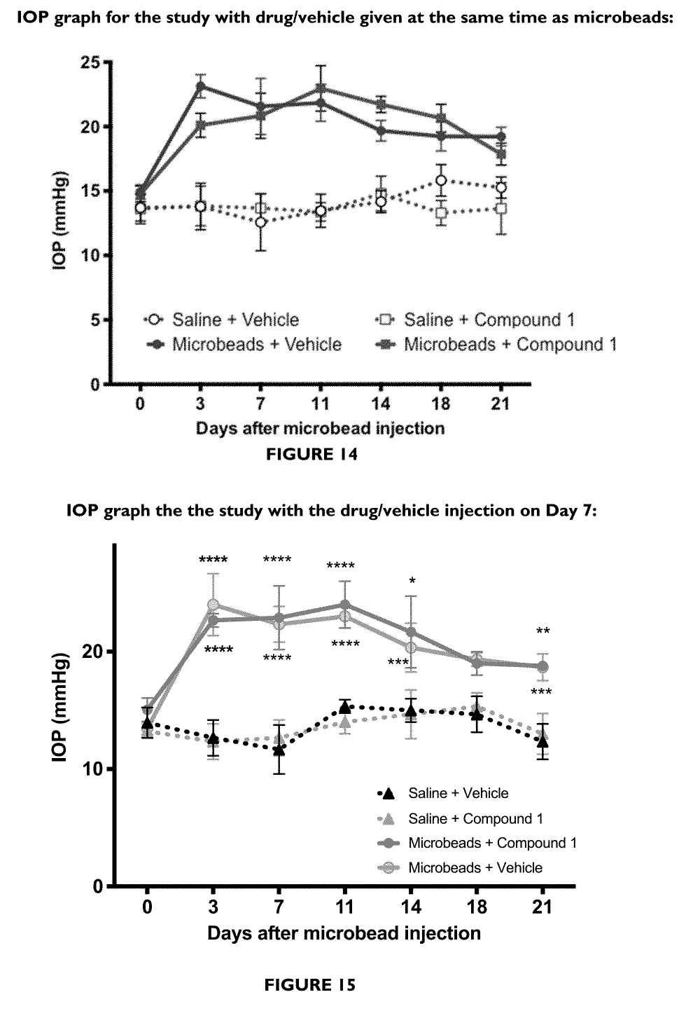

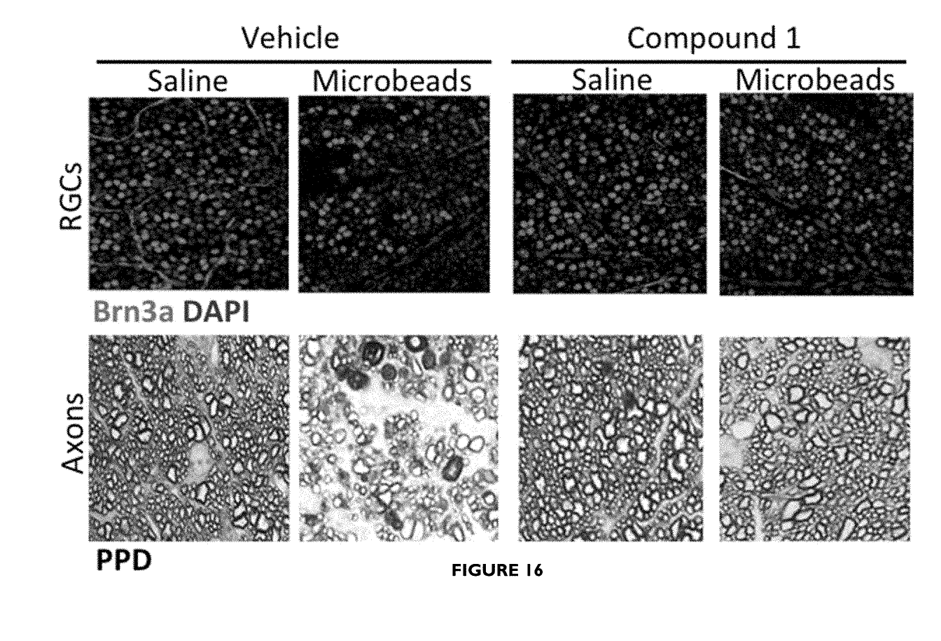

Treatment with Compound 1 at 0.5 mg/ml or 2 mg/ml achieved comparable and statistically significant preservation of retinal ganglion cell and axon density when given at Day 0. Representative images (FIG. 16) and the quantification of the total collected images are shown in FIG. 17 for RGC Cell density and FIG. 18 for Axon density.

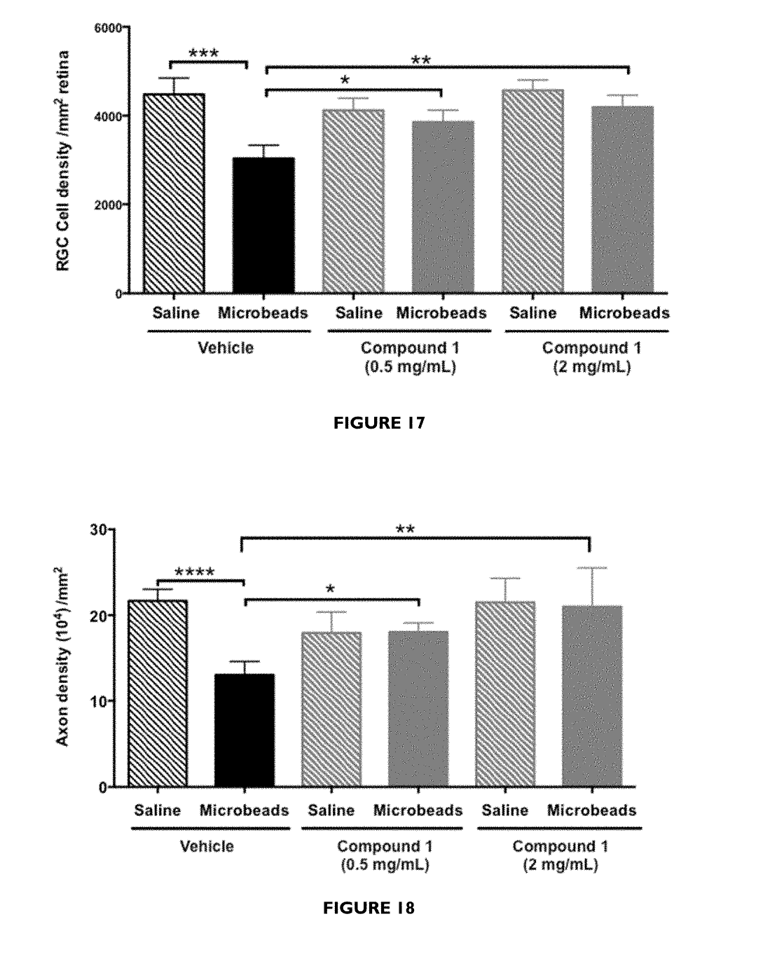

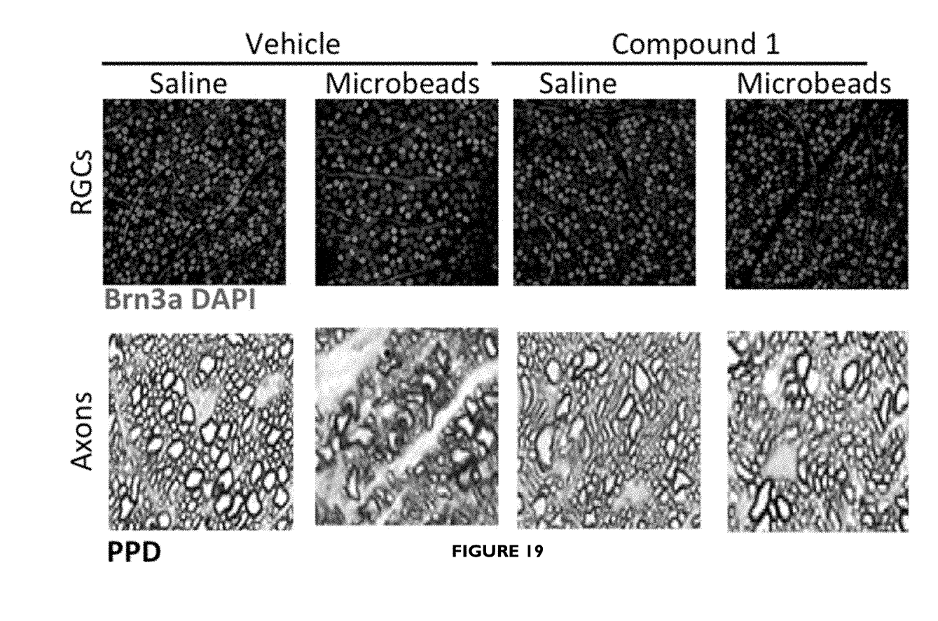

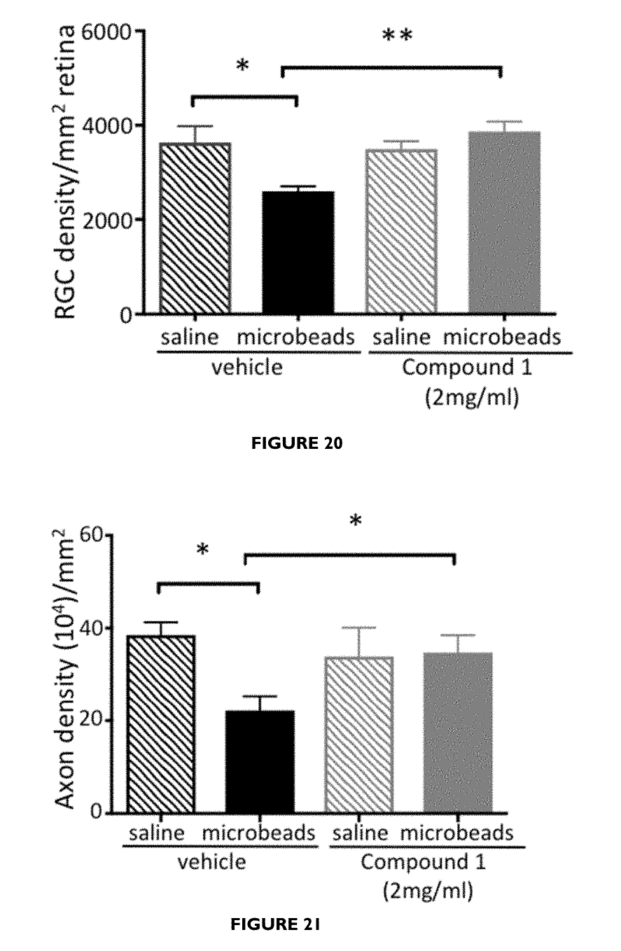

Only the 2.0 mg/mL (2 μg) dose of Compound 1 was tested at Day 7 post-microbead injection and, also, achieved nearly total preservation of retinal ganglion cell and axon density when compared to saline plus vehicle controls, as shown in the FIGS. 19, 20 and 21.

Inflammatory Microenvironment

Compound 1 inhibited microglial/macrophage activation.

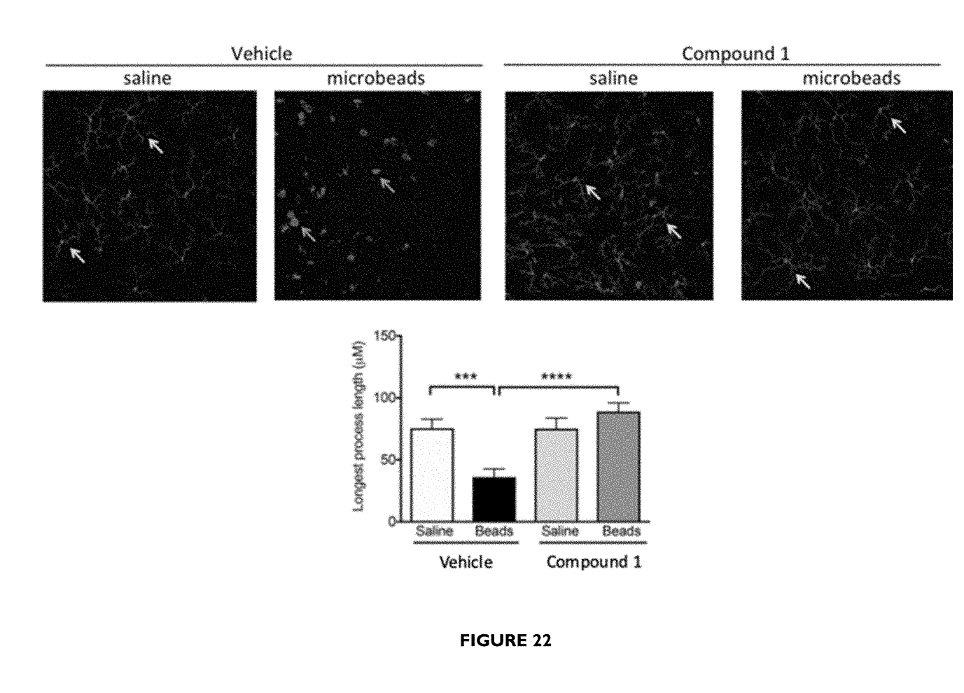

FIG. 22 depicts representative confocal images of retinal whole mounts at 28 days post microbead injection from mice treated with ONL 1204 (or vehicle) at Day 0. Retinal whole mounts were stained with Iba1 (microglia/macrophage). Yellow arrows indicate homeostatic microglia with dendritic morphology; blue arrows indicate activated microglia and/or infiltrating macrophages with amoeboid morphology. Morphometric analysis was performed on Iba1+ cells in the ganglion cell layer (60 cells per retina) and the longest process length measured from the edge of the cell body (in μm) was used to quantitate microglia activation.

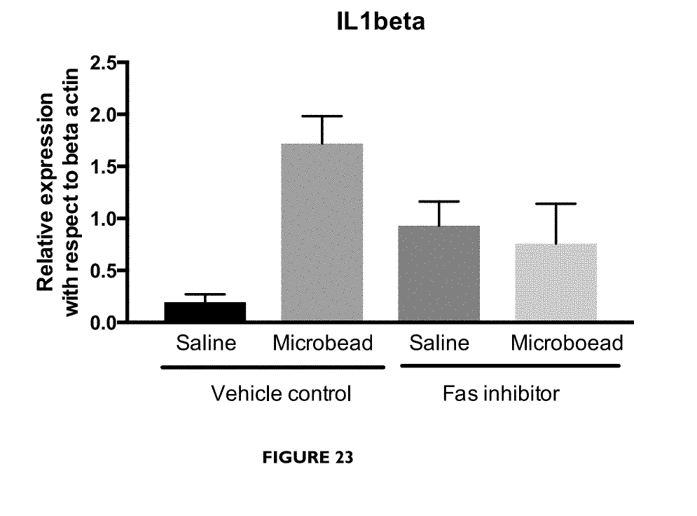

As shown in FIG. 23, additionally, treatment with Compound 1 substantially inhibited TNF-α, CCL2/MCP-1 and CCL3/MIP-1α gene expression and reduced the production of mature IL-1β. Quantitative PCR was performed on neural retina isolated at 28 days post microbead injection from mice treated with ONL 1204 (or vehicle) on Day 0. Data shown are mRNA expression fold change over saline controls+/−SEM. N=4/group, *P<0.05, **P<0.01. For analysis of mature IL-1β (p17) production, protein lysates (20 μg per sample) were prepared from posterior eye cups (neural retina, choroid, and sclera) and analyzed by Western blot. Densitometry reveals a nearly two-fold reduction in the production of mature IL-1β following microbead injection in the mice treated with Compound 1 as compared to vehicle.

### CONCLUSIONS

Based on previous showing that Fas/FasL pathway is required for death of RGCs and loss of axons in the microbead-induced mouse model of glaucoma and the role of Fas in this process, as well as previous data from our laboratory showing protection of retinal cells following treatment with Compound 1, we were interested in determining whether Compound 1 could be used as a neuroprotective therapy to protect RGCs and prevent loss of axons in the microbead model of glaucoma.

These data demonstrate that treatment with Compound 1, a small peptide inhibitor of Fas, protects RGCs and prevents axon loss in this model of elevated IOP. This protection is observed even when Compound 1 is delivered after IOP has been elevated, which is a more clinically relevant scenario.

Furthermore, since Fas is known to trigger inflammatory signaling that can lead to additional cell death and tissues damage, the effect of Compound 1 on the inflammatory microenvironment was assessed. Treatment with Compound 1 reduced the inflammatory microenvironment, as indicated by the decreased expression of inflammatory cytokines/chemokines and the reduced number of activated microglia/macrophages. These data complement the company's separate efforts showing that treatment with Compound 1 results in decreased inflammatory markers.

These data support the potential of Compound 1 and Fas inhibition as part of a therapeutic strategy in the treatment of glaucoma.

All publications and patents mentioned in the present application and/or listed below are herein incorporated by reference. Various modification and variation of the described methods and compositions of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

Although the present invention has been described in considerable detail with reference to certain embodiments thereof, other embodiments are possible without departing from the present invention. The spirit and scope of the appended claims should not be limited, therefore, to the description of the preferred embodiments contained herein. All embodiments that come within the meaning of the claims, either literally or by equivalence, are intended to be embraced therein.

Furthermore, the advantages described above are not necessarily the only advantages of the invention, and it is not necessarily expected that all of the described advantages will be achieved with every embodiment of the invention.

1. A method for preventing, treating or ameliorating an inflammation-mediated and/or complement-mediated disease or condition in a subject, comprising administering to the subject a Fas inhibitor, a derivative thereof, a fragment thereof, a pharmaceutically acceptable salt thereof, or a gene therapy encoding the Fas inhibitor, in an amount effective to inhibit Fas signaling, wherein the inhibition of Fas signaling results in at least one of the following:
reduction of expression or concentration of at least one Fas-mediated inflammation-related gene or protein;
reduction of expression or concentration of at least one Fas-mediated complement-related gene or protein;
reduction of gene or protein expression or concentration of Caspase 8;
reduction of gene or protein expression or concentration of one or more components of the inflammasome;
reduction of gene or protein expression or concentration of one or more C-X-C motif chemokines;
reduction of gene or protein expression or concentration of one or more C-X3-C motif chemokines;
reduction of gene or protein expression or concentration of one or more C-C motif chemokines;
reduction of gene or protein expression or concentration of toll-like receptor 4 (TLR4);
reduction of gene or protein expression or concentration of one or more interleukin cytokines;
reduction of gene or protein expression or concentration of one or more TNF superfamily cytokines; or
reduction of Fas-mediated Müller cell activation as indicated by reduced GFAP gene or protein expression or concentration,
thereby preventing, treating, or ameliorating the disease or condition in the subject.

2. The method of claim 1, wherein the subject has or is at risk of having the inflammation-mediated and/or complement-mediated disease or condition.

3. The method of claim 1, wherein the inflammation-mediated and/or complement-mediated disease or condition is selected from the group consisting of retinal disease, immunological disease, cancer, amyloid disease, an injury caused by ischemia or reperfusion, autoimmune disease, neurodegeneration, and diseases of the central nervous system.

4. The method of claim 1, wherein the Fas inhibitor, its derivative, the pharmaceutically acceptable salt thereof, or gene therapy is administered in a pharmaceutical composition comprising the Fas inhibitor, its derivative, pharmaceutically acceptable salt, or gene therapy; and a pharmaceutically acceptable additive.

5. The method of claim 1, wherein the Fas inhibitor, its derivative, the pharmaceutically acceptable salt thereof, or gene therapy is administered via an injection.

6. The method of claim 1, wherein the Fas-mediated inflammation-related gene or protein is selected from the group consisting of TNFα, IL-1β, IP-10, IL-18, MIP1α, IL-6, GFAP, MIP2, MCP-1, or MIP-1β.

7. The method of claim 1, wherein the at least one Fas-mediated complement-related gene or protein is complement component 3 (C3) or complement component 1q (C1q).

8. The method of claim 1, wherein the one or more components of the inflammasome is NLRP3 or NLRP2.

9. The method of claim 1, wherein the one or more C-X-C motif chemokine is CXCL2 (MIP-2α) or CXCL10 (IP-10).

10. The method of claim 1, wherein the C-X3-C motif chemokine is CX3CL1 (fractalkine).

11. The method of claim 1, wherein the one or more C-C motif chemokines are selected from the group consisting of CCL2 (MCP-1), CCL3 (MIP-1α), and CCL4 (MIP-1β).

12. The method of claim 1, wherein the one or more interleukin cytokines are selected from the group consisting of IL-1β, IL-18, and IL-6.

13. The method of claim 1, wherein the TNF superfamily cytokine is TNFα.

14. The method of claim 1, wherein the Fas inhibitor is selected from the group consisting of Met protein, derivatives, fragments, pharmaceutically acceptable salts thereof; Met protein derivative that does not bind HGF; Met-12, derivatives, fragments, pharmaceutically acceptable salts thereof; SEQ ID NOs: 1-8, derivatives, fragments, pharmaceutically acceptable salts thereof; or a gene therapy agents encoding the Fas inhibitor.

15. A method for preventing, treating or ameliorating an inflammation-mediated and/or complement-mediated disease or condition in a subject comprising administering to the subject a Fas inhibitor selected from the group consisting of Met protein, derivatives, fragments, pharmaceutically acceptable salts thereof; Met-12, derivatives, fragments, pharmaceutically acceptable salts thereof; SEQ ID NOs: 1-8, derivatives, fragments, pharmaceutically acceptable salts thereof; or a gene therapy agents encoding the Fas inhibitor, in an amount effective to inhibit Fas signaling, and thereby prevent, treat or ameliorate the inflammation-mediated and/or complement-mediated disease or condition in the subject.

16. The method of claim 15, wherein the subject has or is at risk of having the inflammation-mediated and/or complement-mediated disease or condition.

17. The method of claim 15, wherein the inflammation-mediated and/or complement-mediated disease or condition is selected from the group consisting of retinal disease, immunological disease, cancer, amyloid disease, an injury caused by ischemia or reperfusion, autoimmune disease, neurodegeneration, and diseases of the central nervous system.

18. The method of claim 15, wherein the Fas inhibitor is administered in a pharmaceutical composition comprising the Fas inhibitor and a pharmaceutically acceptable additive selected from the group consisting of carriers, excipients, disintegrators or disintegrating aids, binders, lubricants, coating agents, pigments, diluents, bases, dissolving agents or solubilizers, isotonic agents, pH regulators, stabilizers, propellants, and adhesives.

19. A method for preserving retinal ganglion cells and axon density, or preventing the loss of ganglion cells and axon density in a patient with glaucoma comprising administering to the subject a Fas inhibitor, a derivative thereof, a fragment thereof, a pharmaceutically acceptable salt thereof, or a gene therapy encoding the Fas inhibitor, wherein the preserving or preventing the loss of retinal ganglion cells and axon density, or preventing the loss thereof is due to at least one of the following:
inhibition of microglial/macrophage activation or recruitment;
inhibition of at least one of TNF-α, CCL2/MCP-1 or CCL3/MIP-1α gene or protein expression or concentration; or
reduction of IL-1β gene or protein expression or protein maturation, wherein the Fas inhibitor is administered to the subject in an amount effective to inhibit Fas signaling.

20. The method of claim 19, wherein the Fas inhibitor, a derivative thereof, a fragment thereof, a pharmaceutically acceptable salt thereof, or a gene therapy encoding the Fas inhibitor is administered in a pharmaceutical composition comprising the Fas inhibitor, a derivative thereof, a fragment thereof, a pharmaceutically acceptable salt thereof, or a gene therapy encoding the Fas inhibitor; and a pharmaceutically acceptable additive, wherein the additive is selected from the group consisting of carriers, excipients, disintegrators or disintegrating aids, binders, lubricants, coating agents, pigments, diluents, bases, dissolving agents or solubilizers, isotonic agents, pH regulators, stabilizers, propellants and adhesives.

21. The method of claim 19, wherein the composition is in a form selected from the group consisting of: solution, pill, ointment, suspension, eye drops, gel, cream, foam, spray, liniment, and powder.

22. The method of claim 19, wherein the administering is via an injection, wherein the injection is an intravitreal injection, intrathecal, intravenous or periocular injection.

23. The method of claim 19, wherein the composition further comprises at least one non-ionic surfactant selected from the group consisting of Polysorbate 80, Polysorbate 20, Poloxamer 407, and Tyloxapol.

24. A method of treating a subject having at least a 10% increase in the mRNA and/or protein expression level(s) of at least one of the following gene and/or protein in the subject's eye, as compared to a control:
at least one Fas-mediated inflammation-related gene or protein;
at least one Fas-mediated complement-related gene or protein;
Caspase 8;
one or more components of the inflammasome;
one or more C-X-C motif chemokines;
one or more C-X3-C motif chemokines;
one or more C-C motif chemokines;
toll-like receptor 4 (TLR4);
one or more interleukin cytokines;
one or more TNF superfamily cytokines; or
GFAP gene or protein expression or concentration,
the method comprising administering to the subject a Fas inhibitor.

25. A method of treating a subject having at least a 5% increase in the mRNA and/or protein expression level(s) of at least one of the following gene and/or protein in the subject's serum, plasma, whole blood, or cerebrospinal fluid, as compared to a control:
at least one Fas-mediated inflammation-related gene or protein;
at least one Fas-mediated complement-related gene or protein;
Caspase 8;
one or more components of the inflammasome;
one or more C-X-C motif chemokines;
one or more C-X3-C motif chemokines;
one or more C-C motif chemokines;
toll-like receptor 4 (TLR4);
one or more interleukin cytokines;
one or more TNF superfamily cytokines; or
GFAP gene or protein expression or concentration,
the method comprising administering to the subject a Fas inhibitor.

26. A composition comprising a compound selected from the group consisting of Compounds 2-8.


SEQUENCE LISTING (8 sequences)

SEQ ID NO: 1 - protein, 12 aa, Artificial Sequence
  Artificial C-terminal amide peptide of Met-12
  MOD_RES (12)..(12): AMIDATION
HHIYLGAVNYIY

SEQ ID NO: 2 - protein, 12 aa, Artificial Sequence
  Artificial derivative of Met-12
  MOD_RES (1)..(6): Desamino modification
  MOD_RES (8)..(12): Desamino modification
YIYNVAGLYIHH

SEQ ID NO: 3 - protein, 12 aa, Artificial Sequence
  Artificial derivative of C-terminal amide peptide of Met-12
  MOD_RES (1)..(12): Desamino modification
  MOD_RES (12)..(12): AMIDATION
YIYNVAGLYIHH

SEQ ID NO: 4 - protein, 12 aa, Artificial Sequence
  Artificial derivative of C-terminal amide peptide of Met-12
  MOD_RES (1)..(12): Desamino modification
  MOD_RES (12)..(12): AMIDATION
YIYNVAGLYIHH

SEQ ID NO: 5 - protein, 12 aa, Artificial Sequence
  Artificial derivative of C-terminal amide peptide of Met-12
  MOD_RES (1)..(12): Desamino modification
  MOD_RES (12)..(12): AMIDATION
YVYNVAGLYVHH

SEQ ID NO: 6 - protein, 12 aa, Artificial Sequence
  Artificial derivative of C-terminal amide peptide of Met-12
  MOD_RES (1)..(12): Desamino modification
  MOD_RES (12)..(12): AMIDATION
YVYNVAGLYVHH

SEQ ID NO: 7 - protein, 12 aa, Artificial Sequence
  Artificial derivative of Met-12
  MOD_RES (1)..(12): Hydroxy-desamino modification
  MOD_RES (2)..(2): aIle
  MOD_RES (10)..(10): aIle
YIYNVAGLYIHH

SEQ ID NO: 8 - protein, 12 aa, Artificial Sequence
  Artificial derivative of C-terminal amide peptide of Met-12
  MOD_RES (1)..(12): Desamino modification
  MOD_RES (12)..(12): AMIDATION (piperazine amide)
YVYNVAGLYVHH